(19) United States
(12) Patent Application Publication
(10) Pub. No.: US 2026/0306452 A1
(43) Pub. Date: Oct. 8, 2026

(54) 5-METHOXY-N,N-DIMETHYLTRYPTAMINE (5-MEO-DMT) PHARMACEUTICAL COMPOSITIONS

(71) Applicant: Beckley Psytech Limited, Oxford (GB)

(72) Inventors: Cosmo FEILDING-MELLEN, Oxford (GB); Timothy MASON, Oxford (GB)

(21) Appl. No.: 19/479,001

(22) PCT Filed: Apr. 29, 2024
(86) PCT No.: PCT/GB2024/051133

§ 371 Date: Oct. 27, 2025

(30) Foreign Application Priority Data
Apr. 27, 2023 (GB) 2306256.5

(51) Int. Cl. A61K 31/4045; A61K 9/00

(52) U.S. Cl. CPC A61K 31/4045; A61K 9/0043

(57) ABSTRACT

This invention relates to 5-methoxy-N,N-dimethyltryptamine (5-MeO-DMT) pharmaceutical compositions, more particularly intranasal pharmaceutical compositions of pharmaceutically acceptable salts of 5-MeO-DMT, and methods of administration and treatment using the same.

### FIELD OF THE INVENTION

This invention relates to 5-methoxy-N,N-dimethyltryptamine (5-MeO-DMT) pharmaceutical compositions, more particularly intranasal pharmaceutical compositions of pharmaceutically acceptable salts of 5-MeO-DMT, and methods of administration and treatment using the same.

### BACKGROUND OF THE INVENTION

5-methoxy-N,N-dimethyltryptamine is a pharmacologically active compound of the tryptamine class and has the chemical formula:

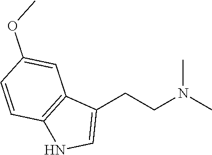 [SMILES: COc1ccc2[nH]cc(CCN(C)C)c2c1]

5-MeO-DMT is a psychoactive/psychedelic substance found in nature. Man-made salts of 5-MeO-DMT are also known in the art e.g. Sherwood, Alexander M., et al. “Synthesis and Characterization of 5-MeO-DMT succinate for clinical use.” ACS omega 5.49 (2020): 32067-32075 discloses the hydrochloride salt of 5-MeO-DMT. However, 5-MeO-DMT, and salts thereof, are not well understood and methods of administration of this compound, and the salts thereof, have not been well explored. For example, it has been found that some subjects being treated intranasally with a pharmaceutically effective amount of a salt of 5-MeO-DMT, may have rapid and intense psychedelic experiences and/or may experience some physical discomfort. For the sake of brevity, the term ‘5-MeO-DMT’ used herein, may also be understood to be referring to the salts of 5-MeO-DMT.

There remains a need in the art for improved pharmaceutical compositions comprising 5-MeO-DMT, and the salts thereof, and methods of administration and treatment using the same.

### DESCRIPTION OF THE INVENTION

In a first aspect of the invention, there is provided an intranasal pharmaceutical composition comprising a salt of 5-methoxy-N,N-dimethyltryptamine (5-MeO-DMT salt) for use, as a medicament, wherein the pharmaceutical composition comprises: a first dose of the 5-MeO-DMT salt, for administration to a first nostril of a subject; and a second dose of the 5-MeO-DMT salt, for second administration to a second nostril of the subject.

It has been found that a single intranasal dose of 5-MeO-DMT benzoate can rapidly induce intense psychedelic experiences, with subjects reaching scores of >3 in the 30-item Mystical Experience Questionnaire (MEQ30). The MEQ30 is a self-report measure that has been used to measure mystical-type experiences in studies of psychedelics. It is known that the occurrence of a mystical experience, following treatment with a psychedelic, is a primary predictor of therapeutic outcomes in patients. Typically, a complete mystical experience is defined as scoring 60% or more on all four factors of the MEQ30. The four factors of the MEQ30 are: mystical, positive mood, transcendence of time and space, and ineffability. Subjects being treated with the 5-MeO-DMT salt are coached prior to dosing, but nevertheless for some subjects, a rapid onset of an intense Mystical Experience can be unsettling. In such a scenario, the medical practitioners and associated support system is also put under an additional monitoring and care load. So, the frequency/likelihood of rapid onset of an intense Mystical Experience using the 5-MeO-DMT salt should be minimised or substantially eliminated.

While the overall tolerability profile of intranasal 5-MeO-DMT benzoate is good (and better than the corresponding and better known chloride salt), some subjects do experience some physical discomfort. For example, some subjects report nasal discomfort when treated with a single intranasal dose of a salt of 5-MeO-DMT. Additionally, some subjects experienced nausea and vomiting when given a higher single dose of a salt of 5-MeO-DMT. So, the frequency/likelihood of physical discomfort when using the 5-MeO-DMT salt should be minimised or substantially eliminated.

Herein disclosed, the invention surprisingly ameliorates/overcomes the limitations mentioned herein above, i.e. when compared to the use of a single intranasal dose of a pharmaceutically effective amount of a salt of 5-MeO-DMT.

Without wishing to be bound by theory, it may be understood that by administering the pharmaceutical composition of the invention to a subject (where the first dose of 5-MeO-DMT is firstly administered to a first nostril, and the second dose of the 5-MeO-DMT is secondly administered to the second nostril of the subject), a biphasic administration of the 5-MeO-DMT is received by the subject. With reference to FIG. 7, this can be seen in the m-like biphasic curve obtained in the measured mean plasma concentration levels, where there is a first initial peak followed by a second peak. Splitting the dose in this manner provides several benefits. Firstly, rapid onset of an intense mystical experience is ameliorated/overcome. Without wishing to be bound by theory, it is believed that the biphasic profile provided by the pharmaceutical composition of the invention softens/smooths out the overall Mystical Experience, without sacrificing the overall benefit. Moreover, the invention provides the benefit that a subject can abort the treatment after the first dose (i.e. not taking the second dose) if they feel that the Mystical Experience is too intense or the onset is too rapid (this is not possible with a single larger dose). These benefits reduce the care load placed on any medical practitioners/carers. The subjects also experience less physical discomfort. Without wishing to be bound by theory, it is believed that the first dose in effect conditions the subject to better tolerate the second dose (which may be larger), so for example any uncomfortable sensation in the nostrils is not noticed, or is less noticeable. Without wishing to be bound by theory, it is believed that having a Mystical Experience, partial Mystical Experience, or the onset of a Mystical Experience lessens the noticeability of any uncomfortable physical sensations in the subject. As such, the biphasic approach used in the invention synergistically provides a useful safety break, lessens the care load on medical support staff in the case of an unwelcome rapid/intense Mystical Experience, and is better overall physically tolerated by the subject.

In an embodiment, the 5-MeO-DMT salt is amorphous, crystalline or is substantially polymorphically pure. In an embodiment, the pharmaceutical composition comprises one or more pharmaceutically acceptable carriers or excipients. In an embodiment, the pharmaceutical composition is a dry powder. In an embodiment, the 5-MeO-DMT salt is non-hygroscopic. Advantageously, non-hygroscopic products are easier to formulate and handle and are less prone to degradation. In an embodiment, the pharmaceutical composition comprises a mucoadhesive, optionally the mucoadhesive is hydroxypropyl methyl cellulose (HPMC). In an embodiment, the pharmaceutical composition does not comprise hydroxypropyl methyl cellulose (HPMC).

In an embodiment, the mass ratio of the first dose of 5-MeO-DMT salt to the second dose of 5-MeO-DMT salt is from 1:11 to 11:1, optionally from 2:10 to 10:2, further optionally from 4:8 to 8:4, and still further optionally the mass ratio is the same. Advantageously, splitting the dose in this way allows the benefit of the invention to be tuned by the medical practitioner to suit need and the particular circumstances of the patient being treated.

In an embodiment, the first dose of the 5-MeO-DMT salt is lower, equal, or higher than the second dose of the 5-MeO-DMT salt. In an embodiment, the first dose of the 5-MeO-DMT salt is lower than the second dose of the 5-MeO-DMT salt. It may be envisioned that a smaller dose is followed by a larger dose, but this could be reversed or the doses could be equal. Additionally, the combined dose could be split into more than 2 parts e.g. 3 or 4 parts. It is also possible that an additional active substance could be included in the first or second dose, or could be present in both doses, or is in one or more doses that follow, intervene or proceed the first or second doses. Again, this could be tuned by the medical practitioner to suit need and the particular circumstance of the patient being treated.

In an embodiment, the anion of the first dose of the 5-MeO-DMT salt is a benzoate, hydrobromide, hydrochloride, phosphate, fumarate, oxalate, tartrate, benzenesulfonate, tosylate, glycolate, ketoglutarate, malate, saccharinate or succinate salt. In an embodiment, the anion of the second dose of the 5-MeO-DMT salt is a benzoate, hydrobromide, hydrochloride, phosphate, fumarate, oxalate, tartrate, benzenesulfonate, tosylate, glycolate, ketoglutarate, malate, saccharinate or succinate salt. The salt type can be selected by the medical practitioner or formulator to suit need and the particular circumstance of the patient being treated or the physical requirements of the pharmaceutical composition needed.

In an embodiment, one or both of the first dose of the 5-MeO-DMT and the second dose of the 5-MeO-DMT are the free base. In an embodiment, both the first and second dose cannot both be the freebase. In some circumstances use of the free base could be beneficial. In an embodiment, the first dose of the 5-MeO-DMT salt and the second dose of the 5-MeO-DMT salt are the same salt. In an embodiment, the first dose of the 5-MeO-DMT salt and the second dose of the 5-MeO-DMT salt are not the same salt. Again, the salt type can be selected by the medical practitioner or formulator to suit need and the particular circumstance of the patient being treated or the physical requirements of the pharmaceutical composition needed.

In an embodiment, the 5-MeO-DMT salt is 5-MeO-DMT benzoate. Advantageously, the benzoate salt has shown good irritation tolerability, in particular when compared to the better known chloride salt, and has a good stability profile. In an embodiment, the 5-MeO-DMT benzoate is crystalline. In an embodiment, the crystalline 5-MeO-DMT benzoate is characterised by one or more peaks in an X-ray powder diffraction (XRPD) diffractogram at a 2θ value of 17.5°±0.1°, 17.7°±0.1° and 21.0°±0.1° using an X-ray wavelength of 1.5406 Å.

In an embodiment, the first and second dose of the 5-MeO-DMT salt benzoate forms a combined dose of 30, 20, 18, 16, 15, 14, 13, 12, 11, 10, 9, 8, 7, 6, 5, 4, 3, 2 or 1 mg of the 5-MeO-DMT benzoate. In an embodiment, the first and second dose of the 5-MeO-DMT salt benzoate forms a combined dose of 12 mg of the 5-MeO-DMT benzoate. In an embodiment, the first dose of 5-MeO-DMT benzoate is 4 mg and the second dose of 5-MeO-DMT benzoate is 8 mg. The 4 mg first dose followed by the second 8 mg dose of 5-MeO-DMT benzoate has shown good efficacy.

In an embodiment, the pharmaceutical composition comprises 5-MeO-DMT hydrobromide in place of the 5-MeO-DMT benzoate, and wherein the pharmaceutical composition comprises substantially the same dosage amount of the active 5-MeO-DMT cation. Advantageously, the hydrobromide salt is substantially non-hygroscopic.

It should be appreciated that different salts of 5-MeO-DMT will have different formula weights. For example the hydrochloride, hydrobromide and benzoate have, respectively, formula weights of about 254.8 g/mol, 299.2 g/mol, 340.4 g/mol and the free base of 5-MeO-DMT 218.3 g/mol. So this is the amount of substance that is required to give 1 mol of the active agent. So, for example for the salt, the dosage amount may be the equivalent amount of the free base delivered when the salt is taken. So 100 mg dosage amount of 5-MeO-DMT corresponds to 117 mg of the hydrochloride salt (i.e. both providing the same molar amount of the active substance). The greater mass of the salt needed is due to the larger formula weight of the hydrogen chloride salt (i.e. 218.3 g/mol for the free base as compared to 254.8 g/mol for the salt). In an embodiment, Xmg of a 5-MeO-DMT salt refers to the dosage amount of said salt which equates to Xmg of the 5-MeO-DMT freebase. Therefore, a pharmaceutical composition comprising 12 mg 5-MeO-DMT benzoate refers to a pharmaceutical composition comprising 18.7 mg 5-MeO-DMT benzoate which equates to 12 mg of 5-MeO-DMT freebase. Similarly, for a deuterated or triturated version of 5-MeO-DMT (also considered within the scope of the invention), a slight increase in mass can be expected due to the increased formula weight of these isotopic compounds. Unless stated otherwise, the mass (mg) of 5-MeO-DMT refers to the mass of benzoate salt (and so the equivalent molar amount of the 5-MeO-DMT active agent). Accordingly, with reference to the other salts mentioned herein, the appropriate mass of the other salt can be scaled accordingly using ratios of the formula weights. These masses of salts are normally rounded up or down to suit need. This rounding may be to the nearest whole, half, quarter or tenth of a milligram (mg). Splitting of a combined dose will typically be done to whole numbers so 3.5 and 6.5 mg (combined total of 10 mg) may be formulated to 3 and 7 mg respectively.

In an embodiment, the 5-MeO-DMT hydrobromide is crystalline. In an embodiment, the crystalline 5-MeO-DMT hydrobromide is characterised by one or more of: peaks in an x-ray powder diffraction (XRPD) diffractogram at 2θ values of 14.6°±0.1°, 16.8°±0.1°, 20.8°±0.1°, 24.3°±0.1°, 24.9°±0.1°, and 27.5°±0.1° as measured using an x-ray wavelength of 1.5406 Å; peaks in an x-ray powder diffraction (XRPD) diffractogram at 2θ values of 14.6°±0.1°, 21.6°±0.1°, and 24.3°±0.1° as measured using an x-ray wavelength of 1.5406 Å; peaks in an x-ray powder diffraction (XRPD) diffractogram at 2θ values of 18.6°±0.1°, 19.7°±0.1°, and 24.8°±0.1° as measured using an x-ray wavelength of 1.5406; or peaks in an x-ray powder diffraction (XRPD) diffractogram at 2θ values of 14.6°±0.1°, 20.8°±0.1°, 21.6°±0.1°, 24.3°±0.1°, and 25.4°±0.1° as measured using an x-ray wavelength of 1.5406 Å.

In an embodiment, the second dose of the 5-MeO-DMT salt is for administration within 30, 20, 15, 10, 5 or 2 minutes after the first dose of the 5-MeO-DMT salt. In an embodiment, the second dose of the 5-MeO-DMT salt is for administration while the subject being treated is experiencing one or more mystical effects from the first dose of the 5-MeO-DMT salt.

In an embodiment, the second dose of the 5-MeO-DMT salt is for administration prior to the expiry of the mystical effects from the first dose of the 5-MeO-DMT salt. In an embodiment, the first dose of the 5-MeO-DMT salt provides a first peak and the second dose of the 5-MeO-DMT salt provides a second peak, and wherein the peaks overlap, but wherein the maximum of each peak is distinct; optionally the height of the first peak maximum is lower than the height of the second peak maximum. Advantageously, it is expected that the second dose will be given within a relatively short window after the first dose, in particular while the first dose is taking (or having an) effect on the subject.

In an embodiment, the first dose of the 5-MeO-DMT salt provides a first peak and the second dose of the 5-MeO-DMT salt provides a second peak, and where the peaks overlap, together forming a combined ‘m’ shaped curve, optionally the combined ‘m’ shaped curve is lopsided, further optionally wherein the first peak is lower than the second peak. In an embodiment, the first dose of the 5-MeO-DMT salt provides a first peak and the second dose of the 5-MeO-DMT salt a second peak as substantially shown in FIG. 7.

In an embodiment, the first dose of the 5-MeO-DMT salt and second dose of the 5-MeO-DMT salt are together better tolerated by a subject than a single administered dose which is the same or larger.

In an embodiment, there is provided a pharmaceutical kit comprising a first and second dose of the invention or any embodiment of the invention. In an aspect, there is provided a pharmaceutical kit comprising a first single use intranasal applicator comprising a dry powder intranasal pharmaceutical composition of 5-MeO-DMT benzoate and a second single use intranasal applicator comprising a dry powder intranasal pharmaceutical composition of 5-MeO-DMT benzoate, wherein the dose of 5-MeO-DMT benzoate in the second intranasal applicator is higher than that in the first, optionally, the dose of 5-MeO-DMT benzoate in the first applicator is 4 mg and the dose in the second applicator is 8 mg.

Herein, the terms ‘pharmaceutical composition’ and ‘pharmaceutical composition’ are used interchangeably. In an embodiment, there is provided a pharmaceutical composition comprising 5-methoxy-N,N-dimethyltryptamine (5-MeO-DMT) for use in a method of treating depression, wherein the pharmaceutical composition is administered intranasally as follows: a first dose of the pharmaceutical composition is administered into a nostril; and a second dose of the pharmaceutical composition is administered into the other nostril.

In an embodiment, the pharmaceutical composition comprises one or more pharmaceutically acceptable carriers or excipients. In an embodiment, the second dose of the pharmaceutical composition is higher than the first dose. In an embodiment, the second dose of the pharmaceutical composition is administered not more than 30 minutes after the first dose. In an embodiment, the second dose of the pharmaceutical composition is administered not more than 20 minutes after the first dose.

In an embodiment, the second dose of the pharmaceutical composition is administered not more than 15 minutes after the first dose. In an embodiment, the second dose of the pharmaceutical composition is administered not more than 10 minutes after the first dose. In an embodiment, the pharmaceutical composition comprises the benzoate salt of 5-MeO-DMT. In an embodiment, the pharmaceutical composition comprises crystalline 5-MeO-DMT benzoate. In an embodiment, the crystalline 5-MeO-DMT benzoate is characterised by a peak in an X-ray powder diffraction (XRPD) diffractogram at a 2θ value of 17.5°±0.10 using an X-ray wavelength of 1.5406 Å. In an embodiment, the crystalline 5-MeO-DMT benzoate is characterised by a peaks in an X-ray powder diffraction (XRPD) diffractogram at 2θ values of 17.5°±0.1° and 17.7°±0.1° using an X-ray wavelength of 1.5406 Å. In an embodiment, the crystalline 5-MeO-DMT benzoate is characterised by a peaks in an X-ray powder diffraction (XRPD) diffractogram at 2θ values of 17.5°±0.1°, 17.7°±0.1° and 21.0°±0.1° using an X-ray wavelength of 1.5406 Å.

In an embodiment, the pharmaceutical composition is formulated as a dry powder. In an embodiment, the pharmaceutical composition comprises hydroxypropyl methyl cellulose (HPMC). In an embodiment, the pharmaceutical composition is formulated as a dry powder. In an embodiment, the pharmaceutical composition comprises the benzoate salt of 5-MeO-DMT and wherein the first dose of 5-MeO-DMT is 4 mg and the second dose is 8 mg. In an embodiment, the second dose of 5-MeO-DMT is administered 5 minutes, 10 minutes or 15 minutes after the first dose. In an embodiment, the pharmaceutical composition comprises the hydrobromide salt of 5-MeO-DMT. In an embodiment, the pharmaceutical composition comprises the crystalline hydrobromide salt of 5-MeO-DMT. In an embodiment, the pharmaceutical composition comprises the crystalline hydrobromide salt of 5-MeO-DMT as characterised by one or more of: peaks in an x-ray powder diffraction (XRPD) diffractogram at 2θ values of 14.6°±0.1°, 16.8°±0.1°, 20.8°±0.1°, 24.3°±0.1°, 24.9°±0.1°, and 27.5°±0.1° as measured using an x-ray wavelength of 1.5406 Å; peaks in an x-ray powder diffraction (XRPD) diffractogram at 2θ values of 14.6°±0.1°, 21.6°±0.1°, and 24.3°±0.1° as measured using an x-ray wavelength of 1.5406 Å; peaks in an x-ray powder diffraction (XRPD) diffractogram at 2θ values of 18.6°±0.1°, 19.7°±0.1°, and 24.8°±0.1° as measured using an x-ray wavelength of 1.5406; or peaks in an x-ray powder diffraction (XRPD) diffractogram at 2θ values of 14.6°±0.1°, 20.8°±0.1°, 21.6°±0.1°, 24.3°±0.1°, and 25.4°±0.1° as measured using an x-ray wavelength of 1.5406 Å.

In an embodiment, the first dose of 5-MeO-DMT is 4 mg and the second dose is 8 mg. In an embodiment, the second dose of 5-MeO-DMT is administered 5 minutes, 10 minutes or 15 minutes after the first dose. In an embodiment, the pharmaceutical composition comprises the hydrochloride, phosphate, fumarate, oxalate, tartrate, benzenesulfonate, tosylate, glycolate, ketoglutarate, malate, saccharinate or succinate salt of 5-MeO-DMT. In an embodiment, the pharmaceutical composition comprises a crystalline form of the hydrochloride, phosphate, fumarate, oxalate, tartrate, benzenesulfonate, tosylate, glycolate, ketoglutarate, malate, saccharinate or succinate salt of 5-MeO-DMT. In an embodiment, 5-MeO-DMT is provided for use as a medicament, wherein 5-MeO-DMT is administered in a biphasic fashion. In an embodiment, 5-MeO-DMT is provided for use as a medicament, wherein 5-MeO-DMT is administered in two doses. In an embodiment, there is provided 5-MeO-DMT for use as a medicament, wherein 5-MeO-DMT is administered as follows: a first dose of the 5-MeO-DMT is administered to a subject; and a second dose of the 5-MeO-DMT is administered to the subject.

In an embodiment, the second dose is administered not more than 60 minutes, not more than 50 minutes, not more than 40 minutes, not more than 30 minutes, not more than 25 minutes, not more than 20 minutes, not more than 15 minutes, not more than 10 minutes or not more than 5 minutes after the first dose. In an embodiment, the 5-MeO-DMT is administered via an enteral route, a parenteral route, a transnasal route, via a route that involves the respiratory tract epithelium, via a vaginal route, via transdermal route or via an intraosseous route. In an embodiment, the 5-MeO-DMT is administered intranasally.

In an embodiment, the first dose of 5-MeO-DMT and the second dose of 5-MeO-DMT are administered via the same route. In an embodiment, the first dose of 5-MeO-DMT and the second dose of 5-MeO-DMT are administered via different routes. In an embodiment, the 5-MeO-DMT is administered as the free base. In an embodiment, the 5-MeO-DMT is administered as a salt. In an embodiment, the 5-MeO-DMT is administered as a crystalline salt. In an embodiment, the 5-MeO-DMT is administered as a polymorphic salt form. In an embodiment, the 5-MeO-DMT is administered as a polymorph of a 5-MeO-DMT salt. In an embodiment, the 5-MeO-DMT is administered as the benzoate, fumarate, citrate, acetate, succinate, halide, fluoride, chloride, bromide, iodide, oxalate, or triflate salt. In an embodiment, the 5-MeO-DMT is administered as the benzoate salt. In an embodiment, the 5-MeO-DMT is administered as the hydrochloride salt. In an embodiment, the 5-MeO-DMT is administered as the hydrobromide salt. In an embodiment, the 5-MeO-DMT salt is administered in an amorphous form. In an embodiment, the 5-MeO-DMT salt is administered in a crystalline form.

In an embodiment, the 5-MeO-DMT is administered as a crystalline form of the benzoate salt. Crystalline forms of the benzoate salt are disclosed in WO2021250434 and are incorporated herein. Crystalline forms of the hydrochloride salt are also disclosed in WO2021250434 and are incorporated herein. In an embodiment, crystalline 5-MeO-DMT hydrochloride is characterised by peaks in an X-ray powder diffraction (XRPD) diffractogram at 2θ values of 9.2°±0.1°, 12.2°±0.1°, 14.1°±0.1°, 15.0°±0.1°, 18.5°±0.1°, and 19.5°±0.1°, as measured using an X-ray wavelength of 1.5406 Å. In an embodiment, the 5-MeO-DMT salt is a non-hygroscopic salt. The inventors have surprisingly discovered that 5-MeO-DMT hydrobromide is a non-hygroscopic salt of 5-MeO-DMT. The tartrate salt of 5-MeO-DMT is moderately hygroscopic, the tosylate salt and the phosphate salt are both slightly hygroscopic. The inventors have further surprisingly discovered that 5-MeO-DMT hydrobromide, whilst being non-hygroscopic, has high solubility compared to other moderately hygroscopic salts of 5-MeO-DMT for example the benzoate or oxalate salts. The non-hygroscopic, highly soluble HBr salt of 5-MeO-DMT therefore affords the advantage of removing the need for costly and burdensome processing measures, for example the need for low humidity manufacturing environment. The high solubility of the HBr salt of 5-MeO-DMT also facilitates the use of simplified solid pharmaceutical compositions without the need for costly solubility enhancement techniques. The inventors have further surprisingly discovered multiple polymorphic forms of crystalline 5-MeO-DMT hydrobromide, including a form referred to as form/pattern 2 with desirable qualities.

Hygroscopicity is the phenomenon of attracting and holding water molecules via either adsorption or absorption from the surrounding environment. Pharmaceuticals that pick up less than 0.2% moisture at 80% RH are considered non hygroscopic. Pharmaceuticals that pick up between 0.2% and 2.0% moisture at 80% RH are considered slightly hygroscopic. Pharmaceuticals that pick up between 2.0% and 15.0% moisture at 80% RH are considered moderately hygroscopic. Pharmaceuticals that pick up more than 15.0% moisture at 80% RH are considered very hygroscopic. Hygroscopic substances are difficult to handle and costly and burdensome measures must be taken in order to ensure they are not exposed to moisture during process and pharmaceutical composition. Exposed to moisture, hygroscopic substances can take on water and convert to a hydrous form. This presents several disadvantages. First, the hydrous forms may have the disadvantage of being less bioavailable and less dissoluble than the anhydrous forms. Second, the variation in the amount of hydrous versus anhydrous substance from batch to batch could fail to meet specifications set by drug regulatory agencies. Third, processes like milling may cause the drug substance to adhere to manufacturing equipment which may further result in processing delay, increased operator involvement, increased cost, increased maintenance and lower production yield. Fourth, in addition to problems caused by introduction of moisture during the processing of these hygroscopic substances, the potential for absorbance of moisture during storage and handling would adversely affect the dissolubility of the drug substance. Thus shelf-life of the product could be significantly decreased and/or packaging costs could be significantly increased.

In an embodiment, the crystalline 5-MeO-DMT hydrobromide is characterised by one or more of: peaks in an x-ray powder diffraction (XRPD) diffractogram at 2θ values of 14.6°±0.1°, 16.8°±0.1°, 20.8°±0.1°, 24.3°±0.1°, 24.9°±0.1°, and 27.5°±0.1° as measured using an x-ray wavelength of 1.5406 Å; peaks in an x-ray powder diffraction (XRPD) diffractogram at 2θ values of 14.6°±0.1°, 21.6°±0.1°, and 24.3°±0.1° as measured using an x-ray wavelength of 1.5406 Å; peaks in an x-ray powder diffraction (XRPD) diffractogram at 2θ values of 18.6°±0.1°, 19.7°±0.1°, and 24.8°±0.1° as measured using an x-ray wavelength of 1.5406; or peaks in an x-ray powder diffraction (XRPD) diffractogram at 2θ values of 14.6°±0.1°, 20.8°±0.1°, 21.6°±0.1°, 24.3°±0.1°, and 25.4°±0.1° as measured using an x-ray wavelength of 1.5406 Å. In an embodiment, the crystalline 5-MeO-DMT hydrobromide is characterised by one or more peaks in an XRPD diffractogram as detailed in the Tables below:

### XRPD Peak Data for Hydrobromide Pattern 1.

| Peak No. | Angle 2 θ | d Value | Rel. Intensity |
|---|---|---|---|
| 1 | 6.202° | 14.238 | 0.187 |
| 2 | 9.099° | 9.711 | 0.022 |
| 3 | 12.115° | 7.299 | 0.004 |
| 4 | 12.359° | 7.156 | 0.192 |
| 5 | 13.322° | 6.641 | 0.002 |
| 6 | 14.046° | 6.300 | 0.229 |
| 7 | 14.667° | 6.035 | 0.104 |
| 8 | 15.219° | 5.817 | 0.007 |
| 9 | 16.722° | 5.297 | 0.002 |
| 10 | 18.566° | 4.775 | 1.000 |
| 11 | 18.780° | 4.721 | 0.030 |
| 12 | 19.740° | 4.494 | 0.361 |
| 13 | 20.937° | 4.239 | 0.026 |
| 14 | 22.900° | 3.880 | 0.026 |
| 15 | 23.540° | 3.776 | 0.106 |
| 16 | 24.169° | 3.679 | 0.016 |
| 17 | 24.824° | 3.584 | 0.299 |
| 18 | 25.714° | 3.462 | 0.005 |
| 19 | 26.109° | 3.410 | 0.014 |
| 20 | 26.746° | 3.331 | 0.007 |
| 21 | 27.427° | 3.249 | 0.042 |
| 22 | 28.245° | 3.157 | 0.036 |
| 23 | 28.771° | 3.101 | 0.002 |
| 24 | 30.020° | 2.974 | 0.004 |
| 25 | 30.556° | 2.923 | 0.006 |
| 26 | 31.132° | 2.871 | 0.020 |

### XRPD Peak Data for Hydrobromide Pattern 1. (2 d.p.)

| Peak No. | Angle 2 θ | d Value | Rel. Intensity |
|---|---|---|---|
| 1 | 6.20° | 14.24 | 0.19 |
| 2 | 9.10° | 9.71 | 0.02 |
| 3 | 12.12° | 7.30 | 0.00 |
| 4 | 12.36° | 7.16 | 0.19 |
| 5 | 13.32° | 6.64 | 0.00 |
| 6 | 14.05° | 6.30 | 0.23 |
| 7 | 14.67° | 6.04 | 0.10 |
| 8 | 15.22° | 5.82 | 0.01 |
| 9 | 16.72° | 5.30 | 0.00 |
| 10 | 18.57° | 4.78 | 1.00 |
| 11 | 18.78° | 4.72 | 0.03 |
| 12 | 19.74° | 4.49 | 0.36 |
| 13 | 20.94° | 4.24 | 0.03 |
| 14 | 22.90° | 3.88 | 0.03 |
| 15 | 23.54° | 3.78 | 0.11 |
| 16 | 24.17° | 3.68 | 0.02 |
| 17 | 24.82° | 3.58 | 0.30 |
| 18 | 25.71° | 3.46 | 0.01 |
| 19 | 26.11° | 3.41 | 0.01 |
| 20 | 26.75° | 3.33 | 0.01 |
| 21 | 27.43° | 3.25 | 0.04 |
| 22 | 28.25° | 3.16 | 0.04 |
| 23 | 28.77° | 3.10 | 0.00 |
| 24 | 30.02° | 2.97 | 0.00 |
| 25 | 30.56° | 2.92 | 0.01 |
| 26 | 31.13° | 2.87 | 0.02 |

### XRPD Peak Data for Hydrobromide Pattern 1 (1 d.p.).

| Peak No. | Angle 2 θ | d Value | Rel. Intensity |
|---|---|---|---|
| 1 | 6.2° | 14.2 | 0.2 |
| 2 | 9.1° | 9.7 | 0.0 |
| 3 | 12.1° | 7.3 | 0.0 |
| 4 | 12.4° | 7.2 | 0.2 |
| 5 | 13.3° | 6.6 | 0.0 |
| 6 | 14.0° | 6.3 | 0.2 |
| 7 | 14.7° | 6.0 | 0.1 |
| 8 | 15.2° | 5.8 | 0.0 |
| 9 | 16.7° | 5.3 | 0.0 |
| 10 | 18.6° | 4.8 | 1.0 |
| 11 | 18.8° | 4.7 | 0.0 |
| 12 | 19.7° | 4.5 | 0.4 |
| 13 | 20.9° | 4.2 | 0.0 |
| 14 | 22.9° | 3.9 | 0.0 |
| 15 | 23.5° | 3.8 | 0.1 |
| 16 | 24.2° | 3.7 | 0.0 |
| 17 | 24.8° | 3.6 | 0.3 |
| 18 | 25.7° | 3.5 | 0.0 |
| 19 | 26.1° | 3.4 | 0.0 |
| 20 | 26.7° | 3.3 | 0.0 |
| 21 | 27.4° | 3.2 | 0.0 |
| 22 | 28.2° | 3.2 | 0.0 |
| 23 | 28.8° | 3.1 | 0.0 |
| 24 | 30.0° | 3.0 | 0.0 |
| 25 | 30.6° | 2.9 | 0.0 |
| 26 | 31.1° | 2.9 | 0.0 |

In an embodiment, the crystalline 5-MeO-DMT hydrobromide is characterised by one or more of One or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, twenty three or more, twenty four or more, twenty five or more, or twenty six peaks in an XRPD diffractogram as detailed in the Tables above; One or more, two or more, three or more, four or more or five or more peaks in an XRPD diffractogram with a relative intensity of over 0.1, 0.2, 0.3, 0.4, 0.5, 0.6, 0.7, 0.8 or 0.9 as detailed in the Tables above; A weight loss of about 0.45% between 25-220° C., as measured by TGA thermogram; A weight loss of about 0.3560.55 between 25-220° C., as measured by TGA thermogram; A weight loss of about 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54 or 0.55° between 25-220° C., as measured by TGA thermogram; A weight loss of about 0.1-1.0% between 25-220° C., as measured by TGA thermogram; A weight loss of about 0.1, 0.2, 0.3, 0.4, 0.5, 0.6, 0.7, 0.8, 0.9 or 1.0% between 25-220° C., as measured by TGA thermogram; A melting endothermic event with an onset of around 148.7° C. and an enthalpy of 97.3 J/g, as measured in a DSC thermogram; A melting endothermic event with an onset of around 143-153° C. and an enthalpy of around 92-102 J/g, as measured in a DSC thermogram; A melting endothermic event with an onset of around 143, 144, 145, 146, 147, 148, 149, 150, 151, 152 or 153 and an enthalpy of around 92, 93, 94, 95, 96, 97, 98, 99, 100, 101, or 102 J/g, as measured in a DSC thermogram; A vitrification around 34.8° C., as measured in a DSC thermogram with a cooling ramp of 10° C./min from 220° C. to −90° C.; A vitrification around 30-40° C. as measured in a DSC thermogram with a cooling ramp of 10° C./min from 220° C. to −90° C.; A vitrification around 30, 31, 32, 33, 34, 35, 36, 37, 38, 39, or 40° C. as measured in a DSC thermogram with a cooling ramp of 10° C./min from 220° C. to −90° C.; A glass transition around 42.2° C., as measured in a DSC thermogram with a cooling ramp of 10° C./min from 220° C. to −90° C.; A glass transition around 37-47° C., as measured in a DSC thermogram with a cooling ramp of 10° C./min from 220° C. to −90° C.; and/or A glass transition around 37, 38, 39, 40, 41, 42, 43, 44, 45, 46 or 47° C., as measured in a DSC thermogram with a cooling ramp of 10° C./min from 220° C. to −90° C.

In an embodiment, the crystalline 5-MeO-DMT hydrobromide is characterised by one or more peaks in an XRPD diffractogram as detailed in the Tables below:

### XRPD Peak Data for Hydrobromide Pattern 2.

| | | | Rel. |
|---|---|---|---|
| Peak No. | Angle 2 θ | d Value | Intensity |
| 1 | 12.693° | 6.968 | 0.053 |
| 2 | 14.633° | 6.049 | 0.384 |
| 3 | 16.792° | 5.275 | 0.075 |
| 4 | 17.175° | 5.159 | 0.160 |
| 5 | 20.750° | 4.277 | 0.209 |
| 6 | 21.561° | 4.118 | 1.000 |
| 7 | 22.574° | 3.936 | 0.087 |
| 8 | 22.740° | 3.907 | 0.111 |
| 9 | 24.334° | 3.655 | 0.722 |
| 10 | 24.871° | 3.577 | 0.195 |
| 11 | 25.441° | 3.498 | 0.366 |
| 12 | 26.421° | 3.371 | 0.106 |
| 13 | 27.484° | 3.243 | 0.174 |
| 14 | 28.558° | 3.123 | 0.074 |
| 15 | 29.072° | 3.069 | 0.066 |
| 16 | 29.388° | 3.037 | 0.126 |
| 17 | 31.314° | 2.854 | 0.057 |
| 18 | 31.652° | 2.825 | 0.071 |

### XRPD Peak Data for Hydrobromide Pattern 2. (2 d.p.)

| | | | Rel. |
|---|---|---|---|
| Peak No. | Angle 2 θ | d Value | Intensity |
| 1 | 12.69° | 6.97 | 0.05 |
| 2 | 14.63° | 6.05 | 0.38 |
| 3 | 16.79° | 5.28 | 0.08 |
| 4 | 17.18° | 5.16 | 0.16 |
| 5 | 20.75° | 4.28 | 0.21 |
| 6 | 21.56° | 4.12 | 1.00 |
| 7 | 22.57° | 3.94 | 0.09 |
| 8 | 22.74° | 3.91 | 0.11 |
| 9 | 24.33° | 3.66 | 0.72 |
| 10 | 24.87° | 3.58 | 0.20 |
| 11 | 25.44° | 3.50 | 0.37 |
| 12 | 26.42° | 3.37 | 0.11 |
| 13 | 27.48° | 3.24 | 0.17 |
| 14 | 28.56° | 3.12 | 0.07 |
| 15 | 29.07° | 3.07 | 0.07 |
| 16 | 29.39° | 3.04 | 0.13 |
| 17 | 31.31° | 2.85 | 0.06 |
| 18 | 31.65° | 2.83 | 0.07 |

### XRPD Peak Data for Hydrobromide Pattern 2. (2 d.p.)

| | | | Rel. |
|---|---|---|---|
| Peak No. | Angle 2 θ | d Value | Intensity |
| 1 | 12.7° | 7.0 | 0.1 |
| 2 | 14.6° | 6.1 | 0.4 |
| 3 | 16.8° | 5.3 | 0.1 |
| 4 | 17.2° | 5.2 | 0.2 |
| 5 | 20.8° | 4.3 | 0.2 |
| 6 | 21.6° | 4.1 | 1.0 |
| 7 | 22.6° | 3.9 | 0.1 |
| 8 | 22.7° | 3.9 | 0.1 |
| 9 | 24.3° | 3.7 | 0.7 |
| 11 | 24.9° | 3.6 | 0.2 |
| 12 | 25.4° | 3.5 | 0.4 |
| 13 | 26.4° | 3.4 | 0.1 |
| 14 | 27.5° | 3.2 | 0.2 |
| 15 | 28.6° | 3.1 | 0.1 |
| 16 | 29.1° | 3.1 | 0.1 |
| 17 | 29.4° | 3.0 | 0.1 |
| 18 | 31.3° | 2.9 | 0.1 |
| 19 | 31.7° | 2.8 | 0.1 |

In an embodiment, the crystalline 5-MeO-DMT hydrobromide is characterised by one or more of: One or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more peaks in an XRPD diffractogram as detailed in the Tables above; One or more, two or more, three or more, four or more or five or more peaks in an XRPD diffractogram with a relative intensity of over 0.1, 0.2, 0.3, 0.4, 0.5, 0.6, 0.7, 0.8 or 0.9 as detailed in the Tables above; A melting endothermic event with an onset of around 166.10° C. and an enthalpy of 104.04 J/g, as measured in a DSC thermogram; A melting endothermic event with an onset of around 160-170° C. and an enthalpy of 100-110 J/g, as measured in a DSC thermogram; A melting endothermic event with an onset of around 160, 161, 162, 163, 164, 165, 166, 167, 168, 169 or 170° C. and an enthalpy of 100, 101, 102, 103, 104, 105, 106, 107, 108, 109 or 110 J/g, as measured in a DSC thermogram; A vitrification around 42.7° C., as measured in a DSC thermogram with a cooling ramp of 10° C./min from 220° C. to −90° C.; A vitrification around 35-45° C., as measured in a DSC thermogram with a cooling ramp of 10° C./min from 220° C. to −90° C.; A vitrification around 35, 36, 37, 38, 39, 40, 41, 42, 43, 44 or 45° C., as measured in a DSC thermogram with a cooling ramp of 10° C./min from 220° C. to −90° C.; A glass transition around 478° C., as measured in a DSC thermogram with a cooling ramp of 10° C./min from 220° C. to −90° C.; A glass transition around 42-52° C., as measured in a DSC thermogram with a cooling ramp of 10° C./min from 220° C. to −90° C.; and/or A glass transition around 42, 43, 44, 45, 46, 47, 48, 49, 51, or 52° C., as measured in a DSC thermogram with a cooling ramp of 10° C./min from 220° C. to −90° C.

In an embodiment, the 5-MeO-DMT salt is the phosphate salt. In an embodiment, the 5-MeO-DMT phosphate is crystalline. 5-MeO-DMT phosphate is not believed to display polymorphism, with the same crystalline XRPD pattern displayed for three solids isolated from different solvents. In an embodiment, crystalline 5-MeO-DMT phosphate is characterised by one or more peaks in an XRPD diffractogram as detailed in the Table below:

### XRPD Peak Data for Phosphate Pattern 1.

| | | | Rel. |
|---|---|---|---|
| Peak No. | Angle 2 θ | d Value | Intensity |
| 1 | 6.012° | 14.690 | 0.296 |
| 2 | 6.529° | 13.527 | 0.961 |
| 3 | 9.045° | 9.769 | 0.142 |
| 4 | 11.855° | 7.459 | 0.061 |
| 5 | 12.888° | 6.863 | 1.000 |
| 6 | 14.357° | 6.165 | 0.404 |
| 7 | 16.929° | 5.233 | 0.042 |
| 8 | 18.004° | 4.923 | 0.253 |
| 9 | 18.809° | 4.714 | 0.087 |
| 10 | 19.319° | 4.591 | 0.748 |
| 11 | 20.353° | 4.360 | 0.922 |
| 12 | 23.104° | 3.847 | 0.795 |
| 13 | 24.607° | 3.615 | 0.012 |
| 14 | 25.343° | 3.512 | 0.068 |
| 15 | 26.625° | 3.345 | 0.036 |
| 16 | 27.128° | 3.284 | 0.025 |
| 17 | 27.982° | 3.186 | 0.018 |
| 18 | 28.784° | 3.099 | 0.039 |
| 19 | 30.815° | 2.899 | 0.032 |
| 20 | 31.505° | 2.837 | 0.016 |

The person skilled in the art will appreciate that numerical values expressed herein to 3 decimal places may also be represented to 2 decimal or 1 decimal places. For example: 31.505° may be considered to be 31.51° or 31.5°.

In an embodiment, the crystalline 5-MeO-DMT phosphate is characterised by one or more of: One or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty peaks in an XRPD diffractogram as detailed in the Table above; One or more, two or more, three or more, four or more, five or more peaks in an XRPD diffractogram with a relative intensity of over 0.1, 0.2, 0.3, 0.4, 0.5, 0.6, 0.7, 0.8 or 0.9 as detailed in the Table above; A weight loss of 2.8% between ambient temperature and 60° C., as measured by TGA thermogram; A weight loss of between 1.5 to 3.5% between ambient temperature and 60° C., as measured by TGA thermogram; A weight loss of 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4 or 3.5% between ambient temperature and 60° C., as measured by TGA thermogram; A weight loss of 4.9% between 60 to 130° C., as measured by TGA thermogram; A weight loss of between 3.5 to 6.5% between 60 to 130° C., as measured by TGA thermogram; A weight loss of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4 or 6.5% between 6° and 130° C., as measured by TGA thermogram; A melting endothermic event with an onset of around 90.1° C. and a heat of fusion of 163.6 J/g, as measured in a DSC thermogram; A melting endothermic event with an onset of around 85 to 95° C. and a heat of fusion of around 155 to 170 J/g, as measured in a DSC thermogram; A melting endothermic event with an onset of around 85, 86, 87, 88, 89, 90, 91, 92, 93, 94 or 95° C. and a heat of fusion of around 155, 156, 157, 158, 159, 160, 161, 162, 163, 164, 165, 166, 167, 168, 169 or 170 J/g, as measured in a DSC thermogram; A vitrification around 71.7° C., as measured in a DSC thermogram with a cooling ramp of 10° C./min from 215° C. to −90° C.; A vitrification around 65-75° C., as measured in a DSC thermogram with a cooling ramp of 10° C./min from 215° C. to −90° C.; A vitrification around 65, 66, 67, 68, 69, 70, 71, 72, 73, 74 or 75° C., as measured in a DSC thermogram with a cooling ramp of 10° C./min from 215° C. to −90° C.; A glass transition around 75.1° C., as measured in a DSC thermogram with a cooling ramp of 10° C./min from 215° C. to −90° C.; A glass transition around 70-80° C., as measured in a DSC thermogram with a cooling ramp of 10° C./min from 215° C. to −90° C.; and/or A glass transition around 70, 71, 72, 73, 74, 75, 76, 77, 78, 79 or 80° C., as measured in a DSC thermogram with a cooling ramp of 10° C./min from 215° C. to −90° C.

In an embodiment, the 5-MeO-DMT salt is the fumarate salt. In an embodiment, the 5-MeO-DMT salt is crystalline. In an embodiment, the crystalline 5-MeO-DMT fumarate salt is characterised by one or more peaks in an XRPD diffractogram as detailed in the Table below:

### XRPD Data Peak for Fumarate Pattern 1.

| | | | Rel. |
|---|---|---|---|
| Peak No. | Angle 2 θ | d Value | Intensity |
| 1 | 5.911° | 14.940 | 0.015 |
| 2 | 6.459° | 13.673 | 0.073 |
| 3 | 10.694° | 8.266 | 0.012 |
| 4 | 12.107° | 7.305 | 0.104 |
| 5 | 12.981° | 6.815 | 1.000 |
| 6 | 14.203° | 6.231 | 0.021 |
| 7 | 14.975° | 5.911 | 0.034 |
| 8 | 16.304° | 5.432 | 0.673 |
| 9 | 17.532° | 5.055 | 0.233 |
| 10 | 18.166° | 4.879 | 0.498 |
| 11 | 19.220° | 4.614 | 0.604 |
| 12 | 19.448° | 4.561 | 0.345 |
| 13 | 20.396° | 4.351 | 0.598 |
| 14 | 20.783° | 4.271 | 0.073 |
| 15 | 21.487° | 4.132 | 0.037 |
| 16 | 22.052° | 4.028 | 0.859 |
| 17 | 22.834° | 3.891 | 0.523 |
| 18 | 24.255° | 3.667 | 0.015 |
| 19 | 25.064° | 3.550 | 0.243 |
| 20 | 25.410° | 3.502 | 0.310 |
| 21 | 26.116° | 3.409 | 0.200 |
| 22 | 27.542° | 3.236 | 0.135 |
| 23 | 28.051° | 3.178 | 0.174 |
| 24 | 28.709° | 3.107 | 0.035 |
| 25 | 30.166° | 2.960 | 0.071 |
| 26 | 30.482° | 2.930 | 0.024 |
| 27 | 30.764° | 2.904 | 0.031 |
| 28 | 31.022° | 2.880 | 0.034 |
| 29 | 31.555° | 2.833 | 0.008 |

In an embodiment, the crystalline 5-MeO-DMT fumarate salt is characterised by one or more of: One or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, twenty three or more, twenty four or more, twenty five or more, twenty six or more, twenty seven or more, twenty eight or more, or twenty nine peaks in an XRPD diffractogram as detailed in the Table above; One or more, two or more, three or more, four or more or five or more peaks in an XRPD diffractogram with a relative intensity of over 0.1, 0.2, 0.3, 0.4, 0.5, 0.6, 0.7, 0.8 or 0.9 as detailed in the Table above; Two small endothermic events around 93.7° C. and 134.0° C. respectively as measured in a DSC thermogram; Two small endothermic events around 85-100° C. and 130-140° C. respectively as measured in a DSC thermogram; Two small endothermic events around 85, 86, 87, 88, 89, 90, 91, 92, 93, 94, 95, 96, 97, 98, 99 or 100° C. and 130, 131, 132, 133, 134, 135, 136, 137, 138, 139 or 140° C. respectively as measured in a DSC thermogram; A sharp endotherm with an onset of around 176.5° C. and heat of fusion 92.3 J/g as measured in a DSC thermogram; A sharp endotherm with an onset of around 165-185° C. and heat of fusion about 88-100 J/g as measured in a DSC thermogram; A sharp endotherm with an onset of around 165, 166, 167, 168, 169, 170, 171, 172, 173, 174, 175, 176, 177, 178, 179, 180, 181, 182, 183, 184, or 185° C. and heat of fusion about 88, 89, 90, 91, 92, 93, 94, 95, 96, 97, 98, 99, or 100 J/g as measured in a DSC thermogram; A vitrification around 45.1° C., as measured in a DSC thermogram with a cooling ramp of 10° C./min from 215° C. to −90° C.; A vitrification around 40-50° C., as measured in a DSC thermogram with a cooling ramp of 10° C./min from 215° C. to −90° C.; A vitrification around 40, 41, 42, 43, 44, 45, 46, 47, 48, 49 or 50° C., as measured in a DSC thermogram with a cooling ramp of 10° C./min from 215° C. to −90° C.; A glass transition around 51.1° C., as measured in a DSC thermogram with a cooling ramp of 10° C./min from 215° C. to −90° C.; A glass transition around 45-55° C., as measured in a DSC thermogram with a cooling ramp of 10° C./min from 215° C. to −90° C.; and/or A glass transition around 45, 46, 47, 48, 49, 50, 51, 52, 53, 54 or 55° C., as measured in a DSC thermogram with a cooling ramp of 10° C./min from 215° C. to −90° C.

In an embodiment, the 5-MeO-DMT salt is the oxalate salt. In an embodiment, the 5-MeO-DMT salt is crystalline. In an embodiment, the crystalline 5-MeO-DMT oxalate salt is characterised by one or more peaks in an XRPD diffractogram as detailed in the Table below:

### XRPD Peak Data for Oxalate Pattern 1.

| | | | Rel. |
|---|---|---|---|
| Peak No. | Angle 2 θ | d Value | Intensity |
| 1 | 7.923° | 11.150 | 0.062 |
| 2 | 10.999° | 8.037 | 0.012 |
| 3 | 11.688° | 7.565 | 0.002 |
| 4 | 12.963° | 6.824 | 1.000 |
| 5 | 13.697° | 6.460 | 0.095 |
| 6 | 14.779° | 5.989 | 0.005 |
| 7 | 16.600° | 5.336 | 0.001 |
| 8 | 17.217° | 5.146 | 0.002 |
| 9 | 17.777° | 4.985 | 0.004 |
| 10 | 18.284° | 4.848 | 0.001 |
| 11 | 18.570° | 4.774 | 0.010 |
| 12 | 18.861° | 4.701 | 0.003 |
| 13 | 20.147° | 4.404 | 0.005 |
| 14 | 19.928° | 4.452 | 0.214 |
| 15 | 21.360° | 4.157 | 0.004 |
| 16 | 21.967° | 4.043 | 0.100 |
| 17 | 23.560° | 3.773 | 0.077 |
| 18 | 23.933° | 3.715 | 0.003 |
| 19 | 24.348° | 3.653 | 0.003 |
| 20 | 25.159° | 3.537 | 0.002 |
| 21 | 25.573° | 3.481 | 0.010 |
| 22 | 25.961° | 3.429 | 0.377 |
| 23 | 26.348° | 3.380 | 0.015 |
| 24 | 26.605° | 3.348 | 0.001 |
| 25 | 27.458° | 3.246 | 0.080 |
| 26 | 27.960° | 3.189 | 0.001 |
| 27 | 29.719° | 3.004 | 0.011 |
| 28 | 30.525° | 2.926 | 0.005 |
| 29 | 31.562° | 2.832 | 0.015 |

In an embodiment, the crystalline 5-MeO-DMT oxalate salt is characterised by one or more of: One or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, twenty three or more, twenty four or more, twenty five or more, twenty six or more, twenty seven or more, twenty eight or more, or twenty nine peaks in an XRPD diffractogram as detailed in the Table above; One or more, two or more, three or more, four or more or five or more peaks in an XRPD diffractogram with a relative intensity of over 0.1, 0.2, 0.3, 0.4, 0.5, 0.6, 0.7, 0.8 or 0.9 as detailed in the Table above; A weight loss of around 0.6% between 25-180° C., as measured by TGA thermogram; A weight loss of around 0.1-1.0% between 25-180° C., as measured by TGA thermogram; A weight loss of around 0.1, 0.2, 0.3, 0.4, 0.5, 0.6, 0.7, 0.8, 0.9 or 1.0% between 25-180° C., as measured by TGA thermogram; A melting endothermic event with an onset of around 176.1° C. and an enthalpy of 157.5 J/g, as measured in a DSC thermogram; A melting endothermic event with an onset of around 170-180° C. and an enthalpy of around 152-162 J/g, as measured in a DSC thermogram; A melting endothermic event with an onset of around 170, 171, 172, 173, 174, 175, 176, 177, 178, 179 or 180° C. and an enthalpy of around 152, 153, 154, 155, 156, 157, 158, 159, 160, 161 or 162 J/g, as measured in a DSC thermogram; A vitrification around 50.7° C., as measured in a DSC thermogram with a cooling ramp of 10° C./min from 215° C. to −90° C.; A vitrification around 45-55° C., as measured in a DSC thermogram with a cooling ramp of 10° C./min from 215° C. to −90° C.; A vitrification around 45, 46, 47, 48, 49, 50, 51, 52, 53, 54 or 55° C., as measured in a DSC thermogram with a cooling ramp of 10° C./min from 215° C. to −90° C.; A glass transition around 58.0° C., as measured in a DSC thermogram with a cooling ramp of 10° C./min from 215° C. to −90° C.; A glass transition around 53-63° C., as measured in a DSC thermogram with a cooling ramp of 10° C./min from 215° C. to −90° C.; and/or A glass transition around 53, 54, 55, 56, 57, 58, 59, 60, 61, 62 or 63° C., as measured in a DSC thermogram with a cooling ramp of 10° C./min from 215° C. to −90° C.

In an embodiment, the 5-MeO-DMT salt for the adipate salt. In an embodiment, the 5-MeO-DMT salt is crystalline. In an embodiment, the crystalline 5-MeO-DMT adipate salt is characterised by one or more peaks in an XRPD diffractogram as detailed in the Table below:

### XRPD Peak Data for Adipate, Pattern 2.

| | | | Rel. |
|---|---|---|---|
| Peak No. | Angle 2 θ | d Value | Intensity |
| 1 | 9.368° | 9.433 | 1.000 |
| 2 | 14.136° | 6.260 | 0.493 |
| 3 | 14.535° | 6.089 | 0.039 |
| 4 | 17.694° | 5.009 | 0.611 |
| 5 | 18.017° | 4.919 | 0.448 |
| 6 | 18.692° | 4.743 | 0.052 |
| 7 | 19.490° | 4.551 | 0.414 |
| 8 | 20.846° | 4.258 | 0.309 |
| 9 | 21.557° | 4.119 | 0.338 |
| 10 | 21.831° | 4.068 | 0.433 |
| 11 | 23.433° | 3.793 | 0.368 |
| 12 | 24.948° | 3.566 | 0.027 |
| 13 | 25.633° | 3.473 | 0.117 |
| 14 | 25.831° | 3.446 | 0.308 |
| 15 | 26.875° | 3.315 | 0.157 |
| 16 | 28.213° | 3.161 | 0.214 |
| 17 | 28.503° | 3.129 | 0.058 |
| 18 | 30.224° | 2.955 | 0.079 |
| 19 | 30.499° | 2.929 | 0.052 |
| 20 | 31.203° | 2.864 | 0.054 |
| 21 | 31.867° | 2.806 | 0.033 |

In an embodiment, the crystalline 5-MeO-DMT adipate salt is characterised by one or more of: One or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, or twenty one peaks in an XRPD diffractogram as detailed in the Table above; One or more, two or more, three or more, four or more, or five or more peaks in an XRPD diffractogram with a relative intensity of over 0.1, 0.2, 0.3, 0.4, 0.5, 0.6, 0.7, 0.8 or 0.9 as detailed in the Table above; A weight loss of 0.5% between 25-170° C., as measured by TGA thermogram; A weight loss of around 0.1-1.0% between 25-170° C., as measured by TGA thermogram; A weight loss of around 0.1, 0.2, 0.3, 0.4, 0.5, 0.6, 0.7, 0.8, 0.9 or 1.0% between 25-170° C., as measured by TGA thermogram; A melting endothermic event with an onset of around 73.9° C. and an enthalpy of 90.9 J/g, as measured in a DSC thermogram; A melting endothermic event with an onset of around 68-80° C. and an enthalpy of around 85-95 J/g, as measured in a DSC thermogram; A melting endothermic event with an onset of around 68, 69, 70, 71, 72, 73, 74, 75, 76, 77, 78, 79 or 80° C. and an enthalpy of around 85, 86, 87, 88, 89, 90, 91, 92, 93, 94 or 95 J/g, as measured in a DSC thermogram; A vitrification around 3.1° C., as measured in a DSC thermogram with a cooling ramp of 10° C./min from 215° C. to −90° C.; A vitrification around 0-10° C., as measured in a DSC thermogram with a cooling ramp of 10° C./min from 215° C. to −90° C.; A vitrification around 0, 1, 2, 3, 4, 5, 6, 7, 8, 9 or 10° C., as measured in a DSC thermogram with a cooling ramp of 10° C./min from 215° C. to −90° C.; A glass transition around 7.4° C., as measured in a DSC thermogram with a cooling ramp of 10° C./min from 215° C. to −90° C.; A glass transition around 2-12° C., as measured in a DSC thermogram with a cooling ramp of 10° C./min from 215° C. to −90° C.; and/or A glass transition around 2, 3, 4, 5, 6, 7, 8, 9, 10, 11 or 12° C., as measured in a DSC thermogram with a cooling ramp of 10° C./min from 215° C. to −90° C.

In an embodiment, the 5-MeO-DMT salt is the tartrate salt. In an embodiment, the 5-MeO-DMT salt is crystalline. In an embodiment, the crystalline 5-MeO-DMT tartrate salt is characterised by one or more peaks in an XRPD diffractogram as detailed in the Table below:

### XRPD Peak Data for Tartrate Pattern 1.

| | | | Rel. |
|---|---|---|---|
| Peak No. | Angle 2 θ | d Value | Intensity |
| 1 | 10.147° | 8.711 | 0.045 |
| 2 | 10.987° | 8.046 | 0.012 |
| 3 | 11.794° | 7.497 | 0.050 |
| 4 | 13.080° | 6.763 | 0.246 |
| 5 | 14.786° | 5.986 | 0.015 |
| 6 | 15.214° | 5.819 | 0.331 |
| 7 | 15.464° | 5.725 | 0.093 |
| 8 | 16.534° | 5.357 | 0.138 |
| 9 | 17.793° | 4.981 | 0.374 |
| 10 | 18.267° | 4.853 | 0.692 |
| 11 | 18.579° | 4.772 | 0.687 |
| 12 | 18.785° | 4.720 | 0.684 |
| 13 | 19.360° | 4.581 | 0.044 |
| 14 | 19.630° | 4.519 | 0.187 |
| 15 | 20.311° | 4.369 | 0.683 |
| 16 | 20.730° | 4.281 | 1.000 |
| 17 | 21.467° | 4.136 | 0.161 |
| 18 | 22.125° | 4.015 | 0.461 |
| 19 | 22.639° | 3.924 | 0.179 |
| 20 | 23.041° | 3.857 | 0.114 |
| 21 | 23.679° | 3.754 | 0.258 |
| 22 | 24.856° | 3.579 | 0.079 |
| 23 | 25.431° | 3.500 | 0.135 |
| 24 | 26.034° | 3.420 | 0.749 |
| 25 | 26.368° | 3.377 | 0.042 |
| 26 | 27.029° | 3.296 | 0.068 |
| 27 | 27.299° | 3.264 | 0.130 |
| 28 | 27.870° | 3.199 | 0.064 |
| 29 | 28.937° | 3.083 | 0.024 |
| 30 | 29.311° | 3.045 | 0.088 |
| 31 | 30.009° | 2.975 | 0.121 |
| 32 | 31.153° | 2.869 | 0.067 |
| 33 | 31.707° | 2.820 | 0.047 |

In an embodiment, the crystalline 5-MeO-DMT tartrate salt is characterised by one or more of One or more, two or more, three or more, four or more or five or more peaks in an XRPD diffractogram with a relative intensity of over 0.1, 0.2, 0.3, 0.4, 0.5, 0.6, 0.7, 0.8 or 0.9 as detailed in the Table above; A weight loss of 1% between 25-170° C., as measured by TGA thermogram; A weight loss of around 0.1-1.0% between 25-170° C., as measured by TGA thermogram; A weight loss of around 0.1, 0.2, 0.3, 0.4, 0.5, 0.6, 0.7, 0.8, 0.9 or 1.0% between 25-170° C., as measured by TGA thermogram; A melting endothermic event with an onset of around 138.9° C. and an enthalpy of 97.0 J/g, as measured in a DSC thermogram; A melting endothermic event with an onset of 0 around 130-145° C. and an enthalpy of around 92-102 J/g, as measured in a DSC thermogram; A melting endothermic event with an onset of around 130, 131, 132, 133, 134, 135, 136, 137, 138, 139, 140, 141, 142, 143, 144 or 145° C. and an enthalpy of around 92, 93, 94, 95, 96, 97, 98, 99, 100, 101 or 102 J/g, as measured in a DSC thermogram; A vitrification around 49° C., as measured in a DSC thermogram with a cooling ramp of 10° C./min from 200° C. to −90° C.; A vitrification around 45-55° C., as measured in a DSC thermogram with a cooling ramp of 10° C./min from 200° C. to −90° C.; A vitrification around 45, 46, 47, 48, 49, 50, 51, 52, 53, 54 or 55° C., as measured in a DSC thermogram with a cooling ramp of 10° C./min from 200° C. to −90° C.; A glass transition around 54.2° C., as measured in a DSC thermogram with a cooling ramp of 10° C./min from 200° C. to −90° C.; A glass transition around 50-60° C., as measured in a DSC thermogram with a cooling ramp of 10° C./min from 200° C. to −90° C.; A glass transition around 50, 51, 52, 53, 54, 55, 56, 57, 58, 59 or 60° C., as measured in a DSC thermogram with a cooling ramp of 10° C./min from 200° C. to −90° C.; and/or A total water uptake between 0% RH and 90% RH at 25° C. of approximately 3.3% w/w.

In an embodiment, the 5-MeO-DMT salt is the tosylate salt. In an embodiment, the 5-MeO-DMT salt is crystalline. In an embodiment, the crystalline 5-MeO-DMT tosylate salt is characterised by one or more peaks in an XRPD diffractogram as detailed in the Table below:

### XRPD Peak Data for Tosylate Pattern 1.

| | | | Rel. |
|---|---|---|---|
| Peak No. | Angle 2 θ | d Value | Intensity |
| 1 | 9.609° | 9.197 | 0.296 |
| 2 | 12.001° | 7.369 | 0.041 |
| 3 | 12.934° | 6.839 | 0.007 |
| 4 | 13.798° | 6.413 | 0.386 |
| 5 | 14.391° | 6.150 | 0.315 |
| 6 | 16.105° | 5.499 | 0.034 |
| 7 | 16.625° | 5.328 | 0.138 |
| 8 | 17.489° | 5.067 | 0.157 |
| 9 | 18.114° | 4.893 | 0.142 |
| 10 | 19.342° | 4.585 | 0.622 |
| 11 | 19.945° | 4.448 | 0.119 |
| 12 | 22.076° | 4.023 | 0.034 |
| 13 | 23.161° | 3.837 | 0.057 |
| 14 | 23.571° | 3.771 | 0.472 |
| 15 | 24.054° | 3.697 | 1.000 |
| 16 | 25.037° | 3.554 | 0.130 |
| 17 | 25.979° | 3.427 | 0.023 |
| 18 | 26.435° | 3.369 | 0.107 |
| 19 | 27.302° | 3.264 | 0.420 |
| 20 | 27.712° | 3.216 | 0.097 |
| 21 | 28.880° | 3.089 | 0.123 |
| 22 | 29.371° | 3.039 | 0.013 |
| 23 | 29.769° | 2.999 | 0.026 |
| 24 | 30.161° | 2.961 | 0.046 |
| 25 | 30.981° | 2.884 | 0.032 |
| 26 | 31.738° | 2.817 | 0.048 |

In an embodiment, the crystalline 5-MeO-DMT tosylate salt is characterised by one or more of: One or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, twenty three or more, twenty four or more, twenty five or more, or twenty six peaks in an XRPD diffractogram as detailed in the Table above; One or more, two or more, three or more, four or more or five or more peaks in an XRPD diffractogram with a relative intensity of over 0.1, 0.2, 0.3, 0.4, 0.5, 0.6, 0.7, 0.8 or 0.9 as detailed in the Table above; A weight loss of 1.0% between 25-230° C., as measured by TGA thermogram; A weight loss of around 0.5-1.5% between 25-230° C., as measured by TGA thermogram; A weight loss of around 0.5, 0.6, 0.7, 0.8, 0.9, 1.0, 1.1, 1.2, 1.3, 1.4 or 1.5% between 25-230° C., as measured by TGA thermogram; A melting endothermic event with an onset of around 109.7° C. and an enthalpy of 89.3 J/g, as measured in a DSC thermogram; A melting endothermic event with an onset of around 105-115° C. and an enthalpy of around 85-95 J/g, as measured in a DSC thermogram; A melting endothermic event with an onset of around 105, 106, 107, 108, 109, 110, 111, 112, 113, 114 or 115° C. and an enthalpy of around 85, 86, 87, 88, 89, 90, 91, 92, 93, 94 or 95 J/g, as measured in a DSC thermogram; A vitrification around 24.3° C., as measured in a DSC thermogram with a cooling ramp of 10° C./min from 230° C. to −90° C.; A vitrification around 20-30° C., as measured in a DSC thermogram with a cooling ramp of 10° C./min from 230° C. to −90° C.; A vitrification around 20, 21, 22, 23, 24, 25, 26, 27, 28, 29 or 30° C., as measured in a DSC thermogram with a cooling ramp of 10° C./min from 230° C. to −90° C.; A glass transition around 30.2° C., as measured in a DSC thermogram with a cooling ramp of 10° C./min from 230° C. to −90° C.; A glass transition around 25-35° C., as measured in a DSC thermogram with a cooling ramp of 10° C./min from 230° C. to −90° C.; A glass transition around 25, 26, 27, 28, 29, 30, 31, 32, 33, 34 or 35° C., as measured in a DSC thermogram with a cooling ramp of 10° C./min from 230° C. to −90° C.; A total water uptake between 0% RH and 90% RH at 25° C. of approximately 1.9% w/w; A total water uptake between 0% RH and 90% RH at 25° C. of approximately 1.5-2.5% w/w; and/or A total water uptake between 0% RH and 90% RH at 25° C. of approximately 1.5, 1.6, 1.7, 1.8, 1.9, 2.0, 2.1, 2.2, 2.3, 2.4 or 2.5% w/w.

In an embodiment, the 5-MeO-DMT salt is the benzenesulfonate salt. In an embodiment, the 5-MeO-DMT salt is crystalline. In an embodiment, the crystalline 5-MeO-DMT benzenesulfonate salt is characterised by one or more peaks in an XRPD diffractogram as detailed in the Table below:

### XRPD Peak Data for Benzenesulfonate Pattern 1.

| | | | Rel. |
|---|---|---|---|
| Peak No. | Angle 2 θ | d Value | Intensity |
| 1 | 8.579° | 10.298 | 0.006 |
| 2 | 9.490° | 9.312 | 1.000 |
| 3 | 9.738° | 9.076 | 0.051 |
| 4 | 12.189° | 7.255 | 0.006 |
| 5 | 13.449° | 6.578 | 0.064 |
| 6 | 14.164° | 6.248 | 0.260 |
| 7 | 14.703° | 6.020 | 0.225 |
| 8 | 14.997° | 5.903 | 0.315 |
| 9 | 16.791° | 5.276 | 0.105 |
| 10 | 17.770° | 4.987 | 0.213 |
| 11 | 18.035° | 4.915 | 0.393 |
| 12 | 18.506° | 4.791 | 0.025 |
| 13 | 18.872° | 4.698 | 0.266 |
| 14 | 20.035° | 4.428 | 0.024 |
| 15 | 20.332° | 4.364 | 0.092 |
| 16 | 21.215° | 4.185 | 0.535 |
| 17 | 21.954° | 4.045 | 0.014 |
| 18 | 22.526° | 3.944 | 0.025 |
| 19 | 22.964° | 3.870 | 0.023 |
| 20 | 23.338° | 3.809 | 0.175 |
| 21 | 23.571° | 3.771 | 0.704 |
| 22 | 23.911° | 3.719 | 0.039 |
| 23 | 24.435° | 3.640 | 0.389 |
| 24 | 24.655° | 3.608 | 0.331 |
| 25 | 25.050° | 3.552 | 0.188 |
| 26 | 26.152° | 3.405 | 0.104 |
| 27 | 26.536° | 3.356 | 0.083 |
| 28 | 26.962° | 3.304 | 0.015 |
| 29 | 27.316° | 3.262 | 0.017 |
| 30 | 28.333° | 3.147 | 0.295 |
| 31 | 28.900° | 3.087 | 0.010 |
| 32 | 29.249° | 3.051 | 0.076 |
| 33 | 29.621° | 3.013 | 0.007 |
| 34 | 30.064° | 2.970 | 0.013 |
| 35 | 30.564° | 2.923 | 0.011 |
| 36 | 31.017° | 2.881 | 0.010 |
| 37 | 31.901° | 2.803 | 0.054 |

In an embodiment, the crystalline 5-MeO-DMT benzenesulfonate salt is characterised by one or more of: One or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, twenty three or more, twenty four or more, twenty five or more, twenty six or more, twenty seven or more, twenty eight or more, twenty nine or more, thirty or more, thirty one or more, thirty two or more, thirty three or more, thirty four or more, thirty five or more, thirty six or more, or thirty seven peaks in an XRPD diffractogram as detailed in the Table above; One or more, two or more, three or more, four or more or five or more peaks in an XRPD diffractogram with a relative intensity of over 0.1, 0.2, 0.3, 0.4, 0.5, 0.6, 0.7, 0.8 or 0.9 as detailed in the Table above; A weight loss of 1.5% between 25-250° C., as measured by TGA thermogram; A weight loss of around 1.0-2.0% between 25-250° C., as measured by TGA thermogram; A weight loss of around 1.0, 1.1, 1.2, 1.3, 1.4, 1.5, 1.6, 1.7, 1.8, 1.9 or 2.0% between 25-250° C., as measured by TGA thermogram; A melting endothermic event with an onset of around 76.2° C. and an enthalpy of 66.5 J/g, as measured in a DSC thermogram; A melting endothermic event with an onset of around 70-80° C. and an enthalpy of around 60-70 J/g, as measured in a DSC thermogram; A melting endothermic event with an onset of around 70, 71, 72, 73, 74, 75, 76, 77, 78, 79 or 80° C. and an enthalpy of around 60, 61, 62, 63, 64, 65, 66, 67, 68, 69 or 70 J/g, as measured in a DSC thermogram; A weak exothermic event at around 140° C. as measured in a DSC thermogram; A weak exothermic event at around 135-145° C. as measured in a DSC thermogram; A weak exothermic event at around 135, 136, 137, 138, 139, 140, 141, 142, 143, 144 or 145° C. as measured in a DSC thermogram; A vitrification around 17.7° C., as measured in a DSC thermogram with a cooling ramp of 10° C./min from 230° C. to −90° C.; A vitrification around 12-22° C., as measured in a DSC thermogram with a cooling ramp of 10° C./min from 230° C. to −90° C.; A vitrification around 12, 13, 14, 15, 16, 17, 18, 19, 20, 21 or 22° C., as measured in a DSC thermogram with a cooling ramp of 10° C./min from 230° C. to −90° C.; A glass transition around 23.3° C., as measured in a DSC thermogram with a cooling ramp of 10° C./min from 230° C. to −90° C.; A glass transition around 18-28° C., as measured in a DSC thermogram with a cooling ramp of 10° C./min from 230° C. to −90° C.; and/or A glass transition around 18, 19, 20, 21, 22, 23, 24, 25, 26, 27 or 28° C., as measured in a DSC thermogram with a cooling ramp of 10° C./min from 230° C. to −90° C.

In an embodiment, the 5-MeO-DMT salt is the glycolate salt. In an embodiment, the 5-MeO-DMT salt is crystalline. In an embodiment, the crystalline 5-MeO-DMT glycolate salt is characterised by one or more peaks in an XRPD diffractogram as detailed in the Table below:

### XRPD Peak Data for Glycolate Pattern 1.

| | | | Rel. |
|---|---|---|---|
| Peak No. | Angle 2 θ | d Value | Intensity |
| 1 | 8.837° | 9.999 | 0.004 |
| 2 | 10.114° | 8.739 | 0.551 |
| 3 | 11.674° | 7.574 | 0.255 |
| 4 | 15.436° | 5.736 | 0.063 |
| 5 | 16.557° | 5.350 | 0.011 |
| 6 | 17.187° | 5.155 | 0.107 |
| 7 | 17.923° | 4.945 | 0.047 |
| 8 | 19.656° | 4.513 | 0.025 |
| 9 | 20.235° | 4.385 | 1.000 |
| 10 | 21.085° | 4.210 | 0.624 |
| 11 | 21.908° | 4.054 | 0.034 |
| 12 | 22.401° | 3.966 | 0.088 |
| 13 | 22.979° | 3.867 | 0.073 |
| 14 | 23.408° | 3.797 | 0.923 |
| 15 | 24.340° | 3.654 | 0.364 |
| 16 | 24.944° | 3.567 | 0.011 |
| 17 | 25.495° | 3.491 | 0.081 |
| 18 | 26.873° | 3.315 | 0.079 |
| 19 | 27.449° | 3.247 | 0.169 |
| 20 | 27.781° | 3.209 | 0.012 |
| 21 | 28.878° | 3.089 | 0.015 |
| 22 | 29.704° | 3.005 | 0.005 |
| 23 | 30.565° | 2.922 | 0.267 |
| 24 | 31.124° | 2.871 | 0.113 |
| 25 | 31.793° | 2.812 | 0.008 |

In an embodiment, the crystalline 5-MeO-DMT glycolate salt is characterised by one or more of One or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, twenty three or more, twenty four or more, or twenty five peaks in an XRPD diffractogram as detailed in the Table above; One or more, two or more, three or more, four or more or five or more peaks in an XRPD diffractogram with a relative intensity of over 0.1, 0.2, 0.3, 0.4, 0.5, 0.6, 0.7, 0.8 or 0.9 as detailed in the Table above; A weight loss of 0 about 1.4% between 25-155° C., as measured by TGA thermogram; A weight loss of about 0.9-1.9% between 25-155° C., as measured by TGA thermogram; A weight loss of about 0.9, 1.0, 1.1, 1.2, 1.3, 1.4, 1.5, 1.6, 1.7, 1.8 or 1.9% between 25-155° C., as measured by TGA thermogram; A melting endothermic event with an onset of around 95.2° C. and an enthalpy of 100.5 J/g, as measured in a DSC thermogram; A melting endothermic event with an onset of around 90-100° C. and an enthalpy of around 95-105 J/g, as measured in a DSC thermogram; A melting endothermic event with an onset of around 90, 91, 92, 93, 94, 95, 96, 97, 98, 99 or 100° C. and an enthalpy of around 95, 96, 97, 98, 99, 100, 101, 102, 103, 104 or 105 J/g, as measured in a DSC thermogram; A vitrification around 7.5° C., as measured in a DSC thermogram with a cooling ramp of 10° C./min from 200° C. to −90° C.; A vitrification around 2-12° C., as measured in a DSC thermogram with a cooling ramp of 10° C./min from 200° C. to −90° C.; A vitrification around 2, 3, 4, 5, 6, 7, 8, 9, 10, 11 or 12° C., as measured in a DSC thermogram with a cooling ramp of 10° C./min from 200° C. to −90° C.; A glass transition around 14.5° C., as measured in a DSC thermogram with a cooling ramp of 10° C./min from 200° C. to −90° C.; A glass transition around 10-20° C., as measured in a DSC thermogram with a cooling ramp of 10° C./min from 200° C. to −90° C.; and/or A glass transition around 10, 11, 12, 13, 14, 15, 16, 17, 18, 19 or 20° C., as measured in a DSC thermogram with a cooling ramp of 10° C./min from 200° C. to −90° C.

In an embodiment, the 5-MeO-DMT salt is the ketoglutarate salt. In an embodiment, the 5-MeO-DMT salt is crystalline. In an embodiment, the crystalline 5-MeO-DMT ketoglutarate salt is characterised by one or more peaks in an XRPD diffractogram as detailed in the Table below:

### XRPD Peak Data for Ketoglutarate Pattern 1.

| | | | Rel. |
|---|---|---|---|
| Peak No. | Angle 2 θ | d Value | Intensity |
| 1 | 9.964° | 8.870 | 0.098 |
| 2 | 11.503° | 7.687 | 0.056 |
| 3 | 14.386° | 6.152 | 0.348 |
| 4 | 15.796° | 5.606 | 0.020 |
| 5 | 17.613° | 5.031 | 0.116 |
| 6 | 18.157° | 4.882 | 1.000 |
| 7 | 18.922° | 4.686 | 0.185 |
| 8 | 19.940° | 4.449 | 0.053 |
| 9 | 20.460° | 4.337 | 0.039 |
| 10 | 20.914° | 4.244 | 0.399 |
| 11 | 21.420° | 4.145 | 0.057 |
| 12 | 22.517° | 3.946 | 0.263 |
| 13 | 23.165° | 3.836 | 0.208 |
| 14 | 23.861° | 3.726 | 0.202 |
| 15 | 24.534° | 3.625 | 0.010 |
| 16 | 25.616° | 3.475 | 0.337 |
| 17 | 26.071° | 3.415 | 0.083 |
| 18 | 26.670° | 3.340 | 0.130 |
| 19 | 27.361° | 3.257 | 0.091 |
| 20 | 27.700° | 3.218 | 0.115 |
| 21 | 28.957° | 3.081 | 0.016 |
| 22 | 29.518° | 3.024 | 0.026 |
| 23 | 30.025° | 2.974 | 0.042 |
| 24 | 30.580° | 2.921 | 0.035 |

In an embodiment, the crystalline 5-MeO-DMT ketoglutarate salt is characterised by one or more of One or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, twenty three or more, or twenty four peaks in an XRPD diffractogram as detailed in the Table above; One or more, two or more, three or more, four or more or five or more peaks in an XRPD diffractogram with a relative intensity of over 0.1, 0.2, 0.3, 0.4, 0.5, 0.6, 0.7, 0.8 or 0.9 as detailed in the Table above; A weight loss of 0 about 1.2% between 25-150° C., as measured by TGA thermogram; A weight loss of 3 about 0.7.1.74 between 25-150° C., as measured by TGA thermogram; A weight loss of about 0.7, 0.8, 0.9, 1.0, 1.1, 1.2, 1.3, 1.4, 1.5, 1.6 or 1.7% between 25-150° C., as measured by TGA thermogram; A melting endothermic event with an onset of around 85.5° C. and an enthalpy of 92.4 J/g, as measured in a DSC thermogram; A melting endothermic event with an onset of around 80-90° C. and an enthalpy of around 87-97 J/g, as measured in a DSC thermogram; A melting endothermic event with an onset of around 80, 81, 82, 83, 84, 85, 86, 87, 88, 89 or 90° C. and an enthalpy of around 87, 88, 89, 90, 91, 92, 93, 94, 95, 96 or 97 J/g, as measured in a DSC thermogram; A vitrification around 21.9° C., as measured in a DSC thermogram with a cooling ramp of 10° C./min from 150° C. to −90° C.; A vitrification around 16-26° C., as measured in a DSC thermogram with a cooling ramp of 10° C./min from 150° C. to −90° C.; A vitrification around 16, 17, 18, 19, 20, 21, 22, 23, 24, 25 or 26° C., as measured in a DSC thermogram with a cooling ramp of 10° C./min from 150° C. to −90° C.; A glass transition around 28.5° C., as measured in a DSC thermogram with a cooling ramp of 10° C./min from 150° C. to −90° C.; A glass transition around 23-33° C., as measured in a DSC thermogram with a cooling ramp of 10° C./min from 150° C. to −90° C.; A glass transition around 23, 24, 25, 26, 27, 28, 29, 30, 31, 32 or 33° C., as measured in a DSC thermogram with a cooling ramp of 10° C./min from 150° C. to −90° C.; A gradual water uptake between 403RH (0.13% w/w) to 801 RH (2.55 w/w), optionally with a rapid increase to 28.140 w/w at 90% RH; and/or A gradual water uptake between 40% RH (0.05-0.2% w/w) to 80% RH (1.50-3.5% w/w), optionally with a rapid increase to 20-40% w/w at 90% RH. In an embodiment, the 5-MeO-DMT salt is the malate salt. In an embodiment, the 5-MeO-DMT salt is crystalline. In an embodiment, the crystalline 5-MeO-DMT malate salt is characterised by one or more peaks in an XRPD diffractogram as detailed in the Table below:

### XRPD Peak Data for Malate Pattern 1

| | | | Rel. |
|---|---|---|---|
| Peak No. | Angle 2 θ | d Value | Intensity |
| 1 | 11.823° | 7.479 | 0.136 |
| 2 | 14.733° | 6.008 | 0.392 |
| 3 | 15.588° | 5.680 | 0.058 |
| 4 | 18.254° | 4.856 | 1.000 |
| 5 | 18.666° | 4.750 | 0.746 |
| 6 | 18.889° | 4.694 | 0.968 |
| 7 | 20.330° | 4.365 | 0.035 |
| 8 | 20.782° | 4.271 | 0.025 |
| 9 | 21.080° | 4.211 | 0.052 |
| 10 | 21.608° | 4.109 | 0.475 |
| 11 | 21.963° | 4.044 | 0.335 |
| 12 | 23.014° | 3.861 | 0.068 |
| 13 | 23.662° | 3.757 | 0.456 |
| 14 | 24.119° | 3.687 | 0.474 |
| 15 | 26.084° | 3.413 | 0.505 |
| 16 | 26.608° | 3.347 | 0.168 |
| 17 | 27.179° | 3.278 | 0.131 |
| 18 | 27.839° | 3.202 | 0.210 |
| 19 | 29.242° | 3.052 | 0.011 |
| 20 | 29.649° | 3.011 | 0.022 |
| 21 | 30.113° | 2.965 | 0.113 |
| 22 | 30.700° | 2.910 | 0.046 |
| 23 | 31.219° | 2.863 | 0.024 |
| 24 | 31.821° | 2.810 | 0.056 |

In an embodiment, the crystalline 5-MeO-DMT malate salt is characterised by one or more of: One or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, twenty three or more, or twenty four peaks in an XRPD diffractogram as detailed in the Table above; One or more, two or more, three or more, four or more or five or more peaks in an XRPD diffractogram with a relative intensity of over 0.1, 0.2, 0.3, 0.4, 0.5, 0.6, 0.7, 0.8 or 0.9 as detailed in the Table above; A weight loss of about 2.6% between ambient temperature and 170° C., as measured by TGA thermogram; A weight loss of about 2.0-3.0% between ambient temperature and 170° C., as measured by TGA thermogram; A weight loss of about 2.0, 2.1, 2.2, 2.3, 2.4, 2.5, 2.6, 2.7, 2.8, 2.9 or 3.0% between ambient temperature and 170° C., as measured by TGA thermogram; A melting endothermic event with an onset of around 80.9° C. and an enthalpy of 87.0 J/g, as measured in a DSC thermogram; A melting endothermic event with an onset of around 75-85° C. and an enthalpy of around 82-92 J/g, as measured in a DSC thermogram; A melting endothermic event with an onset of around 75, 76, 77, 78, 79, 80, 81, 82, 83, 84 or 85° C. and an enthalpy of around 82, 83, 84, 85, 86, 87, 88, 89, 90, 91 or 92 J/g, as measured in a DSC thermogram; A vitrification around 15.1° C., as measured in a DSC thermogram with a cooling ramp of 10° C./min from 170° C. to −90° C.; A vitrification around 10-20° C., as measured in a DSC thermogram with a cooling ramp of 10° C./min from 170° C. to −90° C.; A vitrification around 10, 11, 12, 13, 14, 15, 16, 17, 18, 19 or 20° C., as measured in a DSC thermogram with a cooling ramp of 10° C./min from 170° C. to −90° C.; A glass transition around 22° C., as measured in a DSC thermogram with a cooling ramp of 10° C./min from 170° C. to −90° C.; A glass transition around 17-27° C., as measured in a DSC thermogram with a cooling ramp of 10° C./min from 170° C. to −90° C.; and/or A glass transition around 17, 18, 19, 20, 21, 22, 23, 24, 25, 26 or 27° C., as measured in a DSC thermogram with a cooling ramp of 10° C./min from 170° C. to −90° C.

In an embodiment, the 5-MeO-DMT salt is the saccharinate salt. In an embodiment, the 5-MeO-DMT salt is crystalline. In an embodiment, the crystalline 5-MeO-DMT saccharinate salt is characterised by one or more peaks in an XRPD diffractogram as detailed in the Table below:

### XRPD Peak Data for Saccharinate Pattern 1.

| | | | Rel. |
|---|---|---|---|
| Peak No. | Angle 2 θ | d Value | Intensity |
| 1 | 5.196° | 16.993 | 0.314 |
| 2 | 9.572° | 9.232 | 0.019 |
| 3 | 10.180° | 8.682 | 0.928 |
| 4 | 11.395° | 7.759 | 0.138 |
| 5 | 12.951° | 6.830 | 0.051 |
| 6 | 13.423° | 6.591 | 0.159 |
| 7 | 14.998° | 5.902 | 0.362 |
| 8 | 15.182° | 5.831 | 0.366 |
| 9 | 16.169° | 5.477 | 0.228 |
| 10 | 16.806° | 5.271 | 0.206 |
| 11 | 17.542° | 5.052 | 0.295 |
| 12 | 17.845° | 4.966 | 0.075 |
| 13 | 18.421° | 4.813 | 0.027 |
| 14 | 18.736° | 4.732 | 0.056 |
| 15 | 19.167° | 4.627 | 0.037 |
| 16 | 19.672° | 4.509 | 0.092 |
| 17 | 20.213° | 4.390 | 0.084 |
| 18 | 20.868° | 4.253 | 1.000 |
| 19 | 21.701° | 4.092 | 0.113 |
| 20 | 22.138° | 4.012 | 0.036 |
| 21 | 22.613° | 3.929 | 0.069 |
| 22 | 22.987° | 3.866 | 0.293 |
| 23 | 23.949° | 3.713 | 0.025 |
| 24 | 24.396° | 3.646 | 0.337 |
| 25 | 25.231° | 3.527 | 0.076 |
| 26 | 25.463° | 3.495 | 0.175 |
| 27 | 25.948° | 3.431 | 0.084 |
| 28 | 26.616° | 3.346 | 0.165 |
| 29 | 27.555° | 3.234 | 0.247 |
| 30 | 28.286° | 3.153 | 0.187 |
| 31 | 28.735° | 3.104 | 0.039 |
| 32 | 29.676° | 3.008 | 0.032 |
| 33 | 29.961° | 2.980 | 0.037 |
| 34 | 30.413° | 2.937 | 0.048 |
| 35 | 31.037° | 2.879 | 0.040 |
| 36 | 31.781° | 2.813 | 0.025 |

In an embodiment, the crystalline 5-MeO-DMT saccharinate salt is characterised by one or more of: One or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty one or more, twenty two or more, twenty three or more, twenty four or more, twenty five or more, twenty six or more, twenty seven or more, twenty eight or more, twenty nine or more, thirty or more, thirty one or more, thirty two or more, thirty three or more, thirty four or more, thirty five or more, or thirty six peaks in an XRPD diffractogram as detailed in the Table above; One or more, two or more, three or more, four or more or five or more peaks in an XRPD diffractogram with a relative intensity of over 0.1, 0.2, 0.3, 0.4, 0.5, 0.6, 0.7, 0.8 or 0.9 as detailed in the Table above; A weight loss of about 0.7% between 25-200° C., as measured by TGA thermogram; A weight loss of about 0.2-1.2% between 25-200° C., as measured by TGA thermogram; A weight loss of about 0.2, 0.3, 0.4, 0.5, 0.6, 0.7, 0.8, 0.9, 1.0, 1.1 or 1.2% between 25-200° C., as measured by TGA thermogram; A melting endothermic event with an onset of around 100.0° C. and an enthalpy of 76.6 J/g, as measured in a DSC thermogram; A melting endothermic event with an onset of around 95-105° C. and an enthalpy of around 70-80 J/g, as measured in a DSC thermogram; A melting endothermic event with an onset of around 95, 96, 97, 98, 99, 100, 101, 102, 103, 104 or 105° C. and an enthalpy of around 70, 71, 72, 73, 74, 75, 76, 77, 78, 79 or 80 J/g, as measured in a DSC thermogram; A vitrification around 27.1° C., as measured in a DSC thermogram with a cooling ramp of 10° C./min from 220° C. to −90° C.; A vitrification around 22-32° C., as measured in a DSC thermogram with a cooling ramp of 10° C./min from 220° C. to −90° C.; A vitrification around 22, 23, 24, 25, 26, 27, 28, 29, 30, 31 or 32° C., as measured in a DSC thermogram with a cooling ramp of 10° C./min from 220° C. to −90° C.; A glass transition around 33.8° C., as measured in a DSC thermogram with a cooling ramp of 10° C./min from 220° C. to −90° C.; and/or A glass transition around 28-38° C., as measured in a DSC thermogram with a cooling ramp of 10° C./min from 220° C. to −90° C.; and/or A glass transition around 28, 29, 30, 31, 32, 33, 34, 35, 36, 37 or 38° C., as measured in a DSC thermogram with a cooling ramp of 10° C./min from 220° C. to −90° C.

In an embodiment, the salt anion is an aryl carboxylate. In an embodiment, the aryl carboxylate is substituted with one to three R groups. In an embodiment the one or more R groups are independently selected from: alkynyl, carbonyl, aldehyde, haloformyl, alkyl, halide, hydroxy, alkoxy, carbonate ester, carboxylate, carboxyl, carboalkoxy, methoxy, hydroperoxy, peroxy, ether, hemiacetal, hemiketal, acetal, ketal, orthoester, methylenedioxy, orthocarbonate ester, carboxylic anhydride, carboxamide, secondary, tertiary or quaternary amine, primary or secondary ketimine, primary or secondary aldimine, imide, azide, azo, cyanate, isocyanate, nitrate, nitrile, isonitrile, nitrosooxy, nitro, nitroso, oxime, pyridyl, carbamate, sulfhydryl, sulphide, disulfide, sulfinyl, sulfonyl, sulfino, sulfo, thiocyanate, isothiocyanate, carbonothioyl, carbothioic S-acid, carbothioic O-acid, thiolester, thionoester, carbodithioic acid, carbodithio, phosphino, phosphono, phosphate, borono, boronate, borino or borinate. In an embodiment the one or more R groups are independently selected from: C₁-C₆ alkyl, C₁-C₆ alkoxy, C₁-C₆ alkenyl or C₁-C₆ alkynyl, and where each of these may be optionally substituted with one to three R groups as previously described.

In an embodiment, the 5-MeO-DMT is administered alongside one or more pharmaceutically acceptable carriers or excipients. The 5-MeO-DMT and the one or more pharmaceutically acceptable carriers or excipients together form a pharmaceutical composition. There is therefore provided a pharmaceutical composition comprising 5-MeO-DMT and one or more pharmaceutically acceptable carriers or excipients for use as a medicament, wherein the pharmaceutical composition is administered as set out herein. In an embodiment, the 5-MeO-DMT or the pharmaceutical composition comprising 5-MeO-DMT and one or more pharmaceutically acceptable carriers or excipients, is for use in a method of one or more of: treating mental disorders, in particular treatment resistant depression, major depressive disorder, persistent depressive disorder, alcohol use disorder, anxiety disorder, post-traumatic stress disorder (PTSD), body dysmorphic disorder, obsessive-compulsive disorder, eating disorder and psychoactive substance abuse.

In an embodiment, the pharmaceutical composition comprising 5-MeO-DMT and one or more pharmaceutically acceptable carriers or excipients comprises 0.0005 mg to 100 mg, 0.001 mg to 100 mg, 0.005 mg to 100 mg, 3 mg to 15 mg, 1 mg to 8 mg, 1 mg to 10 mg, 0.5 mg to 10 mg, 0.5 mg to 25 mg, 0.1 mg to 50 mg or 0.05 mg to 100 mg of 5-MeO-DMT. In an embodiment, the first or second dose of 5-MeO-DMT comprises 0.0005 mg to 100 mg, 0.001 mg to 100 mg, 0.005 mg to 100 mg, 3 mg to 15 mg, 1 mg to 8 mg, 1 mg to 10 mg, 0.5 mg to 10 mg, 0.5 mg to 25 mg, 0.1 mg to 50 mg or 0.05 mg to 100 mg of 5-MeO-DMT. The level of the active agent can be adjusted as required by need for example to suit a certain patient group (e.g. the elderly) or the conditions being treated.

In an embodiment, there is provided a method of treating one or more conditions comprising administration, as described herein, of 5-MeO-DMT to a subject in need thereof. In an embodiment, there is provided a method of treating depression comprising administration, as described herein, of 5-MeO-DMT to a subject in need thereof. In an embodiment, there is provided a method of treating treatment-resistant depression comprising administration, as described herein, of 5-MeO-DMT to a subject in need thereof. In an embodiment, there is provided a method of treating one or more of the conditions described herein, the method comprising administration, as described herein, of 5-MeO-DMT to a subject in need thereof.

In an embodiment, there is provided a pharmaceutical kit comprising a first applicator containing 5-MeO-DMT and a second applicator containing 5-MeO-DMT. In an embodiment, the dose of 5-MeO-DMT in the second applicator is higher than that in the first. In an embodiment, the applicators are single use. In an embodiment, the applicators are any of the well-known applicators for pharmaceutical compounds. In an embodiment, the applicators are intranasal. In an embodiment, the first applicator is a first type of applicator (e.g. intranasal) and the second applicator is a second type of applicator (e.g. orally disintegrating tablet). In an embodiment, the pharmaceutical kit further comprises instructions for use. In an embodiment, the pharmaceutical kit further comprises anti-tampering means. In an embodiment, the pharmaceutical kit further comprises anti-abuse means.

In an embodiment, the pharmaceutical kit comprises a first single use intranasal applicator comprising a dry powder pharmaceutical composition of 5-MeO-DMT and a second single use intranasal applicator comprising a dry powder pharmaceutical composition of 5-MeO-DMT. In an embodiment, the dose of 5-MeO-DMT in the second single use intranasal applicator is higher than that in the first applicator. In an embodiment, the dose of 5-MeO-DMT in the first or second applicator is as described herein. In an embodiment, there is provided a pharmaceutical kit comprising a first single use Aptar Unidose (UDS) Powder Nasal Spray System and a second single use Aptar Unidose (UDS) Powder Nasal Spray System. In an embodiment, the pharmaceutical kit as described herein is for use in a method of treatment of one or more conditions as described herein. In an embodiment, the pharmaceutical kit as described herein is for use in a method of treatment of one or more conditions as described herein according to a method of administration as described herein.

In an embodiment, there is provided a dose-proportional salt of 5-MeO-DMT for use in the methods disclosed herein. In an embodiment, the dose-proportional salt of 5-MeO-DMT is the benzoate salt. A double-blind, randomised, Phase 1, single ascending dose study to evaluate the safety, tolerability and pharmacokinetic profile of intranasal 5-MeO-DMT HCl (5-MeO-DMT HCl, HPMC, water for injection (WFI) and a sodium hydroxide solution to adjust pH) in healthy subjects was performed. The mean (+/−SD) 5-MeO-DMT plasma log concentration-time plot is shown in FIG. 1. It can be seen that 5-MeO-DMT HCl does not display dose-proportional pharmacokinetics, with the mean concentration profiles displayed for 5 mg, 8 mg, 10 mg, 11 mg and 14 mg all being substantially similar.

A double-blind, randomised, Phase 1, single ascending dose study to evaluate the safety, tolerability and pharmacokinetic profile of intranasal 5-MeO-DMT benzoate in healthy subjects was performed. The mean (+/−SD) 5-MeO-DMT plasma linear concentration-time plot and plasma log concentration-time plot are shown in FIGS. 2 and 3, respectively. The pharmacokinetics were shown to be approximately dose linear. No dose exceeded the maximum exposure limits defined by previous preclinical work in dogs: Cmax: 421 ng/mL or AUC 220 h·ng/mL. The mean (+/−SD) 5-MeO-DMT plasma linear concentration-time plot and plasma log concentration-time plot are shown in FIGS. 9 and 10, respectively. The mean Cmax was 29 ng/mL for the 12 mg dosage. The mean Tmax was 9.5 minutes whilst the mean half-life (T½) was 21 minutes. Bufotenin, the O-demethylated metabolite of 5-MeO-DMT, was only detected at very low levels at the 6 mg dose level after the 16 minutes time point.

In an embodiment, there is provided a psychedelic compound for use in the treatment of one or more conditions as described herein, according to a method as described herein. In an embodiment, there is provided a tryptamine alkaloid for use in the treatment of one or more conditions as described herein, according to a method as described herein. In an embodiment, there is provided psilocybin for use in the treatment of one or more conditions as described herein, according to a method as described herein. In an embodiment, there is provided psilocin for use in the treatment of one or more conditions as described herein, according to a method as described herein. In an embodiment, there is provided a salt of psilocin for use in the treatment of one or more conditions as described herein, according to a method as described herein. In an embodiment, there is provided psilocin benzoate for use in the treatment of one or more conditions as described herein, according to a method as described herein. In an embodiment, there is provided psilocin benzoate for use in the treatment of one or more conditions as described herein, according to a method as described herein, wherein the psilocin benzoate is administered intravenously.

In an embodiment, the 1^{st} dose of the compound administered according to the methods as described herein is equivalent to 5% of the dosage amount of the 2^{nd} dose. In an embodiment, it is 10%, 15%, 20%, 25%, 30%, 35%, 40%, 45%, 50%, 55%, 60%, 65%, 70%, 75%, 80%, 85%, 90%, 95% or 100%. In an embodiment, the methods described herein incorporate one or more further administrations of the compound. It will be understood that references to ‘5-MeO-DMT’ herein mean 5-MeO-DMT free base, or a pharmaceutically acceptable salt, prodrug, hydrate, ester, co-crystal or deuterated form thereof, or a pharmaceutical composition comprising the aforementioned.

In an embodiment, the patient to be treated has at least moderate major depressive disorder (MDD), (single or recurrent episode as informed by Diagnostic and Statistical Manual of Mental Disorders, Fifth Edition [DSM-5] criteria; if single episode, duration of ≥3 months and ≤2 years) based on medical records, clinical assessment, and documented completion of the version 7.0.2 Mini International Neuropsychiatric Interview (MINI). In an embodiment, the patient is diagnosed with TRD defined as failure to respond to an adequate dose and duration of at least 2 pharmacological treatments, in the current episode, based on the MGH ATRQ assessment. In an embodiment, augmentation with an add-on treatment counts as a second treatment. In an embodiment, the patient has not failed more than 5 prior pharmacological treatments in the current episode. In an embodiment, psychotherapy is not counted towards treatment failure. In an embodiment, the patient has a Hamilton Depression Rating Scale (HDRS) (17 item) score ≥19 at baseline/prior to treatment. In an embodiment, the patient has a CGI-S ≥4 at baseline/prior to treatment. In an embodiment, the patient does not have a current or past history of schizophrenia, post-traumatic stress disorder, psychotic disorder including psychotic depression, bipolar disorder, delusional disorder, schizoaffective disorder, or any other severe psychiatric disorder. In an embodiment, the patient does not have a current or past history of schizophrenia, post-traumatic stress disorder, psychotic disorder including psychotic depression, bipolar disorder, delusional disorder, schizoaffective disorder, or any other severe psychiatric disorder as assessed by medical history and a structured clinical interview (MINI). In an embodiment, the patient does not have a current personality disorders: Cluster A (paranoid, schizoid, schizotypal), Cluster B (antisocial, borderline, histrionic, narcissistic), or Cluster C (avoidant, dependent, -obsessive compulsive). In an embodiment, the patient does not have a current personality disorders: Cluster A (paranoid, schizoid, schizotypal), Cluster B (antisocial, borderline, histrionic, narcissistic), or Cluster C (avoidant, dependent, -obsessive compulsive) assessed via McLean Screening Instrument for Borderline Personality Disorder (MSI-BPD), MINI, and clinical judgement. In an embodiment, the patient does not have a first-degree family history of schizophrenia, bipolar disorder, delusional disorder, or schizoaffective disorder. In an embodiment, the patient does not have current (within the last year) alcohol or substance use disorder (other than caffeine or nicotine). In an embodiment, the patient does not have current (within the last year) alcohol or substance use disorder (other than caffeine or nicotine) according to DSM-5 and assessed by the MINI. In an embodiment, the patient has not at any time been unresponsive to ketamine or esketamine. In an embodiment, the patient has not at any time been unresponsive to an adequate course of treatment with electroconvulsive therapy (ECT). In an embodiment, the patient has not at any time been unresponsive to an adequate course of treatment with electroconvulsive therapy (ECT) defined as at least 7 treatments with unilateral/bilateral ECT, or has received vagal nerve stimulation or has received deep brain stimulation. In an embodiment, the patient has not had suicidal ideation with some intent to act within 12 months prior to treatment. In an embodiment, the patient has not had suicidal ideation with some intent to act within 12 months prior to treatment, based on the C-SSRS, corresponding to a response of “Yes” on Item 4 (active suicidal ideation with some intent to act, without specific plan) or Item 5 (active suicidal ideation with specific plan and intent) for suicidal ideation on the C-SSRS, or any suicidal behavior prior to treatment. In an embodiment, the patient has not attempted suicide and/or any other self-injurious behaviour within 12 months prior to treatment. In an embodiment, the patient does not have a history of any medical condition that could make receiving a sympathomimetic drug harmful because of transient and marked increases in blood pressure and heart rate. In an embodiment, the patient does not have atherosclerotic cardiovascular disease, obstructive coronary artery disease, myocardial infarction, hospitalisation for unstable angina, stroke, hospitalisation for transient ischemic attacks, known aortic or cerebral aneurysm or revascularization procedure within 12 months prior to treatment. In an embodiment, the patient does not have significant valvular heart disease, history of hospitalisation for heart failure and/or history of hospitalisation for atrial or ventricular arrhythmias. In an embodiment, the patient does not have a history of uncontrolled hypertension despite antihypertensive therapy and/or any past history of a hospital admission for hypertension. In an embodiment, the patient does not have controlled hypertension on antihypertensive therapy with repeated clinic seated or semi-recumbent systolic blood pressure ≥130 mmHg or diastolic blood pressure ≥80 mmHg. In an embodiment, the patient does not have repeated clinic seated or semi-recumbent systolic blood pressure ≥140 mmHg or diastolic blood pressure ≥90 mmHg. In an embodiment, the patient does not have one or more of hypo/hyperthyroidism with abnormal thyroid stimulating hormone values, uncontrolled or insulin dependent diabetes with HbA1c >8, renal failure with Creatinine Clearance <45 mL/min. In an embodiment, the patient does not have any seizure disorder and/or any seizure within 2 years of treatment. In an embodiment, the patient does not have any clinically significant results on ECG or a QT interval corrected using Fridericia's formula (QTcF) ≥450 msec for males or ≥470 msec for females prior to treatment. In an embodiment, the patient does not have any history of intolerance to 5-MeO-DMT or related compounds. In an embodiment, the patient does not have any nasal obstruction, blockage, or symptoms of congestion at the time of treatment. In an embodiment, the patient is not a female patient who is pregnant, lactating, or of childbearing potential who is not willing or able to use adequate forms of contraception during treatment. In an embodiment, the patient is not a male patient who is not willing or able to use adequate forms of contraception during treatment. In an embodiment, the patient does not have a personal or family history of malignant hyperthermia. In an embodiment, the patient has discontinued one or more of the following at least 5 half-lives prior to treatment: Medications that antagonise the serotonin 2A receptor, Medications with serotonergic activity (e.g., SSRIs, SNRIs, efavirenz, lithium), Tramadol, Opioids, Antiviral Medications, St. John's Wort, Medications that inhibit UGT1A9 or UGT1A10 enzymes, Monoamine Oxidase Inhibitors (MAOIs) and/or Medications that inhibit aldehyde or alcohol dehydrogenase.

In an embodiment, there is provided a method of rapidly treating one or more of cognitive dysfunction, negative thinking, bipolar disorder, postnatal depression/major depressive disorder, postpartum depression/major depressive disorder, social/emotional withdrawal of detachment, psychomotor retardation, anxiety and/or sleep disturbance.

In an embodiment, there is provided a method of rapidly treating a sleep disturbance, such as insomnia, hypersomnia, circadian rhythm disorder, parasomnia, sleep-related breathing disorder, sleep-related movement disorder, and/or an idiopathic sleep disturbance. In an embodiment, the patient is suffering from: one or more mental or nervous system disorders associated with the sleep disturbance, or a treatment resistant form thereof, selected from one or more of a disorder characterised by depressive episodes associated with the sleep disturbance, major depressive disorder (MDD) associated with the sleep disturbance, postpartum depression (PPD) associated with the sleep disturbance, compromised maternal functioning, compromised maternal functioning wherein the patient has a Barkin Index of Maternal Functioning (BIMF) score of 80 or below, such as 65 or below, compromised maternal functioning wherein the treatment improves maternal functioning, compromised maternal functioning wherein the treatment improves maternal functioning reflected by an improvement of the BIMF total score by 10% or more, preferably by 20% or more, compromised maternal functioning wherein the treatment improves maternal functioning reflected by at least an improvement in the BIMF total score on day 7; on day 14; and/or on day 28 after the last administration of 5-MeO-DMT, by at least an improvement in the BIMF total score on day 1, 2, 3, 4, 5, 6 and/or 7; on day 14; and/or on day 28 after the last administration of 5-MeO-DMT, bipolar disorder associated with the sleep disturbance, bipolar II disorder associated with the sleep disturbance, bipolar I disorder associated with the sleep disturbance, a current major depressive episode, seasonal affective disorder associated with the sleep disturbance, persistent depressive disorder associated with the sleep disturbance, anxiety disorder associated with the sleep disturbance, separation anxiety disorder associated with the sleep disturbance, agoraphobia associated with the sleep disturbance, generalised anxiety disorder (GAD) associated with the sleep disturbance, social anxiety disorder (SAD) associated with the sleep disturbance, panic disorder associated with the sleep disturbance, phobia associated with the sleep disturbance, substance/medication induced anxiety disorder associated with the sleep disturbance, somatic symptom disorder associated with the sleep disturbance, an obsessive compulsive or related disorder associated with the sleep disturbance, body dysmorphic disorder (BDD) associated with the sleep disturbance, post-traumatic stress disorder (PTSD) associated with the sleep disturbance, a pain disorder associated with the sleep disturbance, chronic pain associated with the sleep disturbance, fibromyalgia associated with the sleep disturbance, migraine associated with the sleep disturbance, a mental and behavioural disorder due to psychoactive substance use associated with the sleep disturbance, substance use disorder (SUD) associated with the sleep disturbance, a psychotic disorder associated with the sleep disturbance, schizophrenia associated with the sleep disturbance, Huntington's disease associated with the sleep disturbance, Parkinson's disease associated with the sleep disturbance, dementia associated with the sleep disturbance, Alzheimer's dementia associated with the sleep disturbance, Parkinson's disease dementia associated with the sleep disturbance, dementia with Lewy Bodies associated with the sleep disturbance, vascular dementia associated with the sleep disturbance, fronto-temporal dementia associated with the sleep disturbance, eating disorder associated with the sleep disturbance, an eating disorder suffers from a treatment resistant form of the disorder, attention deficit hyperactivity disorder (ADHD) associated with the sleep disturbance, a personality disorder associated with the sleep disturbance, schizotypal personality disorder associated with the sleep disturbance, a borderline personality disorder (BPD) associated with the sleep disturbance, an autism spectrum disorder (ASD) associated with the sleep disturbance and/or chronic fatigue syndrome associated with the sleep disturbance.

In an embodiment, there is provided a method of rapidly treating bipolar disorder in a patient in need thereof. In an embodiment, the bipolar disorder is bipolar I or bipolar II. In an embodiment, the patient: suffers from a current major depressive episode, the patient has a Montgomery-Asberg Depression Rating Scale (MADRS) total score of equal to or greater than 19, such as equal to or greater than 24, in particular equal to or greater than 37, the patient has a Bipolar Depression Rating Scale (BDRS) total score of equal to or greater than 19, such as equal to or greater than 24, in particular equal to or greater than 37, the patient had no adequate improvement after at least two adequate courses of therapy, the patient had no adequate improvement after at least two adequate courses of therapy, wherein at least one of the two courses was a pharmacotherapy, the patient had no adequate improvement after at least two adequate courses of pharmacotherapy, the patient has a Young Mania Rating Scale (YMRS) total score less than or equal to 8 and/or the 5-MeO-DMT or salt thereof is administered at a dose or in a dosage regimen that causes the patient to experience a complete mystical experience.

In an embodiment, there is provided a method of rapidly treating anxiety in a patient in need thereof. In an embodiment, the patient: is suffering from is a subthreshold anxiety, has a comorbidity of anxiety and a further diagnosed disorder, is also suffering from a mental or nervous system disorder, is also suffering from a mental or nervous system disorder suffers from a treatment resistant form of the disorder, is also suffering from a disorder characterised by depressive episodes, is also suffering from major depressive disorder (MDD), is also suffering from postpartum depression (PPD), suffers in addition from compromised maternal functioning, has a Barkin Index of Maternal Functioning (BIMF) score of 80 or below, such as 65 or below. In an embodiment, the patient is suffering from a treatment resistant form of one or more of the above conditions or disorders.

In an embodiment, there is provided a method of rapidly treating one or more as disorders characterised by depressive episodes for example, Major Depressive Disorder (MDD), Postpartum Depression (PPD), Persistent Depressive Disorder, Seasonal Affective Disorder and Bipolar Disorder (BD), such as Bipolar I Dis-order and Bipolar II Disorder; Anxiety Disorder, for example Separation Anxiety Disorder, Agoraphobia, Generalised Anxiety Disorder (GAD), Social Anxiety Disorder (SAD), Panic Disorder, Phobias, and Substance/Medication Induced Anxiety Disorder; Somatic Symptom Disorder; Obsessive Compulsive and Related Disorders, for example, Obsessive Compulsive Disorder (OCD) and Body Dysmorphic Disorder (BDD); Post-Traumatic Stress Disorder (PTSD); Pain Disorders, for example Chronic Pain, Fibromyalgia and Migraine; Mental and Behavioural Disorders due to Psychoactive Substance Use, for example, Substance Use Disorder (SUD); Psychotic Disorders, for example Schizophrenia; Parkinson's Disease; Dementia, for example Alzheimer's Dementia (AD), Parkinson's Disease Dementia (PDD), Dementia with Lewy Bodies, Vascular Dementia, Fronto-Temporal Dementias; Eating Disorders; Attention Deficit Hyperactivity Disorder (ADHD); Personality Disorders, for example Schizotypal Personality Disorder and Borderline Personality Disorder; Autism Spectrum Disorder; Chronic Fatigue Syndrome; or a medical health condition leading to an associated mental or nervous system condition, for example anxiety due to Traumatic Brain Injury (TBI), HIV infection, or post COVID condition.

Treatment as indicated herein with 5-MeO-DMT or a pharmaceutically acceptable salt, prodrug, hydrate, ester, co-crystal or deuterated form thereof (or a pharmaceutical composition comprising said compound or salt) leads to a clinical response. The response may be assessed by at least a score of “much improved” in the Clinical Global Impression-Improvement (CGI-I) score or the Patient Global Impression-Improvement (PGI-I) score, which improvement preferably occurs not later than about 2 hours after the administration of 5-MeO-DMT, or a pharmaceutically acceptable salt, prodrug, hydrate, ester, co-crystal or deuterated form thereof.

The clinical response, as assessed by at least a score of “much improved” in the CGI-I score or the PGI-I score, preferably persists until at least 1, 2, 3, 4, 5, 6, 7, 8, 9, 10, 11, 12, 13, 14, 15, 16, 17, 18, 19 or 20 weeks after the administration of 5-MeO-DMT, or a pharmaceutically acceptable salt, prodrug, hydrate, ester, co-crystal or deuterated form thereof.

The clinical response may also be assessed by improvement of the MADRS or HAM-D score, compared to the respective score prior to the administration of 5-MeO-DMT. The clinical response may be assessed by at least 50% improvement of the MADRS or HAM-D score, compared to the respective score prior to the administration of 5-MeO-DMT.

This response preferably occurs not later than about 2 hours after the administration of 5-MeO-DMT or a pharmaceutically acceptable salt, prodrug, hydrate, ester, co-crystal or deuterated form thereof. Further, a remission of depressive symptoms, as assessed by a MADRS score equal to or less than 10, or a HAM-D score equal to or less than 7, preferably occurs not later than about 2 hours after the administration of 5-MeO-DMT or a pharmaceutically acceptable salt, prodrug, hydrate, ester, co-crystal or deuterated form thereof. The clinical response, as may be assessed by at least 50% improvement of the MADRS or HAM-D score, compared to the respective score prior to treatment, preferably persists until at least 1, 2, 3, 4, 5, 6, 7, 8, 9, 10, 11 or 12 weeks after the administration of 5-MeO-DMT, or a pharmaceutically acceptable salt, prodrug, hydrate, ester, co-crystal or deuterated form thereof.

In an embodiment, the treatment response is seen within the herein disclosed periods of time following the single biphasic administration of 5-MeO-DMT, or a pharmaceutically acceptable salt, prodrug, hydrate, ester, co-crystal or deuterated form thereof, or a pharmaceutical composition thereof. The person skilled in the art will appreciate that this may refer to a single “treatment” comprising an initial administration of 5-MeO-DMT followed by a subsequent administration of a higher dose of 5-MeO-DMT, as disclosed herein, wherein both administrations take place on the same day, as disclosed herein.

As used in the context of the present invention, a “patient” to be treated is a human subject who is diagnosed as suffering from one or more of the diseases/conditions/disorders, as disclosed herein, by a licensed professional in accordance with accepted medical practice.

Diagnosis of one or more of the diseases/conditions/disorders, as disclosed herein, optionally a mental disorder or a nervous system disorder can, for instance, be in accordance with the Diagnostic and Statistical Manual of Mental Disorders-Fifth Edition (DSM-5) published by the American Psychiatric Association. In some instances, as is apparent from the discussion of specific conditions below, the criteria may be modified or supplemented to better define patients or patient groups particularly benefiting from a treatment according to the invention. The diagnosis will in any event be by a physician or a psychologist. It is not sufficient that the human subject himself/herself considers that he/she is suffering from the disorder.

As used in the context of the present invention, unless otherwise noted, the terms “treating” and “treatment” shall include the management and care of a patient for the purpose of combating a disease, condition, or disorder and includes the administration of compounds and methods according to the present invention to alleviate the signs and/or symptoms of the disease or eliminate the disease, condition, or disorder. “Treatment of one or more of the diseases/conditions/disorders, as disclosed herein” shall include the management and care of a patient for the purpose of combating negative thinking and includes the administration of compounds and methods according to the present invention to alleviate the signs and/or symptoms of the one or more of the diseases/conditions/disorders, as disclosed herein.

The patient may suffer from treatment resistant disease. Treatment resistance means that the patient had no adequate improvement after at least two adequate courses of therapy. The patient in particular had no adequate improvement after at least two adequate courses of therapy, wherein at least one of the two courses was a pharmacotherapy; for instance, the patient had no adequate improvement after at least two adequate courses of pharmacotherapy. The at least two prior courses of treatment were in particular administered in the current episode of the disease, for instance, if the patient suffers from a disorder characterised by depressive episodes, in the current episode of depression.

As used in the context of the present invention, unless otherwise noted, the term “therapeutically effective amount” shall mean the amount of active compound or pharmaceutical ingredient that elicits the biological or clinical response in a human that is being sought by a researcher, medical doctor or other clinician, which includes alleviation of the signs and/or symptoms of the one or more of the diseases/conditions/disorders, as disclosed herein.

As used herein, “Clinical response” and/or “clinically significant reduction” and/or “clinically significant response” includes, but is not limited to, improvements on rating scales such as the Clinical Global Impression-Severity scale (CGI-S), the Patient Global Impression-Severity scale (PGI-S), the Clinical Global Impression-Improvement scale (CGI-I) or the Patient Global Impression-Improvement scale (PGI-I) and further includes, but is not limited to, endpoints such as the Montgomery-Asberg depression/major depressive disorder Rating Scale (MADRS) or the 17-item Hamilton depression/major depressive disorder Rating Scale (HAM-D) for depression/major depressive disorder and persistent depressive disorder, anxiety symptoms e.g. as measured by the Beck Anxiety Inventory (BAI), the Hamilton Anxiety Scale (HAM-A) or the State-Trait Anxiety Inventory (STAI) for anxiety disorder, the Clinician-Administered Posttraumatic Stress Disorder Scale (CAPS) for posttraumatic stress disorder, the Yale-Brown Obsessive Compulsive Scale Modified for Body Dysmorphic Disorder (BDD-YBOCS) for body dysmorphic disorder, the Yale-Brown Obsessive Compulsive Scale (Y-BOCS) for obsessive-compulsive disorder, weight gain for anorexia nervosa, frequency of binge-purge episodes for bulimia nervosa, frequency of binge episodes for binge eating disorder, duration of abstinence or reduced substance use in psychoactive substance abuse and suicidality rating scales such as the Columbia-Suicide Severity Rating Scale (C-SSRS) or the suicidal thoughts item of the MADRS for suicidal ideation or the Clinical Global Impression-Severity of Suicidality-Revised (CGI-SS-R) scale (the CGI-SS-R is derived from the CGI-S, and is scored 0=Normal, Not At All Suicidal; 1=Questionably Suicidal; 2=Mildly Suicidal; 3=Moderately Suicidal; 4=Markedly Suicidal; 5=Severely Suicidal; 6=Extremely Suicidal). When assessing a clinical response at an early time point after drug administration (e.g. at 2 hours) based on endpoints which have been developed for a longer recall period, a rational modification of such endpoint (e.g. changing the MADRS recall period to 2 hours and carrying forward the sleep item recorded at baseline before drug administration) may be applied.

The severity of a condition as well as changes of the severity can be assessed by the Clinical Global Impression (CGI) rating scales which are measures of symptom severity, treatment response and the efficacy of treatments.

The CGI rating scales were developed to provide a brief, stand-alone assessment of the clinician's view of the patient's global functioning prior to and after a treatment (Busner, J. and Tagrum, S. D., 2007. The Clinical Global Impressions Scale: Applying a Research Tool in Clinical Practice. Psychiatry 2007, 29-37).

The CGI-Severity (CGI-S) is based on one question the clinician has to answer: “Considering your total clinical experience with this particular population, how mentally ill is the patient at this time?” This is rated on the following seven-point scale: 1=normal, not at all ill; 2=borderline mentally ill; 3=mildly ill; 4=moderately ill; 5=markedly ill; 6=severely ill; 7=among the most extremely ill patients.

The CGI-S can be used to assess treatment success by comparing scores before and after treatment.

A clinical response may be reflected by a reduction in the Clinical Global Impression-Severity (CGI-S) score. According to the invention, a reduction in the CGI-S score means that the CGI-S is reduced by at least 1. Preferably, the CGI-S is reduced by at least 2 and/or to a score of 0. It is especially preferred if the CGI-S is reduced by at least 3 and/or to a score of 0.

Alternatively, treatment success can be assessed using the CGI-Improvement (CGI-I), which is similarly simple in its format. After the treatment, the clinician compares the patient's overall clinical condition to the one prior to the treatment (the so-called baseline value). Again, only one query is rated on a seven-point scale: “Compared to the patient's condition at admission to the project [prior to medication initiation], this patient's condition is: 1=very much improved since the initiation of treatment; 2=much improved; 3=minimally improved; 4=no change from baseline (the initiation of treatment); 5=minimally worse; 6=much worse; 7=very much worse since the initiation of treatment.”

The Patient Global Impression scale (PGI), also known as Subject Global Impression (SGI), is the counterpart to the Clinical Global Impressions scale (CGI). It consists of one item based on the CGI and adapted to the patient. It can measure disease severity (PGI-S) or disease improvement (PGI-I).

Individual items of scales as described herein as well as sub-combinations of individual items may be used to assess specific disease aspects.

Numerous scales have been suggested to assess severity of one or more conditions or disorders, such as one or more conditions or disorders, such as one or more conditions or disorders, such as a mental disorder or a nervous system disorder. Such scales are based on tests which may be self-administered or administered by a clinician/physician. Scales which may be used according to the invention include those known in the art for diagnosis and/or monitoring the one or more conditions or disorders, such as a mental disorder or a nervous system disorder are discussed in more detail herein. Treatment outcome is assessed by using one or more indices or scales at one or more time points after completion of a treatment course.

The assessment may be carried out after the complete mystical experience has subsided. An appropriate point in time for an early assessment is generally about 2 to 3 hours after the last administration. An early assessment may generally be carried out, for instance, about 2 hours or about 3 hours after the last administration. An assessment of an effect on, for example, sleep disturbance can, however, be carried out at the earliest on the day after the treatment (i.e., on day 1) so that the treated patient/subject had the opportunity to sleep for at least one night.

Thus, an assessment at day 1 or on day 1 means an assessment on the day following the administration. The assessment may be carried out not earlier than 12 hours after the last administration and in any event optionally not earlier than one night after the last administration and not later than 36 hours after the last administration. The assessment may be carried out after about 24 hours. An assessment at day 7 or on day 7 means an assessment on the seventh day following the administration (the day of administration is day 0). Analogous definitions apply for other assessment timings measured in days.

When assessing a clinical response, for instance, using one of the scales to assess severity of one or more conditions or disorders, such as a mental disorder or a nervous system disorder, at an early time point after drug administration (e.g. at 1, 2 or 3 hours) based on endpoints which have been developed for a longer recall period (e.g. normally 7 days for the MADRS), a rational modification of such endpoint (e.g. changing the MADRS recall period to 1, 2 or 3 hours and carrying forward the sleep item recorded at baseline before drug administration) may be applied. The same applies with respect to any other scale applied herein, unless a recall period is specifically indicated. The considerations outlined apply for early time points because, on the one hand, in order to assess a clinical response, the influence of the patient's status before the treatment on any score recorded after treatment should be kept as low as possible, whereas on the other hand the sleep item cannot be assessed 1, 2 or 3 hours after drug administration. At later time points, for instance, on day 1 or later, typically all items of the relevant scales to assess a clinical response may be assessed, using, if necessary, an adapted recall period, so that it is not necessary to carry forward any pre-treatment score.

There are two fundamental types of sleep: rapid eye movement (REM) sleep and non-REM sleep. Non-REM sleep may be divided into four stages (1-IV). These non-REM stages correspond to an increasing depth of sleep. Non-REM and REM sleep alternate during each of the four to five cycles of normal human sleep each night. During the earlier proportion of the night, non-REM sleep is deeper and occupies a disproportionately large amount of time, particularly within the first cycle of sleep. As the night progresses, non-REM sleep becomes shallow and more of each cycle is allocated to REM sleep.

Normal healthy sleep consists of different phases as outlined above that proceed in successive, tightly regulated order through the night. Disruption of this tight regulation results in sleep disturbances. Sleep disturbance refers to conditions, whether idiopathic or occurring in the context of a medical condition such as for example one or more conditions or disorders, such as one or more conditions or disorders, such as a mental disorder or a nervous system disorder, that affect sleep quality, timing, or duration. It impacts a person's ability to properly function while the person is awake.

Common forms of sleep disturbances encompass disorders of initiating and maintaining sleep (insomnia), disorders of excessive somnolence (hypersomnia), disorders of sleep wake schedule (circadian rhythm disorders), dysfunctions associated with sleep, sleep stages, or partial arousals (parasomnia), disorders characterised by respiratory disturbance during sleep (sleep-related breathing disorders) and disorders characterised by abnormal movements during sleep (sleep-related movement disorders). Insomnia is a sleep disturbance where people have difficulty falling or staying asleep. People with insomnia have difficulty falling asleep; wake up often during the night and have trouble going back to sleep; wake up too early in the morning; have unrefreshing sleep; and/or have at least one daytime problem such as fatigue, sleepiness, problems with mood, concentration, accidents at work or while driving, etc. due to poor sleep.

Hypersomnia is characterised by excessive daytime sleepiness, and/or prolonged nighttime sleep. Sleep drunkenness is also a symptom found in hypersomnia patients/subjects. It is a difficulty transitioning from sleep to wake. Individuals experiencing sleep drunkenness report waking with confusion, disorientation, slowness and repeated returns to sleep.

Circadian rhythm disorders are characterised by chronic or recurring sleep disturbances due to alterations of the individual's internal circadian rhythm or due to misalignments between their circadian rhythm and their desired or required work or social schedule. This dyssynchrony may be transient or persistent. The ensuing clinical picture combines elements of both insomnia and hypersomnia. Sleep periods are usually shortened and disrupted, performance during the desired waking state is impaired, and temporary opportunities to revert to a regular sleep schedule are unsuccessful.

Parasomnia designates various forms of sleep disturbance characterised by abnormal behavioural or physiological activity (such as sleepwalking or nightmares) that people experience prior to falling asleep, while asleep, or during the arousal period between sleep and wakefulness. There are considerable variations in terms of characteristics, severity, and frequency. Parasomnia may compromise the quality of sleep.

Sleep-related breathing disorders are characterised by abnormal and difficult respiration during sleep. Respiration is a complex process that relies heavily on the coordinated action of the muscles of respiration and the (control centre in the) brain. One form of a sleep-related breathing disorder is central sleep apnoea. It occurs when the brain stops sending signals that control breathing, for instance, based on an underlying health condition. Central sleep apnoea has a potentially serious impact on sleep and the balance of oxygen and carbon dioxide in the blood. The reduction of airflow leads to intermittent hypoxia which in leads to sleep fragmentation due to microarousals or awakenings. A consequence may be excessive daytime sleepiness.

In sleep-related movement disorders repetitive, relatively simple, usually stereotyped, movements interfere with sleep or its onset. The most common of these are restless leg syndrome (RLS) and periodic limb movement disorder (PLMD). Not getting the proper amount or quality of sleep may lead to personality changes and may not only exacerbate existing mental illness, but also be a trigger for the development of mental illness. Sleep disturbance may also interfere with cognitive function and lead to memory impairment. A patient/subject who is deprived of sleep may experience difficulty making decisions, irritability, have problems with performance, and may have slower reaction times. Sleep loss may also adversely affect life by contributing to the development of obesity, diabetes, and heart disease.

Treatment of sleep disorders varies depending on the type and underlying cause. Maintenance of good sleep hygiene, a healthy sleep environment, and a consistent sleep-wake schedule are often considered as first-line treatment. If not successful, treatment also involves pharmacotherapy or psychotherapy. Available treatments are not successful in all patients/subjects, may be associated with side effects and/or require treatment over a long period of time to achieve a relevant treatment effect. In patients/subjects suffering from sleep disturbance in association with one or more conditions or disorders, such as a mental disorder or a nervous system disorder known treatments of the mental or nervous system disorder do not necessarily improve the sleep disturbance.

For instance, sleep disturbance is frequently associated with mental disorders, such as depression. However, treatment of depression does not necessarily lead to an improvement of the concomitant sleep disturbances. While most antidepressants have been proven to influence the sleep architecture, some classes of antidepressants improve sleep, but others may cause sleep impairment. Sleep may be assessed by measuring parameters such as sleep duration, sleep architecture, sleep latency, and the frequency and duration of awakenings throughout the night. The quantitative metrics may be measured using objective methods, including polysomnography, actigraphy, and the determination of sleep latency, or by way of self reported measures (questionnaires).

Polysomnography is a technique requiring that a patient/subject is monitored overnight at a specialised clinic. A variety of functions are measured throughout the night, including eye movements, brain and muscle activity, respiratory effort and airflow, blood oxygen levels, body positioning and movements, snoring, and heart rate. Another quantitative measurement is actigraphy. An actimetry sensor is worn to measure motor activity, which is recorded continually and used to assess sleep-wake cycles. This technique allows the patient/subject to continue normal routines while the required data are being recorded in a natural sleep environment.

Sleep latency may be measured by the multiple sleep latency test (MSLT). This test provides an objective measure to determine how long it takes a person to fall asleep across a multiplicity of test naps. An average sleep latency of approximately 10 minutes is considered to be normal; less than eight minutes is indicative of sleep disturbance (excessive daytime sleepiness). Accompanying analysis of brain activity may assist in the further diagnosis of the sleep disturbance.

Sleep-rating questionnaires capture ratings of components of sleep quality, such as perceptions of sleep depth, rousing difficulties, and restfulness after sleep, in addition to other factors that could affect sleep quality, such as comorbid conditions and medication use. The evaluation of the qualitative aspects of sleep experience is important, as sleep complaints may often persist despite normal values for quantitative measures of sleep. Questionnaires not only facilitate a quick and accurate assessment of a complex clinical problem, but they are potentially also helpful for tracking a patient's progress.

Various sleep quality indexes are known. The following indexes include examples of questionnaires to assess sleep in general and questionnaires to assess in particular insomnia, hypersomnia, circadian rhythm disorders and parasomnia, respectively. The invention is, however, not limited to the use of a particular index or questionnaire.

Some questionnaires rely on recall periods (recall windows) of several days or even weeks. While this may be appropriate for diagnosing sleep disturbances, it is not always appropriate for assessing treatment effects, in particular rapid onset of effect after treatment. For several of the questionnaires the recall period may be modified so that the scores obtained reflect a period after treatment. Questionnaires specifically discussed herein to assess effects of a treatment on sleep in patients/subjects suffering from specific conditions rely on a recall period that does not start earlier than the time point when complete mystical experiences have subsided after the last administration. To meet this criterion, the normally applied recall period is modified if necessary.

Sleep quality in general may be assessed, for instance, with the Sleep-50 questionnaire. The SLEEP-50 questionnaire consists of 50 items designed to screen for a variety of sleep disorders in the general population. The scale consists of nine subscales, reflecting some of the most common disorders and complaints related to sleep and the factors required for diagnosis such as sleep apnoea, insomnia, narcolepsy, restless legs/periodic leg movement disorder, circadian rhythm sleep disorder, sleepwalking, nightmares, factors influencing sleep, and the impact of sleep complaints on daily functioning. For each item, respondents are provided with a scale ranging from 1 (“not at all”) to 4 (“very much”) and are asked to indicate the extent to which the statement has matched their experience over the previous month or another appropriate recall window.

For diagnosing a sleep disorder not only the specific subscale (e.g., insomnia) must exceed a certain cut-off point, but respondents must also meet a cut-off of at least 3 or 4 (“rather much” or “very much”, respectively) on the subscale evaluating the impact of sleep complaints on daily functioning. Treatment success may be indicated (i) by a decrease of the score, preferably (ii) by a decrease to below the cut-off value. A common questionnaire assessing sleep disturbance is the Pittsburgh Sleep Quality Index. Other instruments are the insomnia severity index, the Espie sleep disturbance questionnaire and the patient/subject Reported Outcomes Measurement Information System (PROMIS®) Sleep Disturbance.

The Pittsburgh Sleep Quality Index (PSQI) assesses overall sleep quality and disturbances. The PSQI is a self-rated questionnaire comprising 19 questions. Respondents are asked to indicate how frequently they have experienced certain sleep difficulties over the past month or another appropriate recall window. The 19 self-rated questions assess a wide variety of factors relating to sleep quality, including estimates of sleep duration and latency and of the frequency and severity of specific sleep-related problems. These 19 items are grouped into seven component scores: (1) patient/subjective sleep quality; (2) sleep latency; (3) sleep duration; (4) habitual sleep efficiency; (5) sleep disturbances; (6) use of sleeping medication; (7) daytime dysfunction. Each component is assigned a score of 0 to 3. Higher scores indicate more acute sleep disturbances.

The seven component scores are then summed to yield one global score, with a range of 0-21 points, “0” indicating no difficulty and “21” indicating severe difficulties in all areas. A global score cut-off of 5 distinguishes poor from good sleepers. A global score >5 indicates that a patient/subject is having severe difficulties in at least two areas, or moderate difficulties in more than three areas. If treatment outcome is assessed using the PSQI, treatment success may be indicated (i) by a decrease of the score, preferably (ii) by a decrease to 5 or below.

The insomnia severity index (ISI) is a short questionnaire relating to patient/subjective sleep quality, severity of symptoms, patient/subjective satisfaction with sleep, the degree to which insomnia interferes with daily functioning, how noticeable the respondent feels his or her insomnia is compared to others, and the overall level of distress created by the sleep problem. Individual responses may be scored from 0 (=none) to 4 (=very); a higher total score corresponds to more severe insomnia. A total score of 0-7 indicates “no clinically significant insomnia,” 8-14 means “subthreshold insomnia,” 15-21 is “clinical insomnia (moderate severity),” and 22-28 means “clinical insomnia (severe)”. The recall window is two weeks. Another appropriate recall window may also be used. Treatment success may be indicated (i) by a decrease of the score, for instance, by >7 points, in particular >8 point; preferably (ii) by a decrease to below the cut-off value for clinically significant insomnia.

The Espie sleep disturbance questionnaire (SDQ) evaluates patient/subjective experiences of insomnia. With ratings on restlessness/agitation, mental overactivity, consequences of insomnia, and lack of sleep readiness, the SDQ is concerned specifically with beliefs about the sources of sleep issues. Respondents use a five-point scale to indicate how often certain statements about insomnia are representative of their experience. 1 means “never true,” while 5 means “very often true.” Higher scores are indicative of more dysfunctional beliefs about the causes and correlates of insomnia.

Treatment success may be indicated by a decrease of the score. The Patient-Reported Outcomes Information System (PROMIS)® Sleep Disturbance instrument is a universal measure to evaluate sleep disturbances. The instrument is available as a long form and 4 different short forms (e.g., 4-, 6-, and 8-items) and assesses self-reported perceptions of sleep quality, sleep depth, and any perceived difficulties related to getting and staying asleep over a 7-day period. Each item on the measure is rated on a 5-point scale. The raw scores on the items are summed to obtain a total raw score. Total raw scores are then converted into a standardised T-score using conversion tables. Treatment success may be indicated by a decrease of the T-score.

Hypersomnia or hypersomnolence may be assessed by the Epworth Sleepiness Scale, the Stanford Sleepiness Scale, or the Idiopathic Hypersomnia Severity scale. The Epworth Sleepiness Scale (ESS) evaluates overall daytime sleepiness. The questionnaire asks respondents to rate how likely they are to fall asleep in eight different situations representing a moment of relative inactivity, such as a nap in the afternoon or sitting in a car stopped in traffic. Using a scale of 0-3 (with 0 meaning “would never doze” and 3 meaning “high chance of dozing”), respondents rate their likelihood of falling asleep. Scoring ranges from 0-24; the higher the score, the higher the severity of daytime sleepiness. A cut-off score of 10 identifies daytime sleepiness at a potentially clinical level. Treatment success may be indicated (i) by a decrease of the score, preferably (ii) by a decrease to 10 or below.

The Stanford Sleepiness Scale is a patient/subjective measure of sleepiness, evaluating sleepiness at specific moments in time. Consisting of only one item, the scale requires respondents to select one of seven statements best representing their current level of perceived sleepiness. A scale from 1 (=Feeling active and vital; alert; wide awake) to 7 (=Almost in reverie; sleep onset soon; lost struggle to remain awake) is used to assess the level of sleepiness. Treatment success may be indicated by a decrease of the score. Parasomnias may be evaluated by the Paris Arousal Disorders Severity Scale (PADSS). The Paris Arousal Disorders Severity Scale (PADSS) is a self-rating scale listing parasomniac behaviours, assessing their frequency and includes an evaluation of consequences. Treatment success may be indicated (i) by a decrease of the score, preferably (ii) by a decrease to below the cut-off value.

A common questionnaire that assesses sleep-related breathing disorders is the Berlin Questionnaire. An appropriate recall period may also be chosen. Treatment success may be indicated by a decrease of the score. A common questionnaire that assesses sleep-related movement disorders is the International Restless Legs Syndrome Study Group Rating Scale. The 10-item questionnaire asks respondents to use Likert-type ratings to indicate how acutely the disorder has affected them over the course of the past week. Questions may be divided into one of two categories: disorder symptoms (nature, intensity, and frequency) and their impact (sleep issues, disturbances in daily functioning, and resultant changes in mood. Each of the ten questions requires respondents to rate their experiences with RLS on a scale from 0 to 4, with 4 representing the most severe and frequent symptoms and 0 representing the least. Total scores may range from 0 to 40. As a brief scale with excellent psychometric qualities, the instrument may be suitable for a variety of research and clinical purposes, including screening and assessment of treatment outcomes. Treatment response may be assessed by a decrease of the score.

Bipolar disorder (BD) has various aspects and is characterised by various symptoms. The predominant psychopathology is depression, and the presentation of a patient/subject experiencing a depressive phase may initially result in the diagnosis of that patient/subject as having major depressive disorder (MDD). However, BD possesses multiple characteristics that define it as distinct from the latter even during the depressive phase.

Of particular interest here are the symptoms that are more strongly associated with BD compared to other psychiatric disorders, as these are the metrics against which patient/subject treatment is assessed. Notwithstanding that many symptoms may be said to straddle multiple disorders, much work has been done to identify several symptoms that present strongly in BD patients/subjects: sleep disturbance, psychomotor retardation (reduced energy and activity and reduced motivation), negative thinking (feelings of worthlessness; helplessness and hopelessness; guilt), anxiety, cognitive dysfunction (impaired concentration and memory) and social/emotional withdrawal or detachment (anhedonia, emotional withdrawal and affective flattening). Characteristic symptoms further include suicidal ideation. Still further, characteristic symptoms include mixed symptoms (psychotic symptoms; irritability; lability; increased motor drive; increased speech; agitation).

Clinical assessment tools such as the Bipolar Depression Rating Scale (BDRS) have been developed and validated for use in BD, which take into account these symptoms. The Bipolar Depression Rating Scale (BDRS) is designed to measure the severity of depressive symptoms in bipolar depression. The BDRS is validated for clinical use by trained raters. Based on a clinical interview, the BDRS items rate the severity of depressive and/or mixed symptoms expressed by patients/subjects currently and during the past few days. If there is a discordance between symptoms currently and the last few days, the rating should reflect current symptoms. The scale contains 20 questions and the maximum score possible is 60. Higher scores indicate greater severity.

The questions address depressed mood; sleep disturbance; appetite disturbance; reduced social engagement; reduced energy and activity; reduced motivation; impaired concentration and memory; anxiety; anhedonia; affective flattening; feelings of worthlessness; feelings of helplessness and hopelessness; suicidal ideation; feelings of guilt; psychotic symptoms; irritability; lability; increased motor drive; increased speech; agitation. Each of these aspects is assessed and assigned a score of 0, 1, 2 or 3.

Depressed mood is scored as 0 if there is no self-reported and/or observed depression as evidenced by gloom, sadness, pessimism, hopelessness, and helplessness; 1 (mild) in case of brief or transient periods of depression, or mildly depressed mood; 2 (moderate) in case a depressed mood is clearly but not consistently present and other emotions are expressed, or depression is of moderate intensity; 3 (severe) in case of pervasive or continuous depressed mood of marked intensity.

Sleep disturbance (sleep dysregulation) is assessed based on the change in total amount of sleep over a 24-hour cycle, rated independent of the effect of external factors. It may either take the form of insomnia (reduction in total sleep time) or the form of hypersomnia (increase in total sleep time, inclusive of daytime sleep). The rating for insomnia involves scores of 0 (no reduction in total sleep time); 1 (mild; reduction up to 2 hours); 2 (moderate; 2-4 hours); 3 (severe; more than 4 hours). The alternative rating for hypersomnia involves scores of 0 (no increase in total sleep time, inclusive of daytime sleep); 1 (mild; less than 2 hours, or normal amount but nonrestorative); 2 (moderate; 2-4 hours); 3 (severe; greater than 4 hours).

Appetite disturbance is assessed based on the change in appetite and food consumption, rated independent of the effect of external factors. It may either take the form of loss of appetite or the form of increase in appetite. The rating for loss of appetite involves scores of 0 (no change in appetite and food consumption); 1 (mild; no change in food intake, but has to push self to eat or reports that food has lost taste); 2 (moderate; some decrease in food intake); 3 (marked decrease in food intake, hardly eating). The alternative rating for increase in appetite involves scores of 0 (no change in appetite and food consumption); 1 (mild; no change in food intake, but increased hunger); 2 (moderate; some increase in food intake, e.g., comfort eating); 3 (marked increase in food intake or cravings).

Reduced social engagement is scored as 0 if there are no patient/subjective reports of reduced social and interpersonal engagement or interactions; 1 (mild) in case of slight reduction in social engagement with no impairment in social or interpersonal function; 2 (moderate) in case of clear reduction in social engagement with some functional sequelae, e.g., avoiding some social engagements or conversations; and 3 (severe) in case of marked reduction in social interaction or avoidance of almost all forms of social contact, e.g., refusing to answer the phone or see friends or family.

Reduced energy and activity is scored as 0 if there is no reduced energy, drive or goal directed behaviour; 1 (mild) in case of ability to engage in usual activities but with increased effort; 2 (moderate) in case of significant reduction in energy leading to reduction of some role-specific activities; and 3 (severe) in case of leaden paralysis or cessation of almost all role specific activities, (e.g., spending excessive time in bed, avoiding answering the phone, poor personal hygiene).

Reduced motivation is scored as 0 if there are no reports of patient/subjective reduction in drive, motivation, and consequent goal directed activity; 1 (mild) in case of a slight reduction in motivation with no reduction in function; 2 (moderate) in case of a reduced motivation or drive with significantly reduced volitional activity or requiring substantial effort to maintain usual level of function; and 3 (severe) in case of reduced motivation or drive such that goal directed behaviour or function is markedly reduced.

Impaired concentration and memory are scored as 0 if there are no patient/subjective reports of reduced attention, concentration, or memory, and consequent functional impairment; 1 (mild) in case of slight impairment of attention, concentration, or memory with no functional impairment; 2 (moderate) in case of significant impairment of attention, concentration, or forgetfulness with some functional impairment; 3 (severe) in case of marked impairment of concentration or memory with substantial functional impairment, e.g., unable to read or watch TV).

Anxiety is scored as 0 if there are no patient/subjective reports of worry, tension, and/or somatic anxiety symptoms e.g., tremor, palpitations, dizziness, light-headedness, pins and needles, sweating, dyspnoea, butterflies in the stomach, or diarrhoea; 1 (mild; transient worry or tension about minor matters); 2 (moderate; significant anxiety, tension, or worry, or some accompanying somatic features); 3 (severe; marked continuous anxiety, tension, or worry that interferes with normal activity; or panic attacks).

Anhedonia is scored as 0 (no patient/subjectively reduced ability to experience pleasure in usual activities); 1 (mild; slight reduction in pleasure from usually pleasurable activities); 2 (moderate; significant reduction in pleasure from usually pleasurable activities; some pleasure from isolated activities retained); or 3 (severe; complete inability to experience pleasure). Affective flattening is scored as 0 if there is no patient/subjective sense of reduced intensity or range of feelings or emotions; 1 (mild) in case of slight constriction of range of affect, or transient reduction in range or intensity of feelings; 2 (moderate) in case of significant constriction of range or intensity of feelings with preservation of some emotions, e.g., inability to cry; and 3 (severe) in case of marked and pervasive constriction of range of affect or inability to experience usual emotions.

Feelings of worthlessness (also simply referred to as worthlessness) are scored as 0 (no patient/subjective sense, or thoughts, of decreased self-value or self-worth); 1 (mild; slight decrease in sense of self-worth); 2 (moderate; some thoughts of worthlessness and decreased self-worth) 3 (severe; marked, pervasive, or persistent feelings of worthlessness, e.g., feels others better off without them, unable to appreciate positive attributes).

Feelings of helplessness and hopelessness (also simply referred to as helplessness and hopelessness) characterise the patient/subjective sense of pessimism or gloom regarding the future, inability to cope, or sense of loss of control. If this is absent, the score is 0. The score is 1 (mild) in case of occasional and mild feelings of not being able to cope as usual, or pessimism; it is 2 (moderate) in case the patient/subject often feels unable to cope, or has significant feelings of helplessness or hopelessness which lift at times; it is 3 (severe) if there are marked and persistent feelings of pessimism, helplessness, or hopelessness.

Suicidal ideation relates to thoughts or feelings that life is not worthwhile; thoughts of death or suicide and is scored 0 if such thoughts are absent; 1 (mild) in case of thoughts that life is not worthwhile or is meaningless; 2 (moderate) in case of thoughts of dying or death, but with no active suicide thoughts or plans; 3 (severe) in case of thoughts or plans of suicide. Feelings of guilt (also simply referred to as guilt) are scored as 0 if there is no patient/subjective sense of self blame, failure, or remorse for real or imagined past errors; 1 (mild) in case of slight decrease in self-esteem or increased self-criticism; 2 (moderate) in case of significant thoughts of failure, self-criticism, inability to cope, or ruminations regarding past failures and the effect on others; able to recognise as excessive; 3 (severe) in case of marked, pervasive, or persistent guilt, e.g., feelings of deserving punishment; or does not clearly recognise as excessive.

Psychotic symptoms are scored as 0 if overvalued ideas, delusions, or hallucinations are absent; 1 (mild) in case of mild overvalued ideas, e.g., self-criticism or pessimism without clear effect on behaviour; 2 (moderate) in case of significant overvalued ideas with clear effect on behaviour, e.g., strong guilt feelings, clear thoughts that others would be better off without them; 3 (severe) in case of clear psychotic symptoms, e.g., delusions or hallucinations.

Irritability reports uncharacteristic patient/subjective irritability, short fuse, easily angered, manifested by verbal or physical outbursts and is scored 0 if absent; 1 (mild) in case of slight patient/subjective irritability which may not be overtly present; 2 (moderate) in case of verbal snappiness and irritability that is clearly observable in the interview; 3 (severe) in case of reports of physical outbursts, e.g., throwing/breaking objects, or markedly abusive verbal outbursts. Lability is scored 0 if there are no observed mood lability or reported mood swings. It is scored 1 (mild) in case of patient/subjective reports of mild increase in mood lability; 2 (moderate) if mood lability is clearly observable, moderate in intensity; 3 (severe) in case of marked and dominant mood lability, frequent or dramatic swings in mood.

Increased motor drive relates to patient/subjective reports and objective evidence of increased motor drive and motor activity. It is scored 0 in case of normal motor drive: 1 (mild) in case of a slight increase in drive, not observable in the interview; 2 (moderate) in case of clear and observable increase in energy and drive; 3 (severe) if there is a marked or continuous increase in drive. Increased speech relates to an observed increase in either the rate or quantity of speech, or observed flight of ideas. This item is scored 0 if such observations are absent; 1 (mild) if there is a slight increase in the rate or quantity of speech; 2 (moderate) in case of racing thoughts, or if the patient/subject is significantly more talkative, clearly distractible, or in case of some circumstantiality; wherein this does not impede the interview; 3 (severe) in case of flight of ideas; which interferes with the interview.

Agitation is scored 0 if there is no observed restlessness or agitation; 1 (mild) in case of slight restlessness; 2 (moderate) in case of clear increase in level of agitation; 3 (severe) in case of marked agitation, e.g., near continuous pacing or wringing hands. While a higher score on the BDRS scale indicates more severe disease, there are no generally accepted limits for when a patient/subject is to be considered moderately or severely ill. BDRS score ranges used herein for indicating the severity of depressive episodes in patients/subjects with bipolar disorder are 13-18 for “mildly ill”, 19-23 for “moderately ill”, 24-36 for “markedly ill”, 37-39 for “severely ill”, and * 40 for “extremely ill”. Various other scales are also useful to assess the severity of disease as well as the clinical outcome of treatments.

Anxiety is sometimes defined as an “apprehensive anticipation of future danger or misfortune accompanied by a feeling of dysphoria or somatic symptoms of tension”. Anxiety is characterised by an intense, excessive, and persistent worry and fear about a situation that is only patient/subjectively seen as menacing and is often accompanied by muscular tension, restlessness, fatigue, inability to catch one's breath, tightness in the abdominal region, nausea, and problems in concentration.

In anxiety disorders or other mental or nervous system disorders associated with anxiety, the feelings of anxiety are difficult to control and interfere with daily activities. Anxiety is a core feature of anxiety disorders, including separation anxiety disorder, specific phobia, social anxiety disorder (social phobia), panic disorder, generalised anxiety disorder (GAD), agoraphobia, and substance/medication-induced anxiety disorder. Anxiety is moreover associated with several other mental and nervous system disorders. Anxiety is also associated with sleep disturbance.

Several rating scales to assess anxiety are known on the art, and anxiety symptoms are furthermore assessed as part of various rating scales used to assess mental and nervous system disorders. The Hamilton Anxiety Rating Scale (HAM-A) is designed to assess anxiety symptoms. The scale is clinician/physician-administered. It has 14 items which may be divided into a group of psychic items (1-6 and 14) measuring in particular mental agitation and psychological distress and into a group of somatic items (items 7-13) measuring in particular physical complaints related to anxiety. Each item is rated by the interviewer on a scale from 0 to 4: 0=Not present, 1=Mild, 2=Moderate, 3=Severe, 4=Very severe. A total score is obtained by summing the 14 items. The total score range is 0-56. Higher scores indicate more anxiety. A score ≤7 is considered to represent no or minimal anxiety; a score of 8-14 mild anxiety; a score of 15-23 moderate anxiety; a score ≥24 severe anxiety.

The Beck Anxiety Inventory (BAI) is a 21-item self-report questionnaire developed to assess anxiety, with a focus on somatic symptoms. The items are rated on a four-point Likert scale ranging from zero (not at all) to three (severely: I could barely stand it). The total score ranges from 0 to 63. Subthreshold anxiety as the term is used herein in particular means that the patient/subject has a Hamilton Rating Scale for Anxiety (HAM-A) score of at least 9 but of less than 18 and/or a Beck Anxiety Inventory (BAI) score of at least 1 1 but of less than 16.

Negative thinking or individual aspects thereof, such as worthlessness, helplessness and hopelessness, and guilt, may be evaluated by different instruments, such as questionnaires or scales. Questionnaires assess the mental status of a patient/subject based on observations made by the patient/subject himself/herself, caregivers or the clinician/physician administering the questionnaire. Questionnaires used to assess whether a patient/subject suffers from a particular mental or nervous system disorder may comprise items related to negative thinking. Instruments evaluating relevant aspects of negative thinking include, for example, the State Shame and Guilt Scale (SSGS), the Positive and Negative Affect Schedule-Expanded Form (PANAS-X) or the State Hope Scale (SHS).

The State Shame and Guilt Scale (SSGS) is a self-rating scale of in-the-moment (state) feelings of shame, and guilt experiences. It comprises two subscales, a shame and a guilt subscale. The shame subscale comprises items 1, 3, 5, 7, 9. The guilt subscale comprises items 2, 4, 6, 8, 10. All items are scored in a positive direction and are rated on a 5-point Likert scale. It contains some statements which may or may not describe how the patient/subject is feeling right now. A higher score indicates a more intense feeling of shame or guilt. The Positive and Negative Affect Schedule-Expanded Form (PANAS-X) is a 60-item, expanded version of the PANAS. The PANAS-X measures 11 specific affects: Fear, Sadness, Guilt, Hostility, Shyness, Fatigue, Surprise, Joviality, Self-Assurance, Attentiveness, and Serenity. The PANAS-X thus provides for mood measurement at two different levels. The basic negative emotion scales are fear, hostility, guilt and sadness, while the scale of guilt encompasses six items: guilty, ashamed, blameworthy, angry at self, disgusted with self, dissatisfied with self. Each answer should be scored as 1=very slightly or not at all; 2=a little; 3=moderately; 4=quite a bit; or 5=extremely. However, investigators facing more severe time constraints may select and assess only those scales that are most relevant to their research.

More intense feelings of guilt are reflected by a higher score on the guilt scale. The PANAS-X is simple and easy to administer. Most patients/subjects complete the entire 60-item schedule in 10 minutes or less. This scale consists of a number of words and phrases that describe different feelings and emotions. While it should be indicated to what extent the patient/subject has felt this way during the past few weeks, it has been found that the trait scores on the PANAS-X scales are stable over time, including “at the present moment”, “today” and during “the past few days”, indicating that an appropriate shorter recall period may be applied.

The State Hope Scale (SHS) has three agency and three pathways items to which respondents describe themselves in terms of how they are “right now.” The agency subscale score is derived by summing items 2, 4 and 6, relating to the perceived capacity to use one's pathways to reach desired goals; the pathways subscale score is derived by adding the items 1, 3 and 5, relating to thinking that is used to identify possible ways to achieve a goal. The total State Hope Scale score is derived by summing the three agency and the three pathways items. Scores may range from a low of 6 to a high of 48, wherein higher hope is reflected by a higher score on this scale. Negative thinking or aspects thereof are also reflected in other scales such as HAM-D, the MADRS, the BPRS or the BDRS, wherein relevant items thereof may be commonly applicable for assessing negative thinking or aspects thereof.

Cognition includes the skills needed for thinking, remembering, paying attention, and solving problems. Loss or decline of these skills leads to cognitive dysfunction, a term used herein to refer to a deficit in, or an impairment of, any domain of cognition. Cognitive dysfunction may be one of the manifestations of a patient's underlying condition.

The DSM-5 defines six key domains of cognitive function, namely complex attention, executive function, learning and memory, language, perceptual-motor function, and social cognition. Cognitive dysfunction may impact one or more of those domains. In fact, cognitive abilities are highly interrelated, and it is not unusual that more than one domain is affected. For instance, the domain complex attention has the subdomains sustained attention (commonly referred to as ‘concentration’ or ‘focus’), divided attention, selective attention, and processing speed.

Thus, complex attention evidently encompasses aspects which are critical for a variety of cognitive tasks, such as executive function and learning and memory. Cognitive control or executive function is intrinsically attentional. Also, perception, and decision-making are profoundly influenced by attention abilities. As a consequence, attention is not only tested for in isolation, but for example, also tested by cognitive control tasks/executive function. If attention is impaired, other types of cognitive abilities will likely also be impaired. Before language may be comprehended, visual-spatial relationships perceived, information remembered or problems solved, the stimuli must be attended to.

Cognitive dysfunction, which term herein means an acquired condition and thus represents a decline from a previously attained level of functioning, may be associated with various processes. In a healthy individual, certain cognitive abilities, such as accumulated knowledge and vocabulary, are maintained upon ageing and may even improve over time. However, even in the absence of any pathological condition, ageing leads to declines in abilities like thinking abstractly, reasoning, and decision-making. These deteriorations are linked to underlying age-related deficits in processing speed, attention, memory, and executive function, which are indicative of cognitive ageing.

Independent of normal ageing, cognitive dysfunction may be associated with one or more conditions or disorders, such as a mental disorder or a nervous system disorder or some other medical conditions. Mental or nervous system disorders which lead to, or are associated with, cognitive dysfunction include disorders characterised by depressive episodes, for example, Major Depressive Disorder (MDD), Postpartum Depression (PPD), Persistent Depressive Disorder, Seasonal Affective Disorder and Bipolar Disorder (BD), such as Bipolar I Disorder and Bipolar II Disorder; Anxiety Disorder, for example Separation Anxiety Disorder, Agoraphobia, Generalised Anxiety Disorder (GAD), Social Anxiety Disorder (SAD), Panic Disorder, Phobia, and Substance/Medication Induced Anxiety Disorder; Somatic Symptom Disorder; Obsessive Compulsive and Related Disorders, for example, Obsessive Compulsive Disorder (OCD) and Body Dysmorphic Disorder (BDD); PostTraumatic Stress Disorder (PTSD); Pain Disorders, for example Chronic Pain, Fibromyalgia and Migraine; Mental and Behavioural Disorders due to Psychoactive Substance Use, for example, Substance Use Disorder (SUD); Psychotic Disorders, for example Schizophrenia; Huntington's Disease; Parkinson's Disease; Dementia, for example Alzheimer's Dementia (AD), Parkinson's Disease Dementia (PDD), Dementia with Lewy Bodies, Vascular Dementia, FrontoTemporal Dementia; Eating Disorders; Attention Deficit Hyperactivity Disorder (ADHD); Personality Disorders, for example Schizotypal Personality Disorder and Borderline Personality Disorder; Chronic Fatigue Syndrome; one or more conditions or disorders, such as a mental disorder or a nervous system disorder associated with HIV, Traumatic Brain Injury or Post COVID Condition. The cognitive dysfunction may also occur in a patient/subject suffering from sleep disturbance, for instance, insomnia.

Cognitive dysfunction furthermore occurs in disorders showing symptoms characteristic of a neurocognitive disorder that cause clinically significant distress or impairment in social, occupational, or other important areas of functioning but do not meet the full criteria for any aetiology-related disorder. Cognitive dysfunction may take the form of a neurocognitive disorder. Mild neurocognitive disorder, also referred to as mild cognitive impairment, is characterised by a modest cognitive decline from a previous level of performance in one or more of the cognitive domains. Affected patients/subjects are still able to stay independent and do daily tasks. However, the patient/subject usually functions at a suboptimal level. Everyday tasks become more effortful owing to the engagement of compensatory strategies to maintain independence. In major neurocognitive disorder, a significant cognitive decline from a previous level of performance in one or more of the cognitive domains is observed. The cognitive deficits interfere with independence in everyday activities.

Cognitive dysfunction may be evaluated by questionnaires or by neuropsychological assessments. Questionnaires assess the mental status of a patient/subject based on observations made by the patient/subject himself, caregivers or the clinician/physician administering the questionnaire. Questionnaires used to assess whether a patient/subject suffers from a particular mental or nervous system disorder may comprise items related to cognitive function. A neuropsychological assessment is a process by which a person's cognitive, psychological/emotional and behavioural functioning is comprehensively evaluated. A core part of neuropsychological assessment is the administration of neuropsychological tests for the formal assessment of cognitive function.

Performance in these tests is compared with norms appropriate to the patient's age, educational attainment, and cultural background. Testing often uses a set of performance-based questions, also known as a neuropsychological test battery. The abilities tested include language processing, visuospatial processing, attention/concentration, verbal learning and memory, visual learning and memory, executive functions, speed of processing, and sensory-perceptual functions.

Common tests that assess cognitive dysfunction are the Montreal Cognitive Assessment (MoCA), the Mini-Mental State Examination (MMSE), the Mini-Cog™, the Screen for Cognitive Impairment in Psychiatry (SCIP), and the MATRICS Consensus Cognitive Battery (MCCB). The Montreal Cognitive Assessment (MoCA) is a widely used screening assessment for detecting cognitive impairment. It assesses different cognitive domains: short-term memory; visuospatial abilities; executive functions; attention, concentration and working memory; language; orientation to time and space. The total possible score is 30 points; a score of 26 or above is considered normal; a score of 18-25 is considered mild cognitive impairment, a score of 10-17 is considered moderate cognitive impairment and a score less than 10 is considered severe cognitive impairment.

The Mini-Mental State Examination (MMSE) is an 11-question measure that tests five areas of cognitive function: orientation, registration, attention and calculation, recall, and language. The maximum score is 30. The raw score may also need to be corrected for educational attainment and age. Four cut-off levels are employed herein to classify the severity of cognitive impairment: 24-30 means no cognitive impairment; 19-23 means mild cognitive impairment; 10-18 means moderate cognitive impairment; and * 9 means severe cognitive impairment. Used repeatedly, the MMSE is suitable to measure changes in cognitive status. The Mini-Cog™ is a short cognitive impairment screening questionnaire. It combines a 3-word recall with a clock drawing test. The clock drawing test assesses many cognitive areas that may be affected, such as executive function, visuospatial abilities, motor programming, and attention. One point is given for each of the three words correctly recalled after performing the clock drawing test; a correctly drawn clock is worth two points. A score of <4 indicates cognitive impairment.

The Screen for Cognitive Impairment in Psychiatry (SCIP) is a well-evaluated screening instrument for the examination of cognitive performance in psychiatric patients/subjects. The SCIP consists of five subscales: verbal learning test-immediate (VLT-I), working memory test (WMT), verbal fluency test (VFT), verbal learning test-delayed (VLT-D) and processing speed test (PST). There are three different test forms to facilitate test repetition and therefore reducing learning effect. Subscale scores are calculated for each of the five tests, and a total score is calculated from the sum of the subscale scores. A total score of less than 70 indicates cognitive dysfunction.

Cognitive dysfunction may also be assessed by the MCCB (MATRICS Consensus Cognitive Battery) or by one or more of the various subtests. The subtests are: Trail Making Test, Part A (testing speed of processing); Brief Assessment of Cognition in Schizophrenia, symbol coding subtest (speed of processing); Hopkins Verbal Learning Test-Revised, immediate recall, three learning trials only (verbal learning); Wechsler Memory Scale, 3rd ed., spatial span subtest (working memory (nonverbal)); Letter-Number Span test (working memory (verbal)); Neuropsychological Assessment Battery, mazes subtest (reasoning and problem solving); Brief Visuospatial Memory Test-Revised (visual learning); Category fluency test, animal naming (speed of processing); Mayer-Salovey-Ca-ruso Emotional Intelligence Test, managing emotions branch (social cognition); and Continuous Performance Test, Identical Pairs version (attention/vigilance). The test battery is appropriate to measure cognitive change. Further tests are the Verbal Recognition Memory (VRM) test, the Rapid Visual information Processing (RVP) test, the Spatial Working Memory (SWM) test and the Digit Symbol Substitution Test (DSST).

Postpartum depression (PPD) is a complex mix of physical, emotional, and behavioural changes that happen in some women after giving birth. PPD is also known as major depressive disorder with peripartum onset. According to DSM-5 (Diagnostic and Statistical Manual of Mental Disorders, 5th Edition) criteria, PPD is diagnosed when major depressive disorder (MDD) symptoms begin during pregnancy or within four weeks of delivery. A patient/subject treated according to the present invention is preferably a woman diagnosed with PPD and >4 weeks postpartum. Further, the patient/subject will preferably be * 9 months postpartum. Depressive aspects of PPD may be assessed by the HAM-D or the MADRS score. The Edinburgh Postnatal Depression Scale (EPDS) may also be used.

The Montgomery-Asberg Depression Rating Scale (MADRS) is a ten-item diagnostic questionnaire used to measure the severity of depressive episodes in patients/subjects with mood disorders (Montgomery, S. A., & Asberg, M. (1979). A new depression scale designed to be sensitive to change. The British Journal of Psychiatry 134, p. 382). It was designed as an adjunct to the Hamilton Rating Scale for Depression (HAM-D), which would be more sensitive to the changes brought on by antidepressants and other forms of treatment. Higher MADRS score indicates more severe depression. The items considered are apparent sadness; reported sadness; inner tension; reduced sleep; reduced appetite; concentration difficulties; lassitude; inability to feel; pessimistic thoughts; and suicidal thoughts, and each item yields a score of 0 to 6. The overall score ranges from 0 to 60.

A patient/subject may suffer from moderate or severe PPD as indicated by a Montgomery-Asberg Depression Rating Scale (MADRS) score of 20 or more or by a Hamilton Depression Rating Scale (HAM-D) score of 16 or more. It is further considered that the patient/subject may suffer from severe PPD as indicated by a Montgomery-Asberg Depression Rating Scale (MADRS) score of 35 or more or by a Hamilton Depression Rating Scale (HAM-D) score of 27 or more. The patient/subject may be diagnosed with a treatment-resistant form of PPD. A patient/subject treated according to the invention may have a Montgomery-Asberg Depression Rating Scale (MADRS) score of 20 or more or a 17-item Hamilton Depression Rating Scale (HAM-D) score of 16 or more.

Further, a patient/subject treated according to the invention may have a MADRS score of 28 or more or a HAM-D score of 22 or more. Still further, a patient/subject treated according to the invention may have a MADRS score of 35 or more or a HAM-D score of 25 or more. In addition to the above, PPD compromises maternal functioning. In particular the first year after childbirth marks a critical window for both mother and child. In most cases, mothers are the primary caregivers and are, therefore, responsible for the majority of the work related to infant care tasks.

Maternal functioning includes aspects of maternal competence relating to interactions with the infant(s) as well as maternal self-care. Maternal functioning, including the emotional aspect of mothering, is also important for the child's development. In fact, the quality of mother-child interaction in the year after birth affects infant development. High levels of maternal functioning are likely to correlate with positive infant development outcomes. Likewise, impaired functioning in the postpartum period might impede optimal infant development.

The Barkin Index of Maternal Functioning (BIMF) was designed to measure functioning in the year after childbirth. The BIMF is a 20-item self-report measure of functioning. Each item is assigned a score between 0 and 6 so that the maximum total score is 120. The higher the score, the better maternal functioning is rated. The BIMF identifies the key functional domains of a mother during the postnatal period as: self-care, infant care, mother-child interaction, psychological wellbeing of the mother, social support, management, and adjustment.

A BIMF score of 95 or below is considered herein as representing slightly compromised maternal functioning, score of 80 or below is considered herein as representing compromised maternal functioning, a score of 65 or below is considered herein as representing severely compromised maternal functioning. The invention in particular allows improving maternal functioning in patients/subjects having a score of 80 or below before treatment and in patients/subjects having a score of even 65 or below.

Symptoms such as anhedonia; emotional withdrawal and affective flattening are clustered together here as social/emotional withdrawal or detachment. Reduced social engagement is a further aspect associated with social/emotional withdrawal or detachment.

Anhedonia is the inability to experience pleasure. The patient/subject does not suffer from anhedonia if there is patient/subjectively no reduced ability to experience pleasure in usual activities. Anhedonia is mild in the case of slight reduction in pleasure from usually pleasurable activities; moderate in the case of significant reduction in pleasure from usually pleasurable activities or some pleasure from isolated activities retained; or severe in the case of complete inability to experience pleasure.

Anhedonia comprises consummatory (or liking) and anticipatory (or wanting) components. Consummatory pleasure refers to the “in the moment” pleasure experienced by the patient/subject directly engaged in an enjoyable activity, whereas anticipatory pleasure refers to the experience of pleasure related to future activities. Affective flattening characterises the patient/subjective sense of reduced intensity or range of feelings or emotions. The patient/subject does not show affective flattening if there is no sense of reduced intensity or range of feeling or emotions. It is mild in the case of slight constriction of range of affect, or transient reduction in range or intensity of feelings; moderate in the case of significant constriction of range or intensity of feelings with preservation of some emotions, e.g., inability to cry; and severe in the case of marked and pervasive constriction of range of affect or inability to experience usual emotions.

Emotional withdrawal or detachment is an inability or unwillingness to connect with other people on an emotional level. For example, the BPRS contains an item relating to emotional withdrawal, which is characterised as the deficiency in the patient/subject's ability to relate emotionally during the interview situation. According to the description of this BPRS item, there is no emotional withdrawal if there is no lack of emotional involvement shown by occasional failure to make reciprocal comments, occasionally appearing preoccupied, or smiling in a stilted manner, but spontaneously engages the interviewer most of the time.

There is a mild form of emotional withdrawal if there is a lack of emotional involvement shown by noticeable failure to make reciprocal comments, appearing preoccupied, or lacking in warmth, but responds to the interviewer when approached. It is moderate if the emotional contact is not present much of the interview because the patient/subject does not elaborate responses, fails to make eye contact, does not seem to care if the interviewer is listening, or may be preoccupied with psychotic material. It is moderately severe if, in addition, emotional contact is not present most of the interview. Severe forms are present if emotional participation is actively avoided by the patient/subject or if the patient/subject is frequently unresponsive or responds with yes/no answers or responds with only minimal affect. It is extremely severe if the patient/subject consistently avoids emotional participation, is unresponsive or responds with yes/no answers or may leave during the interview or just not respond at all.

Reduced social engagement characterises patient/subjective reports of reduced social and interpersonal engagement or interactions. There is no reduced social engagement if there are no reports of reduced social and interpersonal engagement or interactions. It is mild in the case of slight reduction in social engagement with no impairment in social or interpersonal function; moderate in the case of clear reduction in social engagement with some functional sequelae, e.g., avoiding some social engagements or conversations; and severe in the case of marked reduction in social interaction or avoidance of almost all forms of social contact, e.g., refusing to answer the phone or see friends or family.

Social/emotional withdrawal or detachment may be associated with one or more conditions or disorders, such as a mental disorder or a nervous system disorder or some other medical conditions. Mental or nervous system disorders which lead to, or are associated with, social/emotional withdrawal or detachment include disorders characterised by depressive episodes, for example Major Depressive Disorder (MDD), Bipolar Disorder (BD), such as Bipolar I Disorder and Bipolar II Disorder, Postpartum Depression (PPD), Seasonal Affective Disorder and Persistent Depressive Disorder; Anxiety Disorders, for example Generalised Anxiety Disorder (GAD) and Social Anxiety Disorder (SAD); Obsessive Compulsive and Related Disorders, for example, Obsessive Compulsive Disorder (OCD) and Body Dysmorphic Disorder (BDD); PostTraumatic Stress Disorder (PTSD); Pain Disorders, for example Chronic Pain and Fibromyalgia; Mental and Behavioural Disorders due to Psychoactive Substance Use, for example Substance Use Disorder (SUD); Psychotic Disorders, for example Schizophrenia; Dementia, for example Alzheimer's Dementia (AD); Dementia with Lewy Bodies (DLB); Vascular Dementia and Fronto-Temporal Dementia (FTD); Parkinson's Disease (PD); Eating Disorders; Autism Spectrum Disorder (ASD); Attention Deficit Hyperactivity Disorder (ADHD); and Personality Disorders, for example Schizotypal Personality Disorder and Borderline Personality Disorder (BPD).

The social/emotional withdrawal or detachment may also occur in a patient/subject suffering from sleep disturbance, for instance, insomnia. The social/emotional withdrawal or detachment may also occur in a patient/subject suffering from medical health conditions leading to an associated mental or nervous system condition including Traumatic Brain Injury (TBI).

Social/emotional withdrawal or detachment or individual aspects thereof, such as anhedonia, emotional withdrawal and affective flattening, may be evaluated by different instruments, such as questionnaires or scales. Questionnaires assess the mental status of a patient/subject based on observations made by the patient/subject himself/herself, caregivers or the clinician/physician administering the questionnaire. Questionnaires used to assess whether a patient/subject suffers from a particular mental or nervous system disorder may comprise items related to social/emotional withdrawal or detachment.

The Snaith-Hamilton Pleasure Scale (SHAPS) is a 14-item scale that measures anhedonia, i.e., the inability to experience pleasure. The items cover the domains of: social interaction, food and drink, sensory experience, and interest/pastimes. A score of 2 or less constitutes a “normal” score, while an “abnormal” score is defined as 3 or more. Each item has four possible responses: strongly disagree, disagree, agree, or strongly agree. Either of the “disagree” responses score one point, and either of the “agree” responses score 0 points. Thus, the final score ranges from 0 to 14. The SHAPS has adequate construct validity and satisfactory test-retest reliability. High internal consistency has also been reported. The SHAPS has been used for measuring anhedonia in depression, but it is also frequently used to assess anhedonia in other patient/subject groups.

In principle, the SHAPS measures hedonic tone during the last few days with 14 hypothetically formulated items. However, due to the hypothetical nature of the items an appropriate shorter recall period may also be applied for an earlier assessment time point. Alternatively or additionally, the Dimensional Anhedonia Rating Scale (DARS) measuring interest, motivation, effort and consummatory pleasure across four domains: hobbies, food/drink, social activities and sensory experience may be used for the assessment of anhedonia. It comprises 17 items assessing state anhedonia right now. The DARS is rated on a five-point Likert scale from 0 (not at all) to 4 (very much), higher values indicating less anhedonia. All items are summed up to a total score in the range of 0 to 68. For each of the four hedonic domains, hobbies (four items, sum score 0-16), food/drink (four items, sum score 0-16), social activities (four items, sum score 0-16) and sensory experiences (5 items, sum score 0-20), patients/subjects/patient/subjects are asked to provide two or three of their own favourite examples.

The Personality Inventory for DSM-5 (PID-5)—Adult is a 220 item self-rated personality trait assessment scale for adults age 18 and older. It assesses 25 personality trait facets including Anhedonia, Anxiousness, Attention Seeking, Callousness, Deceitfulness, Depressivity, Distractibility, Eccentricity, Emotional Lability, Grandiosity, Hostility, Impulsivity, Intimacy Avoidance, Irresponsibility, Manipulativeness, Perceptual Dysregulation, Perseveration, Restricted Affectivity, Rigid Perfectionism, Risk Taking, Separation Insecurity, Submissiveness, Suspiciousness, Unusual Beliefs and Experiences, and Withdrawal, with each trait facet consisting of 4 to 14 items.

The trait facet Anhedonia contains the items 1, 23, 26, 30R, 124, 155R, 157, 189 (reverse scored items are marked with the letter “R”), the trait facet Withdrawal contains the items 10, 20, 75, 82, 136, 146, 147, 161, 182, 186 and the trait facet Intimacy Avoidance contains the items 89, 97R, 108, 120, 145, 203. These three trait facets may be combined to yield the broader trait domain designated Detachment. The measure is completed by the individual prior to a visit with the clinician/physician. Each item asks the individual to rate how well the item describes him or her generally. Each item on the measure is rated on a 4-point scale. The response categories for the items are 0=very false or often false; 1=sometimes or somewhat false; 2=sometimes or somewhat true; 3=very true or often true. For items 7, 30, 35, 58, 87, 90, 96, 97, 98, 131, 142, 155, 164, 177, 210, and 215, the items are reverse-coded prior to entering into scale score computations.

The scores on the items within each trait facet should be summed and entered in the appropriate raw facet score box. In addition, the clinician/physician is asked to calculate and use average scores for each facet and domain. The average scores reduce the overall score as well as the scores for each domain to a 4-point scale, which allows the clinician/physician to think of the individual's personality dysfunction relative to observed norms. The average facet score is calculated by dividing the raw facet score by the number of items in the facet (e.g., if all the items within the “Anhedonia” facet are rated as being “sometimes or somewhat true,” then the average facet score would be 16/8=2, indicating moderate anhedonia). An average domain score is calculated by summing and then averaging the 3 facet scores contributing primarily to the specific domain. For example, if the average facet scores on Anhedonia, Intimacy Avoidance and Withdrawal (scales primarily indexing Detachment) are all 2, then the sum of these scores would be 6, and the average domain score would be 6/3=2. Higher average scores indicate greater dysfunction in a specific personality trait facet or domain. High scores on a facet or domain may indicate significant and problematic areas for the individual receiving care that might warrant further assessment, treatment, and follow up.

Key aspects observed with patients/subjects suffering from psychomotor retardation are reduced energy and activity and reduced motivation. Psychomotor retardation involves a slowing down of thought and a reduction of physical movements in an individual. Psychomotor impairment may cause a visible slowing of physical and emotional reactions. Psychomotor retardation may be associated with one or more conditions or disorders, such as a mental disorder or a nervous system disorder or some other medical conditions.

Mental or nervous system disorders which lead to, or are associated with, psychomotor retardation include disorders characterised by depressive episodes, for example, Major Depressive Disorder (MDD); Bipolar Disorder (BD), such as Bipolar I Disorder and Bipolar II Disorder; Postpartum Depression (PPD); Seasonal Affective Disorder and Persistent Depressive Disorder; Mental and Behavioural Disorders due to Psychoactive Substance Use, for example Substance Use Disorder (SUD); Psychotic Disorder, for example Schizophrenia; Dementia, for example Alzheimer's Dementia (AD); Dementia with Lewy Bodies (DLB); Vascular Dementia and Parkinson's Disease Dementia; Parkinson's Disease; Chronic Fatigue Syndrome. Psychomotor retardation may also occur in a patient/subject suffering from sleep disturbance, for instance, insomnia.

Psychomotor retardation may be assessed by measuring various aspects. These may include for instance various types of drawing tasks and tests, such as the trail making test (TMT), the digit symbol substitution test (DSST), or the Gibson Spiral Maze Test (GSM) and others which are known in the art. In the trail making test (TMT), for instance, patients/subjects must connect 25 circles that contain either numbers (TMT A) or a combination of numbers and letters (TMT B) in ascending order. Task requirements are similar for TMT-B, except that the patient/subject must alternate between numbers and letters (1, A, 2, B, 3, C and so on). The test thus evaluates processing speed (TMT A) or cognitive flexibility (TMT B). The score for each part represents the amount of time required to complete the task.

Another test that involves graphomotor ability is the Gibson Spiral Maze (GSM) assessing psychomotor speed only, and is not influenced by cognitive abilities. patients/subjects who complete the GSM must correctly trace through a spiral maze from a starting point to an endpoint without touching bordering lines. The digit symbol substitution test (DSST) also measures psychomotor speed and consists of digit-symbol pairs followed by a list of digits. Under each digit, the patient/subject should write down the corresponding symbol as fast as possible. The score consists in the number of symbols correctly reported in 90 s. A further example for a motor test is the finger tapping test.

Thus, certain tests combine measurements of both motor and cognitive aspects of psychomotor retardation, while further others assess only motor aspects.

Analysis of speech may be a further indicator of psychomotor retardation. Major scales available to assess and measure include the severity of psychomotor retardation, the Salpetriere Retardation Rating Scale (SRRS) and the Motor Agitation and Retardation Scale (MARS). The Salpetriere Retardation Rating Scale (SRRS), developed by Widlocher assesses cognitive and motor aspects by fifteen items. The first three measure movement, specifically the quality of stride and slowness of limb, trunk, head, and neck movement. The next three items focus on speech including verbal flow, tone of voice, and length of response. Two items are designed to objectively measure cognitive function. These questions are based on the interview conversation and measure the patient's ability to approach and expand on topics. The further items are patient/subjective and assess rumination, fatigue, level of interest, perception of time, memory, and concentration. The last item of the scale relates to an overall assessment of the patient's psychomotor retardation. The items are scaled from 0 (symptom absence) to 4 (severe) based on the severity of the presenting symptom, for a total score range of 0 to 60.

The Motor Agitation and Retardation Scale (MARS) assesses motor aspects only. It was designed to assess psychomotor disturbances in depressive disorders. Psychomotor disturbances are divided into five major body categories including eyes, face, voice, limbs, and trunk with a total of 19 items on the scale. Items of the eyes category include direction of gaze, amount of blinking, staring, and eye movement. Items associated with the face category include facial expression and facial expressivity. The category of voice has items that include volume, slurring, tone and time for onset. Items under the limbs category include hand, foot, and leg movement, stride, motor slowness, and tension in hands. The trunk category items include posture, immobility, and axial movement. The severity of each item ranges from a 1 to a 4, with 4 being the most severe. Of the 19 items 9 relate to motor agitation and 10 items assess motor retardation. The retardation items include abnormal gait, immobility of trunk/proximal limbs, postural collapse, motor slowness (i.e. the limb and trunk category); lack of facial expressivity, downcast gaze (i.e. the eyes and face category); and reduced voice volume, slurring of speech, delayed speech onset, monotone speech (i.e. the voice category). The MARS scale offers a rapid clinical assessment of motor signs.

In an embodiment, the pharmaceutical composition of the invention is for use in the treatment of a patient with a AES-S score of at least 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or 72. In an embodiment, treatment of a patient with the pharmaceutical composition of the invention results in a decrease in AES-S score of at least 10, 20, 30, 40, 50, 60, 70, 80, 90 or 100%.

In an embodiment, treatment of a patient with the pharmaceutical composition of the invention results in a decrease in AES-S score of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or 72 points. In an embodiment, the pharmaceutical composition of the invention is for use in the treatment of a patient with a MADRS score of at least 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60. In an embodiment, treatment of a patient with the pharmaceutical composition of the invention results in a decrease in MADRS score of at least 10, 20, 30, 40, 50, 60, 70, 80, 90 or 100%. In an embodiment, treatment of a patient with the pharmaceutical composition of the invention results in a decrease in MADRS score of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 points. In an embodiment, the pharmaceutical composition of the invention is for use in the treatment of a patient with a GAD-7 score of at least 10, 11, 12, 13, 14, 15, 16, 17, 18, 19, 20 or 21 points. In an embodiment, treatment of a patient with the pharmaceutical composition of the invention results in a decrease in GAD-7 score of at least 10, 20, 30, 40, 50, 60, 70, 80, 90 or 100%. In an embodiment, treatment of a patient with the pharmaceutical composition of the invention results in a decrease in GAD-7 score of at least 2, 3, 4, 5, 6, 7, 8, 9, 10, 11, 12, 13, 14, 15, 16, 17, 18, 19, 20 or 21 points. In an embodiment, the pharmaceutical composition of the invention is for use in the treatment of a patient with a PHQ-9 score of at least 10, 11, 12, 13, 14, 15, 16, 17, 18, 19, 20, 21, 22, 23, 24, 25, 26, or 27 points. In an embodiment, treatment of a patient with the pharmaceutical composition of the invention results in a decrease in PHQ-9 score of at least 10, 20, 30, 40, 50, 60, 70, 80, 90 or 100%. In an embodiment, treatment of a patient with the pharmaceutical composition of the invention results in a decrease in PHQ-9 score of at least 2, 3, 4, 5, 6, 7, 8, 9, 10, 11, 12, 13, 14, 15, 16, 17, 18, 19, 20, 21, 22, 23, 24, 25, 26, or 27 points.

The skilled person will appreciate that references herein to pharmaceutically acceptable salts of 5-MeO-DMT include within their scope those salts, and crystalline forms thereof, as described herein.

### EMBODIMENTS

E. 1: A pharmaceutical composition comprising: (i) 5-methoxy-N,N-dimethyltryptamine (5-MeO-DMT) free base, or a pharmaceutically acceptable salt, prodrug, hydrate, ester, co-crystal or deuterated form thereof, and (ii) one or more pharmaceutically acceptable carriers or excipients, for use as a medicament in a method of treatment of one or more conditions or disorders in a patient in need thereof, said method comprising the administration of a first dose of the pharmaceutical composition to the patient followed by an administration of a second dose of the pharmaceutical composition to the patient, wherein the amount of 5-MeO-DMT in the second dose is higher than that in the first dose. E. 2: The pharmaceutical composition for use of embodiment 1, wherein the route of administration is intranasal. E. 3: The pharmaceutical composition for use of embodiment 2, wherein the first dose of the pharmaceutical composition is administered to a first nostril and the second dose of the pharmaceutical composition is administered to the other nostril. E. 4: The pharmaceutical composition for use of any one preceding embodiment, wherein the second dose of the pharmaceutical composition is administered within 30 minutes after the first dose. E. 5: The pharmaceutical composition for use of any one preceding embodiment, wherein the second dose of the pharmaceutical composition is administered within 20 minutes after the first dose. E. 6: The pharmaceutical composition for use of any one preceding embodiment, wherein the second dose of the pharmaceutical composition is administered within 15 minutes after the first dose. E. 7: The pharmaceutical composition for use of any one preceding embodiment, wherein the second dose of the pharmaceutical composition is administered within 10 minutes after the first dose. E. 8: The pharmaceutical composition for use of any one preceding embodiment, wherein the second dose of the pharmaceutical composition is administered within 5 minutes after the first dose. E. 9: The pharmaceutical composition for use of any one preceding embodiment, wherein the method comprises the administration of 1, 2, 3, 4, 5 or 6 mg 5-MeO-DMT as the first dose of 5-MeO-DMT. E. 10: The pharmaceutical composition for use of any one preceding embodiment, wherein the method comprises the administration of 4, 5, 6, 7, 8, 9, 10, 11 or 12 mg of 5-MeO-DMT as the second dose of 5-MeO-DMT. E. 11: The pharmaceutical composition for use of any one preceding embodiment, wherein the method comprises the administration of 4 mg 5-MeO-DMT as the first dose of 5-MeO-DMT. E. 12: The pharmaceutical composition for use of any one preceding embodiment, wherein the method comprises the administration of 8 mg of 5-MeO-DMT as the second dose of 5-MeO-DMT. E. 13: The pharmaceutical composition for use of any one preceding embodiment, wherein the method comprises the administration of 4 mg 5-MeO-DMT as the first dose of 5-MeO-DMT and the administration of 8 mg of 5-MeO-DMT as the second dose of 5-MeO-DMT. E. 14: The pharmaceutical composition for use of any one preceding embodiment, wherein the pharmaceutical composition is a dry powder. E. 15: The pharmaceutical composition for use of any one preceding embodiment, wherein the pharmaceutical composition comprises a mucoadhesive, optionally hydroxypropyl methyl cellulose (HPMC). E. 16: The pharmaceutical composition for use of any one preceding embodiment, wherein the anion of 5-MeO-DMT is selected from benzoate, hydrobromide, hydrochloride, phosphate, fumarate, oxalate, tartrate, benzenesulfonate, tosylate, glycolate, ketoglutarate, malate, saccharinate or succinate. E. 17: The pharmaceutical composition for use of any one preceding embodiment, wherein the 5-MeO-DMT salt is amorphous. E. 18: The pharmaceutical composition for use of any one preceding embodiment, wherein the 5-MeO-DMT salt is crystalline. E. 19: The pharmaceutical composition for use of any one preceding embodiment, wherein the 5-MeO-DMT salt is crystalline 5-MeO-DMT benzoate. E. 20: The pharmaceutical composition for use of any one preceding embodiment, wherein the 5-MeO-DMT salt is crystalline 5-MeO-DMT benzoate as characterised by one or more peaks in an X-ray powder diffraction (XRPD) diffractogram at a 2θ value of 17.5°±0.1°, 17.7°±0.1° and 21.0°±0.1° using an X-ray wavelength of 1.5406 Å. E. 21: The pharmaceutical composition for use of any one preceding embodiment, wherein the pharmaceutical composition is for use as a medicament in a method of treatment of one or more conditions or disorders in a patient in need thereof, wherein the use of the pharmaceutical composition causes a complete mystical experience to treat the one or more conditions in a patient in need thereof. E. 22: The pharmaceutical composition for use of any one preceding embodiment, wherein the pharmaceutical composition is for use as a medicament in a method of treatment of one or more conditions or disorders in a patient in need thereof, said conditions or disorders being selected from one or more of: treatment resistant depression, major depressive disorder, depression, persistent depressive disorder, alcohol use disorder, anxiety disorder, post-traumatic stress disorder (PTSD), body dysmorphic disorder, obsessive-compulsive disorder, eating disorder, psychoactive substance abuse and/or substance abuse. E. 23: The pharmaceutical composition for use of any one preceding embodiment, wherein the pharmaceutical composition is for use as a medicament in a method of treatment of treatment resistant depression, in a patient in need thereof. E. 24: The pharmaceutical composition for use of any one preceding embodiment, wherein the pharmaceutical composition is for use as a medicament in a method of treatment of major depressive disorder, in a patient in need thereof. E. 25: The pharmaceutical composition for use of any one preceding embodiment, wherein the pharmaceutical composition is for use as a medicament in a method of treatment of depression in a patient in need thereof. E. 26: The pharmaceutical composition for use of any one preceding embodiment, wherein the pharmaceutical composition is for use as a medicament in a method of treatment of depression, in a patient in need thereof. E. 27: The pharmaceutical composition for use of any one preceding embodiment, wherein the pharmaceutical composition is for use as a medicament in a method of treatment of alcohol use disorder, in a patient in need thereof. E. 28: The pharmaceutical composition for use of embodiment 1, wherein: the route of administration is intranasal; the first dose of the pharmaceutical composition is administered to a first nostril and the second dose of the pharmaceutical composition is administered to the other nostril; the second dose of the pharmaceutical composition is administered within 15 minutes after the first dose; the method comprises the administration of 4 mg 5-MeO-DMT as the first dose of 5-MeO-DMT; the method comprises the administration of 8 mg of 5-MeO-DMT as the second dose of 5-MeO-DMT; the 5-MeO-DMT salt is the benzoate; the pharmaceutical composition is a dry powder; administration of the pharmaceutical composition as per the method causes a complete mystical experience in the patient; the method of treatment is a method of treatment of depression; and wherein administration of the 12 mg 5-MeO-DMT benzoate as per the method is better tolerated by the patient than administration of the 12 mg of 5-MeO-DMT benzoate as a single dose. E. 29: The pharmaceutical composition for use of embodiment 1, wherein: the route of administration is intranasal; the first dose of the pharmaceutical composition is administered to a first nostril and the second dose of the pharmaceutical composition is administered to the other nostril; the second dose of the pharmaceutical composition is administered within 15 minutes after the first dose; the method comprises the administration of 4 mg 5-MeO-DMT as the first dose of 5-MeO-DMT; the method comprises the administration of 8 mg of 5-MeO-DMT as the second dose of 5-MeO-DMT; the 5-MeO-DMT salt is the hydrobromide; the pharmaceutical composition is a dry powder; administration of the pharmaceutical composition as per the method causes a complete mystical experience in the patient; the method of treatment is a method of treatment of depression; and wherein administration of the 12 mg 5-MeO-DMT hydrobromide as per the method is better tolerated by the patient than administration of the 12 mg of 5-MeO-DMT hydrobromide as a single dose. E. 30: The pharmaceutical composition for use of embodiment 1, wherein: the route of administration is intranasal; the first dose of the pharmaceutical composition is administered to a first nostril and the second dose of the pharmaceutical composition is administered to the other nostril; the second dose of the pharmaceutical composition is administered within 15 minutes after the first dose; the method comprises the administration of 4 mg 5-MeO-DMT as the first dose of 5-MeO-DMT; the method comprises the administration of 8 mg of 5-MeO-DMT as the second dose of 5-MeO-DMT; the pharmaceutical composition is a dry powder; administration of the pharmaceutical composition as per the method causes a complete mystical experience in the patient; the method of treatment is a method of treatment of depression; and wherein administration of the 12 mg 5-MeO-DMT as per the method is better tolerated by the patient than administration of the 12 mg of 5-MeO-DMT as a single dose. E. 31: A kit, for use as a medicament in a method of treatment of one or more conditions or disorders in a patient in need thereof, comprising: a first pharmaceutical composition comprising (i) 5-methoxy-N,N-dimethyltryptamine (5-MeO-DMT) free base, or a pharmaceutically acceptable salt, prodrug, hydrate, ester, co-crystal or deuterated form thereof; and (ii) one or more pharmaceutically acceptable carriers or excipients; a second pharmaceutical composition comprising (i) 5-methoxy-N,N-dimethyltryptamine (5-MeO-DMT) free base, or a pharmaceutically acceptable salt, prodrug, hydrate, ester, co-crystal or deuterated form thereof, and (ii) one or more pharmaceutically acceptable carriers or excipients; and optionally, instructions for use; wherein, the dose of 5-MeO-DMT in the second pharmaceutical composition is higher than the dose of 5-MeO-DMT in the first pharmaceutical composition. E. 32: The kit for use of embodiment 31, wherein the route of administration is intranasal. E. 33: The kit for use of embodiment 32, wherein the first dose of the pharmaceutical composition is administered to a first nostril and the second dose of the pharmaceutical composition is administered to the other nostril. E. 34: The kit for use of any one of embodiments 31 to 33, wherein the second dose of the pharmaceutical composition is administered within 30 minutes after the first dose. E. 35: The kit for use of any one of embodiments 31 to 34, wherein the second dose of the pharmaceutical composition is administered within 20 minutes after the first dose. E. 36: The kit for use of any one of embodiments 31 to 35, wherein the second dose of the pharmaceutical composition is administered within 15 minutes after the first dose. E. 37: The kit for use of any one of embodiments 31 to 36, wherein the second dose of the pharmaceutical composition is administered within 10 minutes after the first dose. E. 38: The kit for use of any one of embodiments 31 to 37, wherein the second dose of the pharmaceutical composition is administered within 5 minutes after the first dose. E. 39: The kit for use of any one of embodiments 31 to 38, wherein the method comprises the administration of 1, 2, 3, 4, 5 or 6 mg 5-MeO-DMT as the first dose of 5-MeO-DMT. E. 40: The kit for use of any one of embodiments 31 to 39, wherein the method comprises the administration of 4, 5, 6, 7, 8, 9, 10, 11 or 12 mg of 5-MeO-DMT as the second dose of 5-MeO-DMT. E. 41: The kit for use of any one of embodiments 31 to 40, wherein the method comprises the administration of 4 mg 5-MeO-DMT as the first dose of 5-MeO-DMT. E. 42: The kit for use of any one of embodiments 31 to 41, wherein the method comprises the administration of 8 mg of 5-MeO-DMT as the second dose of 5-MeO-DMT. E. 43: The kit for use of any one of embodiments 31 to 42, wherein the method comprises the administration of 4 mg 5-MeO-DMT as the first dose of 5-MeO-DMT and the administration of 8 mg of 5-MeO-DMT as the second dose of 5-MeO-DMT. E. 44: The kit for use of any one of embodiments 31 to 43, wherein the pharmaceutical composition is a dry powder. E. 45: The kit for use of any one of embodiments 31 to 44, wherein the pharmaceutical composition comprises a mucoadhesive, optionally hydroxypropyl methyl cellulose (HPMC). E. 46: The kit for use of any one of embodiments 31 to 45, wherein the anion of 5-MeO-DMT is selected from benzoate, hydrobromide, hydrochloride, phosphate, fumarate, oxalate, tartrate, benzenesulfonate, tosylate, glycolate, ketoglutarate, malate, saccharinate or succinate. E. 47: The kit for use of any one of embodiments 31 to 46, wherein the 5-MeO-DMT salt is amorphous. E. 48: The kit for use of any one of embodiments 31 to 47, wherein the 5-MeO-DMT salt is crystalline. E. 49: The kit for use of any one of embodiments 31 to 48, wherein the 5-MeO-DMT salt is crystalline 5-MeO-DMT benzoate. E. 50: The kit for use of any one of embodiments 31 to 49, wherein the 5-MeO-DMT salt is crystalline 5-MeO-DMT benzoate as characterised by one or more peaks in an X-ray powder diffraction (XRPD) diffractogram at a 2θ value of 17.5°±0.1°, 17.7°±0.1° and 21.0°±0.1° using an X-ray wavelength of 1.5406 Å. E. 51: The kit for use of any one of embodiments 31 to 50, wherein the pharmaceutical composition is for use as a medicament in a method of treatment of one or more conditions or disorders in a patient in need thereof, wherein the use of the pharmaceutical composition causes a complete mystical experience to treat the one or more conditions in a patient in need thereof. E. 52: The kit for use of any one of embodiments 31 to 51, wherein the pharmaceutical composition is for use as a medicament in a method of treatment of one or more conditions or disorders in a patient in need thereof, said conditions or disorders being selected from one or more of: treatment resistant depression, major depressive disorder, depression, persistent depressive disorder, alcohol use disorder, anxiety disorder, post-traumatic stress disorder (PTSD), body dysmorphic disorder, obsessive-compulsive disorder, eating disorder, psychoactive substance abuse and/or substance abuse. E. 53: The kit for use of any one of embodiments 31 to 52, wherein the pharmaceutical composition is for use as a medicament in a method of treatment of treatment resistant depression, in a patient in need thereof. E. 54: The kit for use of any one of embodiments 31 to 53, wherein the pharmaceutical composition is for use as a medicament in a method of treatment of major depressive disorder, in a patient in need thereof. E. 55: The kit for use of any one of embodiments 31 to 54, wherein the pharmaceutical composition is for use as a medicament in a method of treatment of depression in a patient in need thereof. E. 56: The kit for use of any one of embodiments 31 to 55, wherein the pharmaceutical composition is for use as a medicament in a method of treatment of depression, in a patient in need thereof. E. 57: The kit for use of any one of embodiments 31 to 56, wherein the pharmaceutical composition is for use as a medicament in a method of treatment of alcohol use disorder, in a patient in need thereof. E. 58: The kit for use of embodiment 31, wherein: the route of administration is intranasal; the first dose of the pharmaceutical composition is administered to a first nostril and the second dose of the pharmaceutical composition is administered to the other nostril; the second dose of the pharmaceutical composition is administered within 15 minutes after the first dose; the method comprises the administration of 4 mg 5-MeO-DMT as the first dose of 5-MeO-DMT; the method comprises the administration of 8 mg of 5-MeO-DMT as the second dose of 5-MeO-DMT; the 5-MeO-DMT salt is the benzoate; the pharmaceutical composition is a dry powder; administration of the pharmaceutical composition as per the method causes a complete mystical experience in the patient; the method of treatment is a method of treatment of depression; and wherein administration of the 12 mg 5-MeO-DMT benzoate as per the method is better tolerated by the patient than administration of the 12 mg of 5-MeO-DMT benzoate as a single dose. E. 59: The kit for use of embodiment 31, wherein: the route of administration is intranasal; the first dose of the pharmaceutical composition is administered to a first nostril and the second dose of the pharmaceutical composition is administered to the other nostril; the second dose of the pharmaceutical composition is administered within 15 minutes after the first dose; the method comprises the administration of 4 mg 5-MeO-DMT as the first dose of 5-MeO-DMT; the method comprises the administration of 8 mg of 5-MeO-DMT as the second dose of 5-MeO-DMT; the 5-MeO-DMT salt is the hydrobromide; the pharmaceutical composition is a dry powder; administration of the pharmaceutical composition as per the method causes a complete mystical experience in the patient; the method of treatment is a method of treatment of depression; and wherein administration of the 12 mg 5-MeO-DMT hydrobromide as per the method is better tolerated by the patient than administration of the 12 mg of 5-MeO-DMT hydrobromide as a single dose. E. 60: The kit for use of embodiment 31, wherein: the route of administration is intranasal; the first dose of the pharmaceutical composition is administered to a first nostril and the second dose of the pharmaceutical composition is administered to the other nostril; the second dose of the pharmaceutical composition is administered within 15 minutes after the first dose; the method comprises the administration of 4 mg 5-MeO-DMT as the first dose of 5-MeO-DMT; the method comprises the administration of 8 mg of 5-MeO-DMT as the second dose of 5-MeO-DMT; the pharmaceutical composition is a dry powder; administration of the pharmaceutical composition as per the method causes a complete mystical experience in the patient; the method of treatment is a method of treatment of depression; and wherein administration of the 12 mg 5-MeO-DMT as per the method is better tolerated by the patient than administration of the 12 mg of 5-MeO-DMT as a single dose. E. 61: A kit, for use as a medicament in a method of treatment of one or more conditions or disorders in a patient in need thereof, comprising: a first intranasal delivery device comprising a pharmaceutical composition comprising (i) 5-methoxy-N,N-dimethyltryptamine (5-MeO-DMT) free base, or a pharmaceutically acceptable salt, prodrug, hydrate, ester, co-crystal or deuterated form thereof, and (ii) one or more pharmaceutically acceptable carriers or excipients; a second intranasal delivery device comprising a pharmaceutical composition comprising (i) 5-methoxy-N,N-dimethyltryptamine (5-MeO-DMT) free base, or a pharmaceutically acceptable salt, prodrug, hydrate, ester, co-crystal or deuterated form thereof, and (ii) one or more pharmaceutically acceptable carriers or excipients; and optionally, instructions for use; wherein, the dose of 5-MeO-DMT in the pharmaceutical composition of the second intranasal delivery device is higher than the dose of 5-MeO-DMT in the pharmaceutical composition of the first intranasal delivery device. E. 62: The kit for use of embodiment 61, wherein the first dose of the pharmaceutical composition is administered to a first nostril and the second dose of the pharmaceutical composition is administered to the other nostril. E. 63: The kit for use of any one of embodiments 61 or 62, wherein the second dose of the pharmaceutical composition is administered within 30 minutes after the first dose. E. 64: The kit for use of any one of embodiments 61 to 63, wherein the second dose of the pharmaceutical composition is administered within 20 minutes after the first dose. E. 65: The kit for use of any one of embodiments 61 to 64, wherein the second dose of the pharmaceutical composition is administered within 15 minutes after the first dose. E. 66: The kit for use of any one of embodiments 61 to 65, wherein the second dose of the pharmaceutical composition is administered within 10 minutes after the first dose. E. 67: The kit for use of any one of embodiments 61 to 66, wherein the second dose of the pharmaceutical composition is administered within 5 minutes after the first dose. E. 68: The kit for use of any one of embodiments 61 to 67, wherein the method comprises the administration of 1, 2, 3, 4, 5 or 6 mg 5-MeO-DMT as the first dose of 5-MeO-DMT. E. 69: The kit for use of any one of embodiments 61 to 68, wherein the method comprises the administration of 4, 5, 6, 7, 8, 9, 10, 11 or 12 mg of 5-MeO-DMT as the second dose of 5-MeO-DMT. E. 70: The kit for use of any one of embodiments 61 to 69, wherein the method comprises the administration of 4 mg 5-MeO-DMT as the first dose of 5-MeO-DMT. E. 71: The kit for use of any one of embodiments 61 to 70, wherein the method comprises the administration of 8 mg of 5-MeO-DMT as the second dose of 5-MeO-DMT. E. 72: The kit for use of any one of embodiments 61 to 71, wherein the method comprises the administration of 4 mg 5-MeO-DMT as the first dose of 5-MeO-DMT and the administration of 8 mg of 5-MeO-DMT as the second dose of 5-MeO-DMT. E. 73: The kit for use of any one of embodiments 61 to 72, wherein the pharmaceutical composition is a dry powder. E. 74: The kit for use of any one of embodiments 61 to 73, wherein the pharmaceutical composition comprises a mucoadhesive, optionally hydroxypropyl methyl cellulose (HPMC). E. 75: The kit for use of any one of embodiments 61 to 74, wherein the anion of 5-MeO-DMT is selected from benzoate, hydrobromide, hydrochloride, phosphate, fumarate, oxalate, tartrate, benzenesulfonate, tosylate, glycolate, ketoglutarate, malate, saccharinate or succinate. E. 76: The kit for use of any one of embodiments 61 to 75, wherein the 5-MeO-DMT salt is amorphous. E. 77: The kit for use of any one of embodiments 61 to 76, wherein the 5-MeO-DMT salt is crystalline. E. 78: The kit for use of any one of embodiments 61 to 77, wherein the 5-MeO-DMT salt is crystalline 5-MeO-DMT benzoate. E. 79: The kit for use of any one of embodiments 61 to 78, wherein the 5-MeO-DMT salt is crystalline 5-MeO-DMT benzoate as characterised by one or more peaks in an X-ray powder diffraction (XRPD) diffractogram at a 2θ value of 17.5°±0.1°, 17.7°±0.1° and 21.0°±0.1° using an X-ray wavelength of 1.5406 Å. E. 80: The kit for use of any one of embodiments 61 to 79, wherein the pharmaceutical composition is for use as a medicament in a method of treatment of one or more conditions or disorders in a patient in need thereof, wherein the use of the pharmaceutical composition causes a complete mystical experience to treat the one or more conditions in a patient in need thereof. E. 81: The kit for use of any one of embodiments 61 to 80, wherein the pharmaceutical composition is for use as a medicament in a method of treatment of one or more conditions or disorders in a patient in need thereof, said conditions or disorders being selected from one or more of: treatment resistant depression, major depressive disorder, depression, persistent depressive disorder, alcohol use disorder, anxiety disorder, post-traumatic stress disorder (PTSD), body dysmorphic disorder, obsessive-compulsive disorder, eating disorder, psychoactive substance abuse and/or substance abuse. E. 82: The kit for use of any one of embodiments 61 to 81, wherein the pharmaceutical composition is for use as a medicament in a method of treatment of treatment resistant depression, in a patient in need thereof. E. 83: The kit for use of any one of embodiments 61 to 82, wherein the pharmaceutical composition is for use as a medicament in a method of treatment of major depressive disorder, in a patient in need thereof. E. 84: The kit for use of any one of embodiments 61 to 83, wherein the pharmaceutical composition is for use as a medicament in a method of treatment of depression in a patient in need thereof. E. 85: The kit for use of any one of embodiments 61 to 84, wherein the pharmaceutical composition is for use as a medicament in a method of treatment of depression, in a patient in need thereof. E. 86: The kit for use of any one of embodiments 61 to 85, wherein the pharmaceutical composition is for use as a medicament in a method of treatment of alcohol use disorder, in a patient in need thereof. E. 87: The kit for use of embodiment 61, wherein: the route of administration is intranasal; the first dose of the pharmaceutical composition is administered to a first nostril and the second dose of the pharmaceutical composition is administered to the other nostril; the second dose of the pharmaceutical composition is administered within 15 minutes after the first dose; the method comprises the administration of 4 mg 5-MeO-DMT as the first dose of 5-MeO-DMT; the method comprises the administration of 8 mg of 5-MeO-DMT as the second dose of 5-MeO-DMT; the 5-MeO-DMT salt is the benzoate; the pharmaceutical composition is a dry powder; administration of the pharmaceutical composition as per the method causes a complete mystical experience in the patient; the method of treatment is a method of treatment of depression; and wherein administration of the 12 mg 5-MeO-DMT benzoate as per the method is better tolerated by the patient than administration of the 12 mg of 5-MeO-DMT benzoate as a single dose. E. 88: The kit for use of embodiment 61, wherein: the route of administration is intranasal; the first dose of the pharmaceutical composition is administered to a first nostril and the second dose of the pharmaceutical composition is administered to the other nostril; the second dose of the pharmaceutical composition is administered within 15 minutes after the first dose; the method comprises the administration of 4 mg 5-MeO-DMT as the first dose of 5-MeO-DMT; the method comprises the administration of 8 mg of 5-MeO-DMT as the second dose of 5-MeO-DMT; the 5-MeO-DMT salt is the hydrobromide; the pharmaceutical composition is a dry powder; administration of the pharmaceutical composition as per the method causes a complete mystical experience in the patient; the method of treatment is a method of treatment of depression; and wherein administration of the 12 mg 5-MeO-DMT hydrobromide as per the method is better tolerated by the patient than administration of the 12 mg of 5-MeO-DMT hydrobromide as a single dose. E. 89: The kit for use of embodiment 61, wherein: the route of administration is intranasal; the first dose of the pharmaceutical composition is administered to a first nostril and the second dose of the pharmaceutical composition is administered to the other nostril; the second dose of the pharmaceutical composition is administered within 15 minutes after the first dose; the method comprises the administration of 4 mg 5-MeO-DMT as the first dose of 5-MeO-DMT; the method comprises the administration of 8 mg of 5-MeO-DMT as the second dose of 5-MeO-DMT; the pharmaceutical composition is a dry powder; administration of the pharmaceutical composition as per the method causes a complete mystical experience in the patient; the method of treatment is a method of treatment of depression; and wherein administration of the 12 mg 5-MeO-DMT as per the method is better tolerated by the patient than administration of the 12 mg of 5-MeO-DMT as a single dose. E. 90: A method of treatment of one or more conditions or disorders in a patient in need thereof, wherein the method comprises the administration of a first dose of 5-MeO-DMT free base, or a pharmaceutically acceptable salt, prodrug, hydrate, ester, co-crystal or deuterated form thereof, or a pharmaceutical composition thereof, followed by the administration of a second dose of 5-MeO-DMT free base, or a pharmaceutically acceptable salt, prodrug, hydrate, ester, co-crystal or deuterated form thereof, or a pharmaceutical composition thereof, wherein the second dose of 5-MeO-DMT is higher than the first dose. E. 91: The method of embodiment 90, wherein method of treatment comprises the occasioning of a complete mystical experience by the administration of the 5-MeO-DMT or a pharmaceutical composition thereof. E. 92: The method of embodiment 90 or embodiment 91, wherein the route of administration is intranasal. E. 93: The method of any one of embodiments 90 to 92, wherein the first dose of 5-MeO-DMT, or a pharmaceutical composition thereof, is administered to a first nostril and the second dose of 5-MeO-DMT, or a pharmaceutical composition thereof, is administered to the other nostril. E. 94: The method of any one of embodiments 90 to 93, wherein the second dose of 5-MeO-DMT, or a pharmaceutical composition thereof, is administered within 30 minutes after the first dose. E. 95: The method of any one of embodiments 90 to 94, wherein the second dose of 5-MeO-DMT, or a pharmaceutical composition thereof, is administered within 20 minutes after the first dose. E. 96: The method of any one of embodiments 90 to 95, wherein the second dose of 5-MeO-DMT, or a pharmaceutical composition thereof, is administered within 15 minutes after the first dose. E. 97: The method of any one of embodiments 90 to 96, wherein the second dose of 5-MeO-DMT, or a pharmaceutical composition thereof, is administered within 10 minutes after the first dose. E. 98: The method of any one of embodiments 90 to 97, wherein the second dose of 5-MeO-DMT, or a pharmaceutical composition thereof, is administered within 5 minutes after the first dose. E. 99: The method of any one of embodiments 90 to 98, wherein the method comprises the administration of 1, 2, 3, 4, 5 or 6 mg 5-MeO-DMT as the first dose of 5-MeO-DMT, or a pharmaceutical composition thereof. E. 100: The method of any one of embodiments 90 to 99, wherein the method comprises the administration of 4, 5, 6, 7, 8, 9, 10, 11 or 12 mg of 5-MeO-DMT, or a pharmaceutical composition thereof. E. 101: The method of any one of embodiments 90 to 100, wherein the method comprises the administration of 4 mg 5-MeO-DMT, or a pharmaceutical composition thereof, as the first dose of 5-MeO-DMT, or a pharmaceutical composition thereof. E. 102: The method of any one of embodiments 90 to 101, wherein the method comprises the administration of 8 mg of 5-MeO-DMT, or a pharmaceutical composition thereof, as the second dose of 5-MeO-DMT, or a pharmaceutical composition thereof. E. 103: The method of any one of embodiments 90 to 102, wherein the method comprises the administration of 4 mg 5-MeO-DMT, or a pharmaceutical composition thereof, as the first dose of 5-MeO-DMT, or a pharmaceutical composition thereof, and the administration of 8 mg of 5-MeO-DMT, or a pharmaceutical composition thereof, as the second dose of 5-MeO-DMT, or a pharmaceutical composition thereof. E. 104: The method of any one of embodiments 90 to 103, wherein the pharmaceutical composition is a dry powder. E. 105: The method of any one of embodiments 90 to 104, wherein the pharmaceutical composition comprises a mucoadhesive, optionally hydroxypropyl methyl cellulose (HPMC). E. 106: The method of any one of embodiments 90 to 105, wherein the anion of 5-MeO-DMT is selected from benzoate, hydrobromide, hydrochloride, phosphate, fumarate, oxalate, tartrate, benzenesulfonate, tosylate, glycolate, ketoglutarate, malate, saccharinate or succinate. E. 107: The method of any one of embodiments 90 to 106, wherein the 5-MeO-DMT salt is amorphous. E. 108: The method of any one of embodiments 90 to 107, wherein the 5-MeO-DMT salt is crystalline. E. 109: The method of any one of embodiments 90 to 108, wherein the 5-MeO-DMT salt is crystalline 5-MeO-DMT benzoate. E. 110: The method of any one of embodiments 90 to 109, wherein the 5-MeO-DMT salt is crystalline 5-MeO-DMT benzoate as characterised by one or more peaks in an X-ray powder diffraction (XRPD) diffractogram at a 2θ value of 17.5°±0.1°, 17.7°±0.1° and 21.0°±0.1° using an X-ray wavelength of 1.5406 Å. E. 111: The method of any one of embodiments 90 to 110, wherein the method of treatment of one or more conditions or disorders in a patient in need thereof is selected from one or more of: treatment resistant depression, major depressive disorder, depression, persistent depressive disorder, alcohol use disorder, anxiety disorder, post-traumatic stress disorder (PTSD), body dysmorphic disorder, obsessive-compulsive disorder, eating disorder, psychoactive substance abuse and/or substance abuse. E. 112: The method of any one of embodiments 90 to 111, wherein the method of treatment is a method of treatment of treatment resistant depression, in a patient in need thereof. E. 113: The method of any one of embodiments 90 to 111, wherein the method of treatment is a method of treatment of major depressive disorder, in a patient in need thereof. E. 114: The method of any one of embodiments 90 to 111, wherein the method of treatment is a method of treatment of depression in a patient in need thereof. E. 115: The method of any one of embodiments 90 to 111, wherein the method of treatment is a method of treatment of alcohol use disorder, in a patient in need thereof. E. 116: The method of treatment of embodiment 90, wherein: the route of administration is intranasal; the first dose of 5-MeO-DMT, or a pharmaceutical composition thereof, is administered to a first nostril and the second dose of 5-MeO-DMT, or a pharmaceutical composition thereof, is administered to the other nostril; the second dose of 5-MeO-DMT, or a pharmaceutical composition thereof, is administered within 15 minutes after the first dose; the method comprises the administration of 4 mg 5-MeO-DMT, or a pharmaceutical composition thereof, as the first dose of 5-MeO-DMT; the method comprises the administration of 8 mg of 5-MeO-DMT, or a pharmaceutical composition thereof, as the second dose of 5-MeO-DMT; the 5-MeO-DMT salt is the benzoate; administration of 5-MeO-DMT, or a pharmaceutical composition thereof, as per the method causes a complete mystical experience in the patient; the method of treatment is a method of treatment of depression; and wherein administration of the 12 mg 5-MeO-DMT benzoate as per the method is better tolerated by the patient than administration of the 12 mg of 5-MeO-DMT benzoate as a single dose. E. 117: The method of treatment of embodiment 90, wherein: the route of administration is intranasal; the first dose of 5-MeO-DMT, or a pharmaceutical composition thereof, is administered to a first nostril and the second dose of 5-MeO-DMT, or a pharmaceutical composition thereof, is administered to the other nostril; the second dose of 5-MeO-DMT, or a pharmaceutical composition thereof, is administered within 15 minutes after the first dose; the method comprises the administration of 4 mg 5-MeO-DMT, or a pharmaceutical composition thereof, as the first dose of 5-MeO-DMT; the method comprises the administration of 8 mg of 5-MeO-DMT, or a pharmaceutical composition thereof, as the second dose of 5-MeO-DMT; the 5-MeO-DMT salt is the hydrobromide; administration of 5-MeO-DMT, or a pharmaceutical composition thereof, as per the method causes a complete mystical experience in the patient; the method of treatment is a method of treatment of depression; and wherein administration of the 12 mg 5-MeO-DMT hydrobromide as per the method is better tolerated by the patient than administration of the 12 mg of 5-MeO-DMT hydrobromide as a single dose. E. 118: The pharmaceutical composition for use of any one of embodiments 1 to 30, the kit for use of any one of embodiments 31 to 89 or the method of any one of embodiments 90 to 117, wherein the method comprises the administration of 5-MeO-DMT, or a pharmaceutical composition thereof, to the patient by the patient. E. 119: The pharmaceutical composition for use of any one of embodiments 1 to 30, the kit for use of any one of embodiments 31 to 89 or the method of any one of embodiments 90 to 117, wherein the method comprises the administration of 5-MeO-DMT, or a pharmaceutical composition thereof, to the patient by a medical professional. E. 120: The pharmaceutical composition for use of any one of embodiments 1 to 30, the kit for use of any one of embodiments 31 to 89 or the method of any one of embodiments 90 to 117, wherein the method of treatment is a method of treatment of one or more conditions or disorders wherein the one or more conditions or disorders have been clinically diagnosed in accordance with accepted medical practice. E. 121: The pharmaceutical composition for use of any one of embodiments 1 to 30 or 118 to 120, the kit for use of any one of embodiments 31 to 89 or the method of any one of embodiments 90 to 117 or 118 to 120, or the method of any one of embodiments 90 to 117 or 118 to 120, wherein administration of 5-MeO-DMT, or a pharmaceutical composition thereof, induces a complete mystical experience, optionally as identified through achievement of at least 60% of the maximum possible score in each of the four subscales (mystical, positive mood, transcendence of time and space, and ineffability) of the 30-claim revised Mystical Experience Questionnaire (MEQ30) and/or through achievement of at least 60% of the maximum possible score of the Oceanic Boundlessness (OBN) dimension of the Altered States of Consciousness (ASC) questionnaire, wherein optionally, the complete mystical experience is induced within 5, 10, 15, 20, 25, or 30 minutes of administration and wherein, optionally, the complete mystical experience is resolved within 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 minutes of administration. In an embodiment, the intranasal delivery device is a single use intranasal delivery device, optionally, an active delivery device, optionally an active delivery device which does not require the patient to inhale through the nose to deliver the pharmaceutical composition.

### BRIEF DESCRIPTION OF THE DRAWINGS

FIG. 1 shows the mean (+/−SD) 5-MeO-DMT HCl plasma log concentration-time plot.

FIG. 2 shows the mean (+/−SD) 5-MeO-DMT benzoate plasma linear concentration-time plot.

FIG. 3 shows the mean (+/−SD) 5-MeO-DMT benzoate plasma log concentration-time plot.

FIG. 4 shows a representation of MEQ30 scores.

FIG. 5 shows a representation of MEQ30 scores.

FIG. 6 shows a further representation of MEQ30 scores.

FIG. 7 shows a representation of the Subjective Dose Intensity (SDI) alongside the Pharmacokinetics (PKs) for a 12 mg monophasic dose (a single dose) of 5-MeO-DMT benzoate and a 12 mg biphasic dose (two doses administered apart, the doses together totalling 12 mg) of 5-MeO-DMT benzoate.

FIG. 8 shows predicted pharmacokinetics.

FIG. 9 shows the particle size distribution of a 5-MeO-DMT SDD as Bulk Material (Red) and ExDevice (Green).

FIG. 10 shows the nasal deposition profile for a 5-MeO-DMT SDD delivered via an active delivery nasal delivery device.

FIG. 11 shows the nasal deposition profile for a 5-MeO-DMT SDD delivered via a passive delivery nasal delivery device.

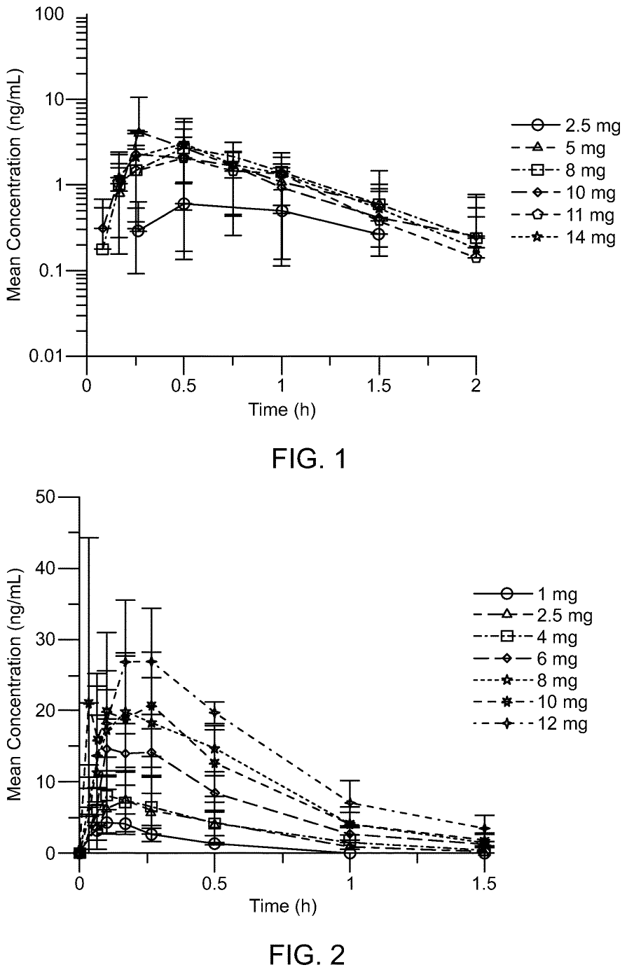

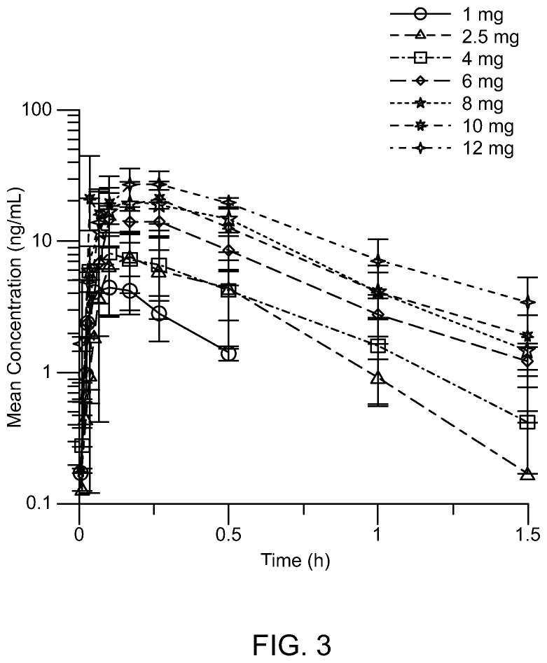

### EXAMPLES

### Example 1: Comparison of MEQ30 Scores

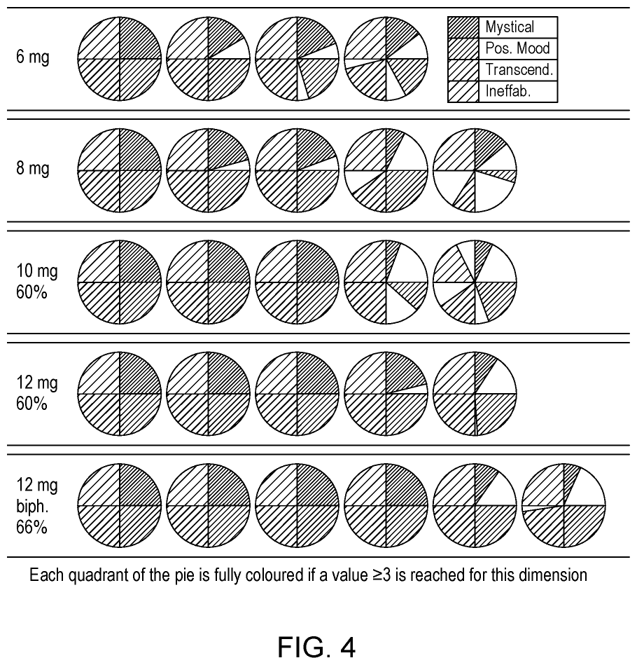

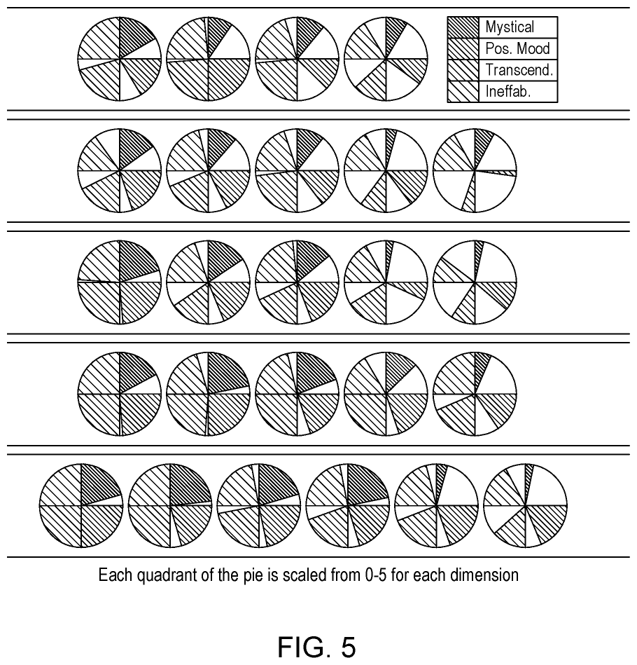

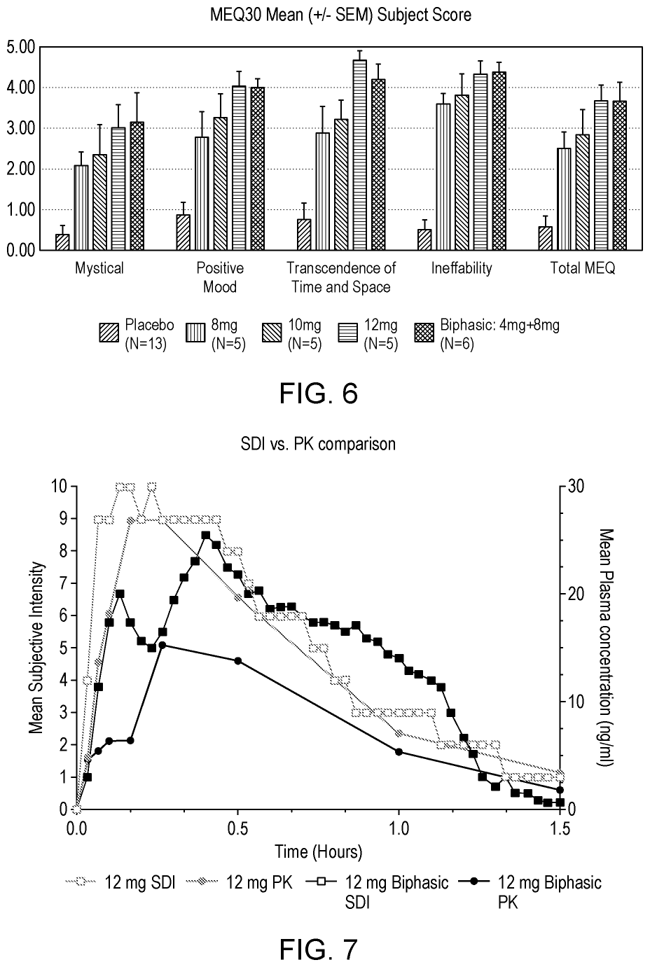

FIG. 4 shows a representation of the MEQ30 scores of individuals following: (1) a 6 mg intranasal dose of 5-MeO-DMT benzoate into a single nostril; (2) an 8 mg intranasal dose of 5-MeO-DMT benzoate into a single nostril; (3) a 10 mg intranasal dose of 5-MeO-DMT benzoate into a single nostril; (4) a 12 mg intranasal dose of 5-MeO-DMT benzoate into a single nostril; and (5) a total intranasal dose of 12 mg of 5-MeO-DMT benzoate administered in a biphasic fashion, i.e. an initial 4 mg dose into one nostril, followed by an 8 mg dose into the other nostril. Each quadrant of a pie is fully coloured if a value equal to or greater than 3 is reached for that dimension (wherein the four quadrants represent the four factors of the MEQ30: mystical, positive mood, transcendence of time and space, and ineffability). Each pie represents a single individual who received the respective dose. It can be seen that the 4/6 individuals who received 12 mg total dose of 5-MeO-DMT benzoate in a biphasic fashion achieved mystical experiences, which represents a 66% success rate. This is a higher success rate than that seen for those individuals who received a singular 12 mg dose or a singular 10 mg dose, both of which had a 60% success rate. FIG. 5 shows a further representation of the findings shown in FIG. 4, in this Figure each quadrant of a pie is scaled from 0-5 for each dimension. FIG. 6 shows a further representation of the MEQ30 scores reported by the subjects (including subjects who received placebo).

### Example 2: Subjective Dose Intensity, Pharmacokinetic Profiles

FIG. 7 shows a representation of the Subjective Dose Intensity (SDI) alongside the Pharmacokinetics (PKs) for a 12 mg monophasic dose of 5-MeO-DMT benzoate and a 12 mg biphasic dose of 5-MeO-DMT benzoate. It can be seen that there was a slower rise to peak mean plasma concentration following the biphasic dose alongside a lower peak plasma concentration and a longer psychedelic experience. The Table below shows results from the administration of (1) a 6 mg intranasal dose of 5-MeO-DMT benzoate into a single nostril; (2) an 8 mg intranasal dose of 5-MeO-DMT benzoate into a single nostril; (3) a 10 mg intranasal dose of 5-MeO-DMT benzoate into a single nostril; (4) a 12 mg intranasal dose of 5-MeO-DMT benzoate into a single nostril; and (5) a total intranasal dose of 12 mg of 5-MeO-DMT benzoate administered in a biphasic fashion, i.e. an initial 4 mg dose into one nostril, followed by an 8 mg dose into the other nostril.

| | | | | | | 12 mg |
|---|---|---|---|---|---|---|
| Dose | 4 mg | 6 mg | 8 mg | 10 mg | 12 mg | (biphasic) |
| Cmax (ng/ml) | 8.7 | 14.9 | 22.1 | 32.3 | 28.9 | 16.7 |
| Tmax (min.) | 7.0 | 9.0 | 10.6 | 8.4 | 15.3 | 27 |
| T½ (min) | 22.4 | 22.2 | 17.7 | 22.7 | 26.6 | 16.1 |
| AUC inf (h*ng/ml | 5.0 | 9.8 | 14 | 15.4 | 23 | 10.7 |
| Duration of the trip (estimate based on | 18 min | 31 min | 40 min | 46 min. | 50 min. | 70 min. |
| intensity) | | | | | | |
| Peak exp. (%) | 0 | 25% | 20% | 60% | 60% | 66% |
| Vomiting (% subj. incl. PBO ) | 33% | 0% | 0% | 0% | 29% | 17% |

It can be seen that administration of 12 mg in a biphasic fashion results in: (1) longer duration of ‘trip’; (2) the highest % peak experience; and (3) a lower incidence of vomiting compared with 12 mg monophasic (17% vs 29%).

### Example 3: Expected vs Observed Pharmacokinetics

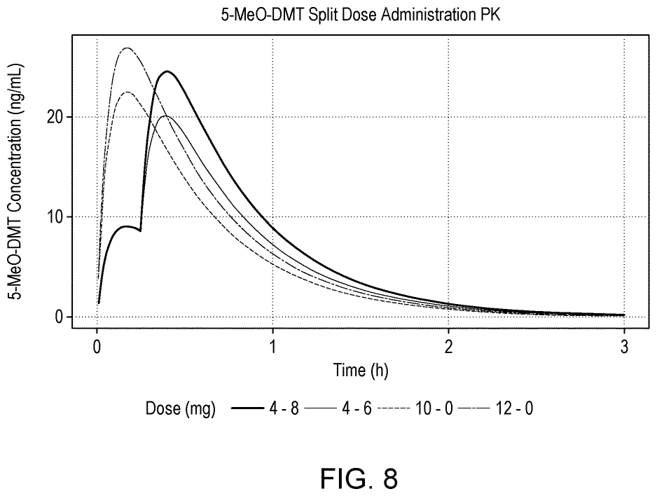

The biphasic dosing approach for 5-MeO-DMT benzoate may comprise the administration of a first dose followed 15 minutes later by a second dose which is higher than the first dose. Pharmacokinetic simulation of this dosing approach predicted a Tmax after 25-30 minutes with a Cmax which was predicted to be ~16% lower compared to the same total dose administered in a single dose. The predicted pharmacokinetics can be seen in FIG. 8. When FIGS. 7 and 8 are compared, it can be seen that the observed pharmacokinetics differ from those expected. The peak plasma concentration obtained from the biphasic administration is significantly lower than that which was expected. Despite this, as unexpectedly shown in the aforementioned Figures, this did not result in a lower subjective intensity/mystical experience in the subjects. Without wishing to be bound by theory, it may be understood that during monophasic administration the enzyme(s) involved with breaking down 5-MeO-DMT become saturated. During biphasic administration, the enzyme(s) do not become saturated to such an extent, explaining the difference in peak plasma concentration.

### Example 4: Medical Support Practitioners Therapists

Medical/support practitioners/therapists also approved the biphasic dosing as compared to the monophasic dosing of 5-MeO-DMT. The biphasic approach gives agency to the subject being treated, they have agency as to whether to proceed onwards to the second (higher) dose after receipt of the initial (lower) dose. Without wishing to be bound by theory, it is believed that this reduces anxiety in the subject and so lowers the overall risk of unwanted adverse/challenging experiences/psychiatric effects. Also, it is believed that the first dose in effect allows the subject to relax and so gives them confidence that they can handle the second higher dose. Additionally, a test subject who has never had a psychedelic experience before, is better able to gauge if they would like to proceed to the second (often) higher dose. In that way, medical consent is more informed in light of having the first dose. As such, an additional benefit provided by the invention is that medical/support practitioners/therapists approved the biphasic dosing over the monophasic dosing of 5-MeO-DMT. This means that the medical/support practitioners/therapists would be more inclined to adopt this approach when treating subjects and so this treatment is more likely to get to patients in need of such treatments.

### Example 5: Targeted Nasal Delivery

The nasal cavity is recognised as a promising systemic drug delivery route due to the highly vascularised capillary bed within the nasal mucosa. There is therefore a need for formulations or compositions as described herein with an optimised particle size distribution which show turbinate deposition. There is also a need for delivery devices which can selectively deliver a formulation or composition as described herein to the nasal turbinates, for the uses as described herein.

### Materials

5-methoxy-N,N-dimethyltryptamine (5-MeO-DMT) Benzoate, Hydroxypropyl methylcellulose (HPMC) ((Pharmacoat 606—substitution 2910, viscosity 6 cP) ShinEtsu Chemical, Japan), HPLC grade 99% ethanol, HPLC grade 99% methanol, HPLC grade water, Glycerol and Brij-35 (Fisher Scientific, United Kingdom). Ultrapure water 18.2 MQ (Veolia Elga LabWater system, in house) Active devices (UDSp, Aptar Pharma, France).

### Preparation of 5-MeO-DMT Spray Dried Dispersion (5-MeO-DMT SDD)

Feed solution was prepared at 50% w/w 5-MeO-DMT Benzoate loading (32.1% 5-MeO-DMT). Both polymers were dissolved in water under ambient stirring overnight. D-sorbitol and 5-MeO-DMT Benzoate were added to solution and dissolved under ambient stirring, producing a clear, lightly straw-coloured solution. Feed solution was spray dried using the ProCepT 4M8-Trix Spray Dryer fitted with a 25 kHz Ultrasonic nozzle (ProCepT, Belgium), according to the spray drying parameters outlined in the Table below. Formulation was filled and assembled into UDSp devices at 37.4±1.9 mg fill weight, under reduced humidity, when required for analysis.

### Target Spray Drying Parameters for 5-MeO-DMT Feed Solution

| Inlet | Outlet | Nozzle | Cooling | Drying | Liquid |
|---|---|---|---|---|---|
| temperature | temperature | air | air | airflow | feed rate |
| (° C.) | (° C.) | (bar) | (bar) | (m3/min) | (g/min) |
| 105 | 80-85 | 2.00 | 0.5 | 0.70 | 2.0 |

### Particle Size Analysis by Laser Diffraction

Particle Size Distribution (PSD) was determined using a Sympatec HELOS H4459 particle size analyser equipped with an R5 lens (Sympatec GmbH, Germany) in triplicate. Bulk powder was analysed using the RODOS dry powder dispersion unit at 3 bar dispersal pressure and powder from active devices (ExDevice) were manually actuated into the laser diffractor with the tip of the device positioned 3 cm from the mid-point of the laser.

### Methodology—Summary

The Alberta Idealised Nasal Inlet (AINI) and Stage 1 collection cup of the Next Generation Impactor (NGI) was coated with a solution containing 12 g Brij-35, 20 g glycerol and 80 mL ethanol. Once dried, AINI and NGI were assembled with the addition of a pre-separator with 15 mL 50:50 (v/v) methanol:water diluent in the reservoir. The UDSp loaded with 37.4 mg formulation was positioned at either 30, 45 or 60° to the horizontal and inserted 1 cm into the nasal orifice of the AINI. A 7.5 L/min airflow was applied for 15 seconds upon actuation of the UDSp, delivering 1.875 L of air. After actuation, the configuration was disassembled and 15 mL diluent used to dissolve material on the USps exterior, deposited in the AINI and the NGI collection cup, with the addition of a secondary dilution. Analysis was performed in triplicate and analysed by HPLC, with Two-way ANOVA statistical analysis.

### Methodology—Detailed

Nasal deposition was measured using the Alberta Idealised Nasal Inlet (AINI) with the Copley Next Generation Impactor (NGI) from an Aptar Unidose Powder Nasal spray system (UDSp)
A) Coating of AINI and NGI collection cups: 12 g Brij-35, 20 g glycerol and 80 mL ethanol were mixed until dissolved to form a coating solution. Bottom of the AINI was sealed and a 20 mL coating solution was added through the vestibule while the AINI was inverted. The AINI was slowly rotated horizontally 360° clockwise and anticlockwise then rotated vertically 360° clockwise and anticlockwise. Excess coating solution was drained and the AINI was placed on its left side, back and right side for 15 minutes each. AINI was positioned upright for 30 minutes to allow any further excess coating solution to drain and for the coat to dry. 2 mL of coating solution was pipetted onto the NGI Stage 1 collection cup and rocked for 5 minutes using the NGI rocker in order to coat. Excess solution was drained and the cup was allowed to dry.
B) NGI assembly: NGI was assembled with the coated Stage 1 collection cup and uncoated collection cups for states 2-7 and micro-orifice collector. The pre-separator and throat piece were attached and a leak test was performed using the critical flow controller and high-capacity pump. The flowmeter was attached to the throat piece and the flowrate set to 7.5 L/min. The throat piece was removed and 15 mL 50:50% v/v HPLC grade water: HPLC grade methanol (diluent) was added to the pre-separator insert cup. The AINI was then installed on the pre-separator.
C) Actuation: The UDSp containing 5-MeO-DMT was weighed to obtain a pre-actuation mass. The UDSp was then clamped into position such that the tip of the UDSp was inserted 1 cm into the vestibule of the AINI. The angle of insertion was set using an electronic protractor. The UDSp was actuated using 7.5 L/min flow rate for 15 seconds.
D) HPLC Sample collection: UDSp was then removed and weighed to obtain a post-actuation mass. The exterior of the UDSp was washed with 15 mL diluent in a glass dish and the washings were collected for HPLC analysis. AINI was disassembled and each component was thoroughly washed in separate glass dishes with 15 mL diluent. These washings were then collected for HPLC analysis. The pre-separator was removed from the NGI, the top and bottom were then covered and the pre-separator was inverted to wash the interior with the previously added 15 mL diluent. These washings were then collected for HPLC analysis. 15 mL diluent was added to the Stage 1 collection cup and the cup was rocked for 10 minutes using the NGI rocker to wash. These washings were then collected for HPLC analysis.
E) HPLC: HPLC was carried out on the samples to quantify 5-MeO-DMT content. Where necessary samples were diluted to stay within the linearity of the quantification method.

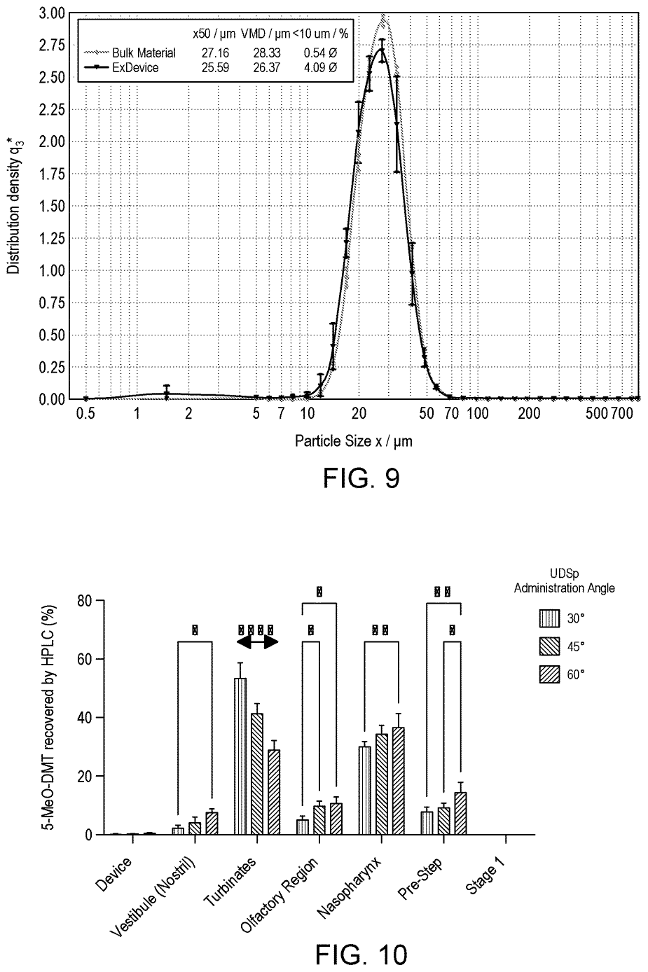

Results: Pharmacopeia guidelines state that a nasal powder should demonstrate that deposition of the products is localised within the nasal cavity, and the current method requires that most of the particles are larger than 10 μm as determined by laser diffraction. Analysis was performed on the 5-MeO-DMT SDD with the Sympatec, and the particle size distribution shown in FIG. 9. Using the ultrasonic nozzle an optimised nasal powder was achieved in which the mean particle diameter was close to the diameter recommended while maintaining minimal particles below 10 μm, passing acceptance criteria requested by the European Medicines Agency (EMA).

The AINI was used to assess the deposition profile of the 5-MeO-DMT SDD formulation with the method outlined above. The AINI consists of four components for the nasal cavity—the vestibule (nostril), turbinates, olfactory and nasopharynx—which is assembled and attached to a pre-separator. The pre-separator is incorporated to capture any deposition that would falsely land on Stage 1 due to particle bounce in the internal surfaces of the AINI. Minimal deposition was seen in the vestibule compared to commercially available nasal sprays. Minimal deposition was also seen in the lung analogue. The majority of the formulation was found in the turbinates and the olfactory region showed deposition from 5-11%—which is advantageous as it is theorised that a minimum of 0.01-1% of the oral dose is effective for nose-to-brain absorption. The results of the AINI can be seen in FIG. 10.

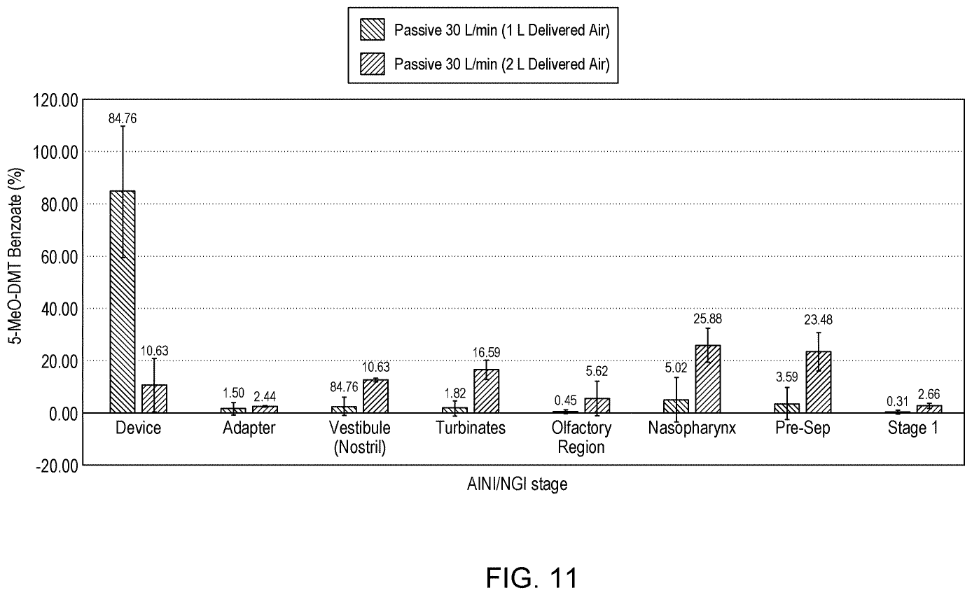

There is therefore provided an advantageous method for the delivery of a 5-MeO-DMT SDD. The same 5-MeO-DMT SDD was filled into the passive nasal delivery device at a loading suitable to deliver 12 mg 5-MeO-DMT free base equivalent. The passive device was positioned into an adapter and drawn through the AINI/NGI with a flow rate of 30 L/min to deliver either 1 L or 2 L of air respectively. The nasal deposition profile produced can be seen in FIG. 11. It can be readily seen that very little drug product was deposited in the desired locations of the turbinates and olfactory region.

There is therefore provided an advantageous method for the delivery of a 5-MeO-DMT wherein 5-MeO-DMT is delivered by an active nasal delivery device. In an embodiment, there is provided the use of a formulation or composition as described herein in a method of treating a patient in need thereof, wherein the formulation or composition is administered intranasally via an active delivery nasal device, as described herein, and wherein more than 30%, 40%, 50%, 60%, 70%, 80% or 90% of the formulation or composition is deposited to the turbinates and/or olfactory region of the nasal cavity. In an embodiment, the method of treating a patient in need thereof is a method of treating one or more of the conditions or diseases described herein.

In an embodiment, there is provided the use of a formulation or composition as described herein in a method of treating a patient in need thereof, wherein the formulation or composition is administered intranasally via an active delivery nasal device and wherein less than 30%, 25%, 20%, 15%, 10%, 5%, 4%, 3%, 2% or 1% is deposited in the lungs. In an embodiment, there is provided a nasal delivery device for delivering a formulation or composition as described herein to an olfactory region of a nasal cavity, the device comprising a formulation or composition as described herein. In an embodiment, the device is an active nasal delivery device wherein a plunger style actuator, or similar, is depressed to administer a dose. In an embodiment, the nasal delivery device is not a breath actuated delivery device. In an embodiment, the device comprises a dose volume up to 140 mm³.

In an embodiment the device is an Aptar device (UDS—Unidose Solid) as commercially available in the UK as of 1 Jun. 2023. In an embodiment, the dry powder is administered to the subject using a dry powder device as described in U.S. Publication 2016/0296957, which is hereby incorporated by reference in its entirety. Dry powder devices described in U.S. Publication No. 2022/0362491, International Publication No. WO 2022/123128, International Publication No. WO 2022/171969; and International Publication No. WO 2022/208014 are incorporated herein by reference.

In an embodiment, the nasal delivery device may be as described in any one of WO21005308; WO22123128; WO22171969; and WO22208014 (the contents of which are incorporated by reference).

In an embodiment, there is provided a dispenser device, optionally for dispensing a formulation or composition as described herein, the dispenser device comprising: a formulation or composition as described herein; a dispenser outlet (10); an air expeller (20) for generating a flow of air while the device is being actuated, said air expeller (20) including a piston (21) that slides in an air chamber (22) between a rest position and a dispensing position, said air chamber (22) including a cylindrical body (222) in which said piston (21) slides in airtight manner; and at least one reservoir (30) that contains a single dose of composition, said reservoir (30) including an air inlet (31) that is connected to said air expeller (20), and a composition outlet (32) that is connected to said dispenser outlet (10), said air inlet (31) including a composition retainer member (40) for retaining the composition in the reservoir (30) until the composition is dispensed, and said composition outlet (32) being closed by a closure element (50) that is force fitted in the composition outlet (32) of the reservoir (30); said device further including a mechanical opening system (61, 62) that co-operates with said closure element (50) so as to expel it mechanically from its closed position while the device is being actuated, said mechanical opening system comprising a rod assembly (61, 62), a first rod portion (61) being part of said air expeller (20) and sliding in said air chamber (22) during actuation of the device, and a second rod portion (62) pushed by said first rod portion (61) during actuation of the device, said rod assembly (61, 62) cooperating at the end of the actuation stroke with said closure element (50) to expel it mechanically from its closed portion, said piston (21) of said air expeller (20), when in its rest position, co-operating in non-airtight manner with said air chamber (22), in such a manner that said air chamber (22) is in communication with the atmosphere in the rest position, said piston (21) including an inner lip (215) that slides in airtight manner on said cylindrical surface (614) during actuation of the device, and that co-operates in non-airtight manner with fluting (615) formed on said cylindrical surface (614) in the rest position to put the air chamber (22) in communication with the atmosphere in the rest position, said piston 21) co-operating in airtight manner with said cylindrical body (222) in any positions, and co-operating in non-airtight manner with said cylindrical surface (614) only in the rest position.

In an embodiment, there is provided a dispenser device, optionally for dispensing a formulation or composition as described herein, the dispenser device comprising: a formulation or composition as described herein; a dispenser outlet; an air expeller for generating a flow of air while the device is being actuated, said air expeller including a piston that slides in an air chamber between a rest position and a dispensing position, said air chamber including a cylindrical body in which said piston slides in airtight manner; and at least one reservoir that contains a single dose of composition, said reservoir including an air inlet that is connected to said air expeller, and a composition outlet that is connected to said dispenser outlet, said air inlet including a composition retainer member for retaining the composition in the reservoir until the composition is dispensed, and said composition outlet being closed by a closure element that is force fitted in the composition outlet of the reservoir; said device further including a mechanical opening system that co-operates with said closure element so as to expel said closure element mechanically from a closed position while the device is being actuated, said piston of said air expeller, when in the rest position, co-operating in non-airtight manner with said air chamber, in such a manner that said air chamber is in communication with the atmosphere in the rest position, wherein said piston includes an inner lip configured to cooperate with a cylindrical surface of a cylindrical member extending inside the cylindrical body, said cylindrical surface including fluting that co-operates in non-airtight manner with said inner lip of the piston in the rest position.

In an embodiment, said piston comprises one or more markings which are visible only when the piston is in the rest position and not visible when the piston is in the dispensing position. In an embodiment, a method comprising the use of the dispenser device comprises the actuation of the piston from the rest position to the dispensing position such that the one or more markings are no longer visible. In an embodiment, successful actuation of the dispensing device occurs when the one or more markings are no longer visible. In an embodiment, the one or more markings may be: one or more coloured lines, one or more coloured shapes, one or more words or written text or one or more physical features.

In an embodiment, there is provided an active nasal delivery device as described herein comprising one or more markings, said markings being visible when the active nasal delivery device is in the resting state and not visible following successful actuation of said active nasal delivery device. In an embodiment, the absence from view of said markings represents successful actuation of the active nasal delivery device. In an embodiment, the one or more markings may be as described herein.

A method of intranasally delivering a powder pharmaceutical formulation comprising a psychedelic and one or more pharmaceutically acceptable carriers or excipients, to a patient, wherein 30% or more, 40% or more, 50% or more, 60% or more, 70% or more, 80% or more, 90% or more, 91% or more, 92% or more, 93% or more, 94% or more, 95% or more, 96% or more, 97% or more, 98% or more or 99% or more of the formulation reaches the turbinates and olfactory region, wherein the psychedelic is 5-MeO-DMT, or a pharmaceutically acceptable salt thereof, and the formulation is delivered via a nasal powder dispenser device which may comprise one or more of: a nasal dispenser head for inserting into a patient's nostril, the nasal dispenser head including a dispenser orifice; and an air expeller that, during actuation of the nasal powder dispenser device, generates a flow of compressed air so as to dispense a dose of the powder pharmaceutical formulation into the nostril through the dispenser orifice.

In an embodiment, there is provided an intranasal delivery system, optionally comprising an active nasal delivery device as described herein, comprising: a dry powder formulation comprising a plurality of powder particles of 5-MeO-DMT, or a pharmaceutically acceptable salt thereof, and an active nasal delivery device, optionally as described herein, configured to deliver the particles to the turbinates and olfactory region of the nasal cavity of a subject at a single actuation; wherein the system is operably configured to emit a powder plume comprising 5-MeO-DMT, or a pharmaceutically acceptable salt thereof, having one or more of:
a plume geometry of: angle: 20 to 45 degrees; width: 25 to 55 mm;
a spray pattern of:

| Distance 40 mm: | Distance 70 mm: |
|---|---|
| Dmin(mm): 15 to 25 | Dmin(mm): 15 to 40 |
| Dmax(mm): 20 to 55 | Dmax(mm): 35 to 60 |
| Area (mm²): 390 to 900 | Area (mm²): 800-1600 |
| Area %: 2 to 15 | Area %: 4 to 20 |

a particle size distribution (at 40 mm) of: D10=13 to 17, D50=35 to 60, D90=650 to 700, %<10 μm=<0.1, 1, 2, 3, 4, 5, 6, 7, 8, 9 or 10%; or
a particle size distribution (at 70 mm) of: D10=13 to 17, D50=24 to 30, D90=540 to 610, %<10 μm=<0.1, 1, 2, 3, 4, 5, 6, 7, 8, 9 or 10%; or
a particle size distribution of: D10=13 to 17, D50=22 to 27, D90=35 to 56, %<9 μm=<0.1-10%; or
% particles of equal to or less than 11.7 μm size of: 0.5 to 5%.

In an embodiment, there is provided an intranasal delivery system, optionally comprising an active nasal delivery device as described herein, comprising: a dry powder formulation comprising a plurality of powder particles of 5-MeO-DMT, or a pharmaceutically acceptable salt thereof, and an active nasal delivery device, optionally as described herein, configured to deliver the particles to the turbinates and olfactory region of the nasal cavity of a subject at a single actuation; wherein the system is operably configured to emit a powder plume comprising 5-MeO-DMT, or a pharmaceutically acceptable salt thereof, having one or more of:
a plume geometry of: angle: 20 to 45 degrees; width: 25 to 55 mm;
a spray pattern of:

| Distance 40 mm: | Distance 70 mm: |
|---|---|
| Dmin(mm): 15 to 25 | Dmin(mm): 25 to 40 |
| Dmax(mm): 35 to 55 | Dmax(mm): 45 to 60 |
| Area (mm²): 600 to 850 | Area (mm²): 1100 to 1700 |
| Area %: 1 to 5 | Area %: 4 to 8 |

a particle size distribution (at 40 mm) of: D10=13 to 17, D50=35 to 60, D90=650 to 700, %<10 μm=<0.1, 1, 2, 3, 4, 5, 6, 7, 8, 9 or 10%; or
a particle size distribution (at 70 mm) of: D10=13 to 17, D50=24 to 30, D90=540 to 610, %<10 μm=<0.1, 1, 2, 3, 4, 5, 6, 7, 8, 9 or 10%; or
% particles of equal to or less than 11.7 μm size of: 0.5 to 5%.

In an embodiment, there is provided an intranasal delivery system, optionally comprising an active nasal delivery device as described herein, comprising: a dry powder formulation comprising a plurality of powder particles of 5-MeO-DMT, or a pharmaceutically acceptable salt thereof, and an active nasal delivery device, optionally as described herein, configured to deliver the particles to the turbinates and olfactory region of the nasal cavity of a subject at a single actuation; wherein the system is operably configured to emit a powder plume comprising 5-MeO-DMT, or a pharmaceutically acceptable salt thereof, having one or more of:
a plume geometry of: angle: 20 to 35 degrees; width: 25 to 45 mm;
a spray pattern of

| Distance 40 mm: | Distance 70 mm: |
|---|---|
| Dmin(mm): 15 to 25 | Dmin(mm): 15 to 40 |
| Dmax(mm): 20 to 35 | Dmax(mm): 35 to 50 |
| Area (mm²): 390 to 460 | Area (mm²): 800-1200 |
| Area %: 5 to 15 | Area %: 7 to 20 |

a particle size distribution of: D10=13 to 17, D50=22 to 27, D90=35 to 56, %<9 μm=<0.1-10%; or
% particles of equal to or less than 11.7 μm size of: 0.5 to 5%.

In an embodiment, there is provided an intranasal delivery system, optionally comprising an active nasal delivery device as described herein, comprising: a dry powder formulation comprising a plurality of powder particles of 5-MeO-DMT, or a pharmaceutically acceptable salt thereof, and an active nasal delivery device, optionally as described herein, configured to deliver the particles to the turbinates and olfactory region of the nasal cavity of a subject at a single actuation; wherein the system is operably configured to emit a powder plume comprising 5-MeO-DMT, or a pharmaceutically acceptable salt thereof, having one or more of:
a plume geometry of: angle: 27 or 40 degrees; width: 33 or 50 mm;
a spray pattern of:

| Distance 40 mm: | Distance 70 mm: |
|---|---|
| Dmin(mm): 21 or 22 | Dmin(mm): 33 or 34 |
| Dmax(mm): 41 or 47 | Dmax(mm): 52 or 54 |
| Area (mm²): 640 or 827 | Area (mm²): 1230 or 1545 |
| Area %: 3 or 4 | Area %: 5 or 7 |

a particle size distribution (at 40 mm) of: D10=15 or 16, D50=38 or 54, D90=684 or 685, %<10 μm=<0.1, 1, 2, 3, 4, 5, 6, 7, 8, 9 or 10%; or
a particle size distribution (at 70 mm) of: D10=15 or 16, D50=27 or 28, D90=558 or 596, %<10 μm=<0.1, 1, 2, 3, 4, 5, 6, 7, 8, 9 or 10%; or
a particle size distribution of: D10=13 to 17, D50=22 to 27, D90=35 to 56, %<9 μm=<0.1-10%; or
% particles of equal to or less than 11.7 μm size of: 0.5 to 5%.

In an embodiment, the powder plume of 5-MeO-DMT, or a pharmaceutically acceptable salt thereof, has a plume geometry of: angle: 22 to 35 degrees; width: 27 to 55 mm; or plume geometry of: angle: 22 to 33 degrees; width: 27 to 42 mm; or a plume geometry of: angle: 25 to 30 degrees; width: 29 to 39 mm; or a plume geometry of: angle: 26 to 28 degrees; width: 32 to 35 mm; or a plume geometry of: angle: 27.5 degrees; width: 34.33 mm; or a plume geometry of: angle: 24.4 degrees; width: 30.30 mm; or a plume geometry of: angle: 24.8 degrees; width: 30.76 mm; or a plume geometry of: angle: 27.4 degrees; width: 34.13 mm; or a plume geometry of angle: 30.5 degrees; width: 38.29 mm; or a plume geometry of angle: 39.2 degrees; width: 50.35 mm; or a plume geometry of angle: 33.43 degrees; width: 52.25 mm; or a plume geometry of: angle: 28 degrees; width: 34 mm; or a plume geometry of angle: 24 degrees; width: 30 mm; or a plume geometry of: angle: 25 degrees; width: 31 mm; or a plume geometry of: angle: 27 degrees; width: 34 mm; a plume geometry of: angle: 31 degrees; width: 38 mm or a plume geometry of angle: 20, 21, 22, 23, 24, 25, 26, 27, 28, 29, 30, 31, 32, 33, 34, 35, 36, 37, 38, 39, 40, 41, 42, 43, 44, 45 or 46 degrees; width: 26, 27, 28, 29, 30, 31, 32, 33, 34, 35, 36, 37, 38, 39, 40, 41, 42, 43, 44, 45, 46, 47, 48, 49, 50, 51, 52, 53, 54, 55 or 56 mm.

In an embodiment, the powder plume of 5-MeO-DMT, or a pharmaceutically acceptable salt thereof, has a spray pattern of:

| Distance 40 mm: | Distance 70 mm: |
|---|---|
| Dmin(mm): 18 to 22 | Dmin(mm): 20 to 30 |
| Dmax(mm): 25 to 30 | Dmax(mm): 40 to 45 |
| Area (mm²): 400 to 450 | Area (mm²): 900-1100 |
| Area %: 7 to 13 | Area %: 9 to 15 |

or

| Distance 40 mm: | Distance 70 mm: |
|---|---|
| Dmin(mm): 19 to 21 | Dmin(mm): 24 to 28 |
| Dmax(mm): 27 to 29 | Dmax(mm): 42 to 44 |
| Area (mm²): 425 to 435 | Area (mm²): 950-1050 |
| Area %: 8 to 11 | Area %: 12 to 14 |

In an embodiment, the powder plume of 5-MeO-DMT, or a pharmaceutically acceptable salt thereof, has a spray pattern of:

| Distance 40 mm: | Distance 70 mm: |
|---|---|
| Dmin(mm): 20.43 or 20.17 or 17.79 or 20.09 | Dmin(mm): 20.84 or 29.23 or 30.67 or 31.10 |
| Dmax(mm): 27.59 or 24.32 or 29.68 or 27.46 | Dmax(mm): 43.69 or 38.83 or 42.82 or 43.31 |
| Area (mm²): 431.0 or 423.7 or 405.6 or 449.0 | Area (mm²): 1048.5 or 919.4 or 1038.8 or 1029.2 |
| Area %: 8.1 or 7.8 or 7.5 or 8.3 | Area %: 10.3 or 10.1 or 9.0 or 10.1 |

or

| Distance 40 mm: | Distance 70 mm: |
|---|---|
| Dmin(mm): 21.45 or 22.10 | Dmin(mm): 33.43 or 33.43 |
| Dmax(mm): 40.66 or 47.23 | Dmax(mm): 52.25 or 53.50 |
| Area (mm²): 639.6 or 827.1 | Area (mm²): 1229.6 or 1544.9 |
| Area %: 2.7 or 3.5 | Area %: 5.3 or 6.6 |

or

| Distance 40 mm: | Distance 70 mm: |
|---|---|
| Dmin(mm): 20 or 18 | Dmin(mm): 21 or 29 or 31 |
| Dmax(mm): 28 or 24 or 27 | Dmax(mm): 44 or 39 or 43 |
| Area (mm²): 431 or 424 | Area (mm²): 1049 or 919 |
| or 406 or 449 | or 1039 or 1029 |
| Area %: 8 | Area %: 9 or 10 |

or

| Distance 40 mm: | Distance 70 mm: |
|---|---|
| Dmin(mm): 18 to 20 | Dmin(mm): 20 to 32 |
| Dmax(mm): 24 to 28 | Dmax(mm): 39 to 45 |
| Area (mm²): 405 to 450 | Area (mm²): 915 to 1050 |
| Area %: 7 to 9 | Area %: 9 to 11 |

In an embodiment, the particle size distribution of the powder plume of 5-MeO-DMT, or a pharmaceutically acceptable salt thereof, is: D10=15.54 or 15.05 or 15.89 or 15.31, D50=26.8 or 28.21 or 38.27 or 53.92, D90=558.4 or 595.9 or 683.8 or 385.3, %<10 μm=5.45 or 3.01 or 3.90 or 3.79; or D10=10.4 or 10.7, D50=21.0 or 22.8, D90=38.4 or 14.9, %<10 μm=8.65% or 8.35%; or D10=13, 14, 15, 16 or 17, D50=22, 23, 24, 25, 26 or 27, D90=35, 36, 37, 38, 39, 40, 41, 42, 43, 44, 45, 46, 47, 48, 49, 50, 51, 52, 53, 54, 55 or 56, %<9 μm=<0.1, 1, 2, 3, 4, 5, 6, 7, 8, 9 or 10%.

In an embodiment, the % particles of equal to or less than 11.7 μm size of the powder plume of 5-MeO-DMT, or a pharmaceutically acceptable salt thereof, is: 0.5 to 5%, 0.6 to 4%, 0.7 to 3%, 0.8 to 2%, 0.9 to 1%. In an embodiment, the active nasal delivery has an actuation force of between 30 and 60N. In an embodiment, the actuation force is between 40 and 50N. In an embodiment, the actuation force is 41, 42, 43, 44, 45, 46, 47, 48 or 49N. In an embodiment, the actuation force is 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80N. In an embodiment, the actuation force is 36N. In an embodiment, the actuation force is 37N. In an embodiment, the actuation force is 38N. In an embodiment, the actuation force is 39N. In an embodiment, the actuation force is 36N.

In an embodiment, the dry powder formulation comprising a plurality of powder particles of 5-MeO-DMT, or a pharmaceutically acceptable salt thereof, comprises a crystalline form of 5-MeO-DMT, or a pharmaceutically acceptable salt thereof, as described herein.

In an embodiment, the dry powder formulation comprising a plurality of powder particles of 5-MeO-DMT, or a pharmaceutically acceptable salt thereof, has a moisture content of <5%, <4%, <3%, <2%, or <1%. In an embodiment, the moisture content is <2%, <1.9%, <1.8%, <1.7%, <1.6%, <1.5%, <1.4%, <1.3%, <1.2% or <1.1%. In an embodiment, the dry powder formulation comprising a plurality of powder particles of 5-MeO-DMT, or a pharmaceutically acceptable salt thereof, has <5%, <4%, <3%, <2%, <1%, <0.9%, <0.8%, <0.7%, <0.6%, <0.5%, <0.4%, <0.3%, <0.2%, <0.1%, <0.09%, <0.08%, <0.07%, <0.06%, <0.05%, <0.04%, <0.03%, <0.02% or <0.01% by weight of a hydroxyl impurity. In an embodiment, the dry powder formulation comprising a plurality of powder particles of 5-MeO-DMT, or a pharmaceutically acceptable salt thereof, has <5%, <4%, <3%, <2%, <1%, <0.9%, <0.8%, <0.7%, <0.6%, <0.5%, <0.4%, <0.3%, <0.2%, <0.1%, <0.09%, <0.08%, <0.07%, <0.06%, <0.05%, <0.04%, <0.03%, <0.02% or <0.010% of by weight of any one impurity.

In an embodiment, the dry powder formulation comprising a plurality of powder particles of 5-MeO-DMT, or a pharmaceutically acceptable salt thereof, has <5%, <4%, <3%, <2%, <1%, <0.9%, <0.8%, <0.7%, <0.6%, <0.5%, <0.4%, <0.3%, <0.2%, <0.1%, <0.09%, <0.08%, <0.07%, <0.06%, <0.05%, <0.04%, <0.03%, <0.02% or <0.01% of by weight of any impurity. In an embodiment, the impurity profile is determined by RP-HPLC. In an embodiment, the % particles of equal to or less than 11.7 μm size of the powder plume is determined by Next Generation Impactor and HPLC. In an embodiment, the moisture content is determined by Karl Fisher coulometric titration.

In an embodiment, the plume geometry is analysed using the Proveris SprayVIEW apparatus (or equivalent) in conjunction with the Proveris automated actuation device. In an embodiment, Analysis is performed at one distance (7.0 cm). In an embodiment, the settings are as follows: Orifice tip distance (cm): 7.0, Frame rate (Hz): 500, Number of images 250, Lens aperture 2.0, Camera position from horizontal (cm): 27.0, Camera height (cm): 8.0, Laser position (cm): 5.2, Laser depth (cm): 5.3, Laser height (cm): 13.2, Actuator position (cm): 7.0, Plume orientation: 0 deg, Palette: Gradient, Arm 1/Arm 2(%): 20-30%, Evacuation time (ms): 1000, Setting time (ms): 1000. In an embodiment, the spray pattern is determined using the Proveris SprayVIEW apparatus (or equivalent) in conjunction with the Proveris automated actuation device. In an embodiment, analysis is performed at two distances (4.0 cm and 7.0 cm). In an embodiment, the settings are as above for the 7.0 cm distance and as follows for the 4.0 cm distance (where different from the settings used for 7.0 cm): Orifice tip distance (cm): 4.0, Camera position from horizontal (cm): 8.0 and Camera height (cm): 22.

In an embodiment, the particle size distribution is determined by laser diffraction using a Malvern Mastersizer (or equivalent). In an embodiment, the settings are as follows: Instrument: Malvern Mastersizer 3000 with Malvern software (or equivalent), Sampling handling Unit: Hydro MV dispersion unit, Material Refractive Index: 1.590, Absorption Refractive Index: 0.001, Dispersant Refractive Index: 1.391 (2,2,4-trimethylpentane), Obscuration Limits: 10-20%, Sonification time: Externally sonicated for 120 secs during sample preparation prior to addition to Hydro MV, Stirrer Speed: 3000 rpm, Measurement time: 30 secs, Background time: 30 secs, Dispersant: 2,2,4-trimethylpentane (RI=1.391) and Lecithin 0.05% w/w, degassed and equilibrated to ambient temperature.

In an embodiment, the aerodynamic particle size distribution (DISP) is determined by a method based on USP <601>, using the Proveris Sprayview and a Copley Next Generation Impactor (NGI) or equivalent, complying with USP/Ph.Eur. In an embodiment, standard solutions are prepared based on the label claim for the drug product (xmg per 100 ml diluent) where x=label claim. In an embodiment, the settings are as follows: Actuation acceleration: 5000 mm/s/s, Actuation velocity: 70 mm/s, Symmetric: Yes, Initial delay: 0 ms, Hold time: 100 ms, Final delay: 0 ms, Stroke length: 14 mm, One shot is fired into the NGI. Weigh the device prior to (W1) and after firing (W2) to calculate shot weight (W3). W1-W2=shot weight (W3), Add 5 ml of test solvent to each NGI cup then place on the NGI gentle rocker for 5 minutes. Quantitatively wash the expansion chamber, bungs, inlet cone, all cups and the Proveris collar with diluent into the correct flask size and make to volume. Assay is determined via HPLC.

In an embodiment, the particle size distribution (PSD) is determined by laser diffraction. In an embodiment, the analysis is performed using Sympatec instrumentation with R5 lens and a dispersal pressure of 3 bar. The intranasal delivery system/device is held in a clamp stand and positioned central with the extractor and so the tip of the device is 3 cm from the mid-point of the laser. After referencing, the device is manually/hand actuated so the powder passes through the laser beam, which takes a reading. Readings are performed with an R5 lens, in triplicate and then an average calculated.

### Example 6: Items

Item 1: An intranasal pharmaceutical composition comprising a salt of 5-methoxy-N,N-dimethyltryptamine (5-MeO-DMT salt) for use, as a medicament, wherein the pharmaceutical composition comprises: a first dose of the 5-MeO-DMT salt, for administration to a first nostril of a subject; and a second dose of the 5-MeO-DMT salt, for second administration to a second nostril of the subject. Item 2: The intranasal pharmaceutical composition of item 1, wherein the 5-MeO-DMT salt is amorphous, crystalline or is substantially polymorphically pure. Item 3: The intranasal pharmaceutical composition of item 1 or item 2, wherein the pharmaceutical composition comprises one or more pharmaceutically acceptable carriers or excipients. Item 4: The intranasal pharmaceutical composition of any one of items 1 to 3, wherein the pharmaceutical composition is a dry powder. Item 5: The intranasal pharmaceutical composition of any one of items 1 to 4, wherein the 5-MeO-DMT salt is non-hygroscopic. Item 6: The intranasal pharmaceutical composition of any one of items 1 to 5, wherein the pharmaceutical composition comprises a mucoadhesive, optionally the mucoadhesive is hydroxypropyl methyl cellulose (HPMC). Item 7: The intranasal pharmaceutical composition of any one of items 1 to 6, wherein the mass ratio of the first dose of 5-MeO-DMT salt to the second dose of 5-MeO-DMT salt is from 1:11 to 11:1, optionally from 2:10 to 10:2, further optionally from 4:8 to 8:4, and still further optionally the mass ratio is the same. Item 8: The intranasal pharmaceutical composition of any one of items 1 to 7, wherein the first dose of the 5-MeO-DMT salt is lower, equal, or higher than the second dose of the 5-MeO-DMT salt. Item 9: The intranasal pharmaceutical composition of any one of items 1 to 8, wherein the first dose of the 5-MeO-DMT salt is lower than the second dose of the 5-MeO-DMT salt. Item 10: The intranasal pharmaceutical composition of any one of items 1 to 9, wherein the anion of the first dose of the 5-MeO-DMT salt is a benzoate, hydrobromide, hydrochloride, phosphate, fumarate, oxalate, tartrate, benzenesulfonate, tosylate, glycolate, ketoglutarate, malate, saccharinate or succinate salt. Item 11: The intranasal pharmaceutical composition of any one of items 1 to 10, wherein the anion of the second dose of the 5-MeO-DMT salt is a benzoate, hydrobromide, hydrochloride, phosphate, fumarate, oxalate, tartrate, benzenesulfonate, tosylate, glycolate, ketoglutarate, malate, saccharinate or succinate salt. Item 12: The intranasal pharmaceutical composition of any one of items 1 to 11, wherein the first dose of the 5-MeO-DMT salt and the second dose of the 5-MeO-DMT salt are the same salt. Item 13: The intranasal pharmaceutical composition of any one of items 1 to 11, wherein the first dose of the 5-MeO-DMT salt and the second dose of the 5-MeO-DMT salt are not the same salt. Item 14: The intranasal pharmaceutical composition of any one of items 1 to 11, wherein the 5-MeO-DMT salt is 5-MeO-DMT benzoate. Item 15: The intranasal pharmaceutical composition of item 14, wherein the 5-MeO-DMT benzoate is crystalline. Item 16: The intranasal pharmaceutical composition of item 15, wherein the crystalline 5-MeO-DMT benzoate is characterised by one or more peaks in an X-ray powder diffraction (XRPD) diffractogram at a 2θ value of 17.5°±0.1°, 17.7°±0.1° and 21.0°±0.1° using an X-ray wavelength of 1.5406 Å. Item 17: The intranasal pharmaceutical composition of any one of items 14 to 16, wherein the first and second dose of the 5-MeO-DMT salt benzoate forms a combined dose of 30, 20, 18, 16, 15, 14, 13, 12, 11, 10, 9, 8, 7, 6, 5, 4, 3, 2 or 1 mg of the 5-MeO-DMT benzoate. Item 18: The intranasal pharmaceutical composition of item 17, wherein the first and second dose of the 5-MeO-DMT salt benzoate forms a combined dose of 12 mg of the 5-MeO-DMT benzoate. Item 19: The intranasal pharmaceutical composition of item 18, wherein the first dose of 5-MeO-DMT benzoate is 4 mg and the second dose of 5-MeO-DMT benzoate is 8 mg. Item 20: The intranasal pharmaceutical composition of any one of items 14 to 19, wherein the pharmaceutical composition comprises 5-MeO-DMT hydrobromide in place of the 5-MeO-DMT benzoate, and wherein the pharmaceutical composition comprises substantially the same dosage amount of the active 5-MeO-DMT cation. Item 21: The intranasal pharmaceutical composition of item 20, wherein the 5-MeO-DMT hydrobromide is crystalline. Item 22: The intranasal pharmaceutical composition item 21, wherein the crystalline 5-MeO-DMT hydrobromide is characterised by one or more of: peaks in an x-ray powder diffraction (XRPD) diffractogram at 2θ values of 14.6°±0.1°, 16.8°±0.1°, 20.8°±0.1°, 24.3°±0.1°, 24.9°±0.1°, and 27.5°±0.1° as measured using an x-ray wavelength of 1.5406 Å; peaks in an x-ray powder diffraction (XRPD) diffractogram at 2θ values of 14.6°±0.1°, 21.6°±0.1°, and 24.3°±0.1° as measured using an x-ray wavelength of 1.5406 Å; peaks in an x-ray powder diffraction (XRPD) diffractogram at 2θ values of 18.6°±0.1°, 19.7°±0.1°, and 24.8°±0.1° as measured using an x-ray wavelength of 1.5406; or peaks in an x-ray powder diffraction (XRPD) diffractogram at 2θ values of 14.6°±0.1°, 20.8°±0.1°, 21.6°±0.1°, 24.3°±0.1°, and 25.4°±0.1° as measured using an x-ray wavelength of 1.5406 Å. Item 23: The intranasal pharmaceutical composition of any one of items 1 to 22, wherein the second dose of the 5-MeO-DMT salt is for administration within 30, 20, 15, 10, 5 or 2 minutes after the first dose of the 5-MeO-DMT salt. Item 24: The intranasal pharmaceutical composition of any one of items 1 to 23, wherein the second dose of the 5-MeO-DMT salt is for administration while the subject being treated is experiencing one or more mystical effects from the first dose of the 5-MeO-DMT salt. Item 25: A pharmaceutical kit comprising a first single use intranasal applicator comprising a dry powder intranasal pharmaceutical composition of 5-MeO-DMT benzoate and a second single use intranasal applicator comprising a dry powder intranasal pharmaceutical composition of 5-MeO-DMT benzoate, wherein the dose of 5-MeO-DMT benzoate in the second intranasal applicator is higher than that in the first, optionally, the dose of 5-MeO-DMT benzoate in the first applicator is 4 mg and the dose in the second applicator is 8 mg

1.-30. (canceled)

31. A kit, comprising:
a first pharmaceutical composition comprising (i) 5-methoxy-N,N-dimethyltryptamine (5-MeO-DMT) free base, or a pharmaceutically acceptable salt, prodrug, hydrate, ester, co-crystal or deuterated form thereof; and (ii) one or more pharmaceutically acceptable carriers or excipients;
a second pharmaceutical composition comprising (i) 5-methoxy-N,N-dimethyltryptamine (5-MeO-DMT) free base, or a pharmaceutically acceptable salt, prodrug, hydrate, ester, co-crystal or deuterated form thereof; and (ii) one or more pharmaceutically acceptable carriers or excipients; and
instructions for use of the kit;
wherein, the second pharmaceutical composition comprises a higher dose of 5-MeO-DMT free base, or an equivalent dose of the pharmaceutically acceptable salt, prodrug, hydrate, ester, co-crystal or deuterated form thereof, than the first pharmaceutical composition.

32. The kit of claim 31, wherein the first and second pharmaceutical compositions are for intranasal administration.

33. The kit of claim 32, wherein the instructions provide that the first pharmaceutical composition is administered to one nostril and the second pharmaceutical composition is administered to the other nostril.

34. The kit of claim 33, wherein the instructions provide that the second pharmaceutical composition is administered within 30 minutes after the first pharmaceutical composition
wherein the instructions provide that the second pharmaceutical composition is administered within 20 minutes after the first pharmaceutical composition;
wherein the instructions provide that the second pharmaceutical composition is administered within 15 minutes after the first pharmaceutical composition;
wherein the instructions provide that the second pharmaceutical composition is administered within 10 minutes after the first pharmaceutical composition; or
wherein the instructions provide that the second pharmaceutical composition is administered within 5 minutes after the first pharmaceutical composition.

35.-38. (canceled)

39. The kit of claim 31, wherein the first pharmaceutical composition comprises 1, 2, 3, 4, 5 or 6 mg 5-MeO-DMT free base, or an equivalent dose of the pharmaceutically acceptable salt, prodrug, hydrate, ester, co-crystal or deuterated form thereof; and/or
wherein the second pharmaceutical composition comprises 4, 5, 6, 7, 8, 9, 10, 11 or 12 mg 5-MeO-DMT free base, or an equivalent dose of the pharmaceutically acceptable salt, prodrug, hydrate, ester, co-crystal or deuterated form thereof.

40. (canceled)

41. The kit of claim 31, wherein the first pharmaceutical composition comprises 4 mg 5-MeO-DMT free base, or an equivalent dose of the pharmaceutically acceptable salt, prodrug, hydrate, ester, co-crystal or deuterated form thereof; and/or
wherein the second pharmaceutical composition comprises 8 mg 5-MeO-DMT free base, or an equivalent dose of the pharmaceutically acceptable salt, prodrug, hydrate, ester, co-crystal or deuterated form thereof.

42.-43. (canceled)

44. The kit of claim 31, wherein the first and second pharmaceutical compositions are dry powders.

45. The kit of claim 31, wherein the first and second pharmaceutical compositions comprise a mucoadhesive, and/or hydroxypropyl methyl cellulose (HPMC).

46. The kit of claim 31, wherein the first and second pharmaceutical compositions comprise a 5-MeO-DMT salt selected from a benzoate, hydrobromide, hydrochloride, phosphate, fumarate, oxalate, tartrate, benzenesulfonate, tosylate, glycolate, ketoglutarate, malate, saccharinate or succinate salt.

47. The kit of claim 46, wherein the 5-MeO-DMT salt is amorphous;
wherein the 5-MeO-DMT salt is crystalline;
wherein the 5-MeO-DMT salt is crystalline 5-MeO-DMT benzoate; or
wherein the 5-MeO-DMT salt is crystalline 5-MeO-DMT benzoate as characterized by one or more peaks in an X-ray powder diffraction (XRPD) diffractogram at a 2θ value of 17.5°±0.1°, 17.7°±0.1° and 21.0°±0.1° using an X-ray wavelength of 1.5406 Å.

48.-50. (canceled)

51. The kit of claim 31 wherein administration of the first and second pharmaceutical compositions provides a complete mystical experience to treat one or more conditions or disorders in a patient in need thereof.

52. The kit of claim 51, wherein the one or more conditions or disorders are selected from treatment resistant depression, major depressive disorder, depression, persistent depressive disorder, alcohol use disorder, anxiety disorder, post-traumatic stress disorder (PTSD), body dysmorphic disorder, obsessive-compulsive disorder, eating disorder, psychoactive substance abuse and substance abuse.

53.-57. (canceled)

58. The kit of claim 31, wherein:
the first and second pharmaceutical compositions are for intranasal administration;
the instructions provide that the first pharmaceutical composition is administered to one nostril and the second pharmaceutical composition is administered to the other nostril;
the instructions provide that the second pharmaceutical composition is administered within 15 minutes after the first pharmaceutical composition;
the first pharmaceutical composition comprises 4 mg 5-MeO-DMT free base, or an equivalent dose of a benzoate or hydrobromide salt thereof;
the second pharmaceutical composition comprises 8 mg 5-MeO-DMT free base, or an equivalent dose of a benzoate or hydrobromide salt thereof;
the first and second pharmaceutical compositions are dry powders; and
administration of the first and second pharmaceutical compositions provides a complete mystical experience in a patient having depression.

59.-60. (canceled)

61. The kit of claim 31, comprising: a first intranasal delivery device comprising the first pharmaceutical composition; and a second intranasal delivery device comprising the second pharmaceutical composition.

62.-89. (canceled)

90. A method for treating one or more conditions or disorders in a patient in need thereof, comprising administering a first dose of 5-MeO-DMT free base, or a pharmaceutically acceptable salt, prodrug, hydrate, ester, co-crystal or deuterated form thereof, or a pharmaceutical composition thereof, followed by administering a second dose of 5-MeO-DMT free base, or a pharmaceutically acceptable salt, prodrug, hydrate, ester, co-crystal or deuterated form thereof, or a pharmaceutical composition thereof,
wherein the second dose is higher than the first dose.

91. The method of claim 90, wherein the method provides a complete mystical experience.

92. The method of claim 90, wherein the route of administration is intranasal.

93. The method of claim 90, wherein the first dose is administered to one nostril and the second dose is administered to the other nostril.

94. The method of claim 90, wherein
the second dose is administered within 30 minutes after the first dose;
the second dose is administered within 20 minutes after the first dose;
the second dose is administered within 15 minutes after the first dose;
the second dose is administered within 10 minutes after the first dose; or
the second dose is administered within 5 minutes after the first dose.

95.-98. (canceled)

99. The method of claim 90, wherein 1, 2, 3, 4, 5 or 6 mg 5-MeO-DMT free base or an equivalent dose of the pharmaceutically acceptable salt, prodrug, hydrate, ester, co-crystal or deuterated form thereof, or a pharmaceutical composition thereof is administered as the first dose; and/or
4, 5, 6, 7, 8, 9, 10, 11 or 12 mg 5-MeO-DMT free base, or an equivalent dose of the pharmaceutically acceptable salt, prodrug, hydrate, ester, co-crystal or deuterated form thereof, or a pharmaceutical composition thereof is administered as the second dose.

100. (canceled)

101. The method of claim 90, wherein 4 mg 5-MeO-DMT free base or an equivalent dose of the pharmaceutically acceptable salt, prodrug, hydrate, ester, co-crystal or deuterated form thereof, or a pharmaceutical composition thereof is administered as the first dose; and/or
wherein 8 mg 5-MeO-DMT free base or an equivalent dose of the pharmaceutically acceptable salt, prodrug, hydrate, ester, co-crystal or deuterated form thereof, or a pharmaceutical composition thereof is administered as the second dose.

102.-103. (canceled)

104. The method of claim 90, wherein the pharmaceutical composition is a dry powder.

105. The method of claim 90, wherein the pharmaceutical composition comprises a mucoadhesive, and/or hydroxypropyl methyl cellulose (HPMC).

106. The method of claim 90, wherein the 5-MeO-D MT salt is selected from a benzoate, hydrobromide, hydrochloride, phosphate, fumarate, oxalate, tartrate, benzenesulfonate, tosylate, glycolate, ketoglutarate, malate, saccharinate or succinate salt.

107. The method of claim 90, wherein the 5-MeO-DMT salt is amorphous;
wherein the 5-MeO-DMT salt is crystalline;
wherein the 5-MeO-DMT salt is crystalline 5-MeO-DMT benzoate; or
wherein the 5-MeO-DMT salt is crystalline 5-MeO-DMT benzoate as characterized by one or more peaks in an X-ray powder diffraction (XRPD) diffractogram at a 2θ value of 17.5°±0.1°, 17.7°±0.1° and 21.0°±0.1° using an X-ray wavelength of 1.5406 Å.

108.-110. (canceled)

111. The method of claim 90, wherein the one or more conditions or disorders are selected from: treatment resistant depression, major depressive disorder, depression, persistent depressive disorder, alcohol use disorder, anxiety disorder, post-traumatic stress disorder (PTSD), body dysmorphic disorder, obsessive-compulsive disorder, eating disorder, psychoactive substance abuse and/er substance abuse.

112.-115. (canceled)

116. The method of claim 90, wherein:
the route of administration is intranasal;
the first dose is administered to one nostril and the second dose is administered to the other nostril;
the second dose is administered within 15 minutes after the first dose;
4 mg 5-MeO-DMT free base, or an equivalent dose of a benzoate or hydrobromide salt thereof, or a pharmaceutical composition thereof, is administered as the first dose;
8 mg of 5-MeO-DMT free base or an equivalent dose of a benzoate or hydrobromide salt thereof, or a pharmaceutical composition thereof, is administered as the second dose;
administration of 5-MeO-DMT free base, or an equivalent dose of a benzoate or hydrobromide salt thereof, provide a complete mystical experience in the patient; and
the one or more conditions or disorders is depression.

117. (canceled)

118. The method of claim 90, wherein administration of 5-MeO-DMT free base or the pharmaceutically acceptable salt, prodrug, hydrate, ester, co-crystal or deuterated form thereof, or the pharmaceutical composition thereof, to the patient is performed by the patient or a medical professional.

119. (canceled)

120. The method of claim 90, wherein the one or more conditions or disorders have been clinically diagnosed in accordance with accepted medical practice.

121. The method of claim 90, wherein administration of 5-MeO-DMT free base or the pharmaceutically acceptable salt, prodrug, hydrate, ester, co-crystal or deuterated form thereof, or the pharmaceutical composition thereof, provides a complete mystical experience,
wherein the complete mystical experience is identified through achievement of at least 60% of the maximum possible score in each of the four subscales comprising mystical, positive mood, transcendence of time and space, and ineffability of the 30-claim revised Mystical Experience Questionnaire (MEQ30), and/or through achievement of at least 60% of the maximum possible score of the Oceanic Boundlessness (OBN) dimension of the Altered States of Consciousness (ASC) questionnaire; and/or
wherein the complete mystical experience is induced within 5, 10, 15, 20, 25, or 30 minutes of administration; and/or
wherein the complete mystical experience is resolved within 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 minutes of administration.
